(19) United States
(12) Patent Application Publication
(10) Pub. No.: US 2026/0232736 A1
(43) Pub. Date: Aug. 13, 2026

(54) CELL THERAPY FOR DIABETES IN TRANSPLANT RECIPIENTS

(71) Applicant: Vertex Pharmaceuticals Incorporated, Boston, MA (US)

(72) Inventors: Felicia J. Pagliuca, Boston, MA (US); Bote Bruinsma, Boston, MA (US); Jason Gaglia, Boston, MA (US); Jack Varon, Boston, MA (US)

(73) Assignee: Vertex Pharmaceuticals Incorporated, Boston, MA (US)

(21) Appl. No.: 19/412,239

(22) Filed: Dec. 8, 2025

Related U.S. Application Data
(60) Provisional application No. 63/733,042, filed on Dec. 12, 2024.
(60) Provisional application No. 63/832,921, filed on Jun. 30, 2025.

(51) Int. Cl. A61K 35/39; A61K 31/365; A61K 31/436; A61K 31/5377; A61K 39/00; A61K 39/395; A61P 3/10; A61P 37/06

(52) U.S. Cl. CPC A61K 35/39; A61K 31/365; A61K 31/436; A61K 31/5377; A61K 39/3955; A61P 3/10; A61P 37/06; A61K 2039/505

(57) ABSTRACT

Disclosed herein are methods of treating a subject having diabetes with a population of stem cell-derived islet cells and one or more immunosuppressive reagents, such as an anti-thymocyte globulin binding moiety. The subject may have received an organ transplant.

### CROSS REFERENCE TO RELATED APPLICATIONS

This claims the benefit of U.S. Provisional Application No. 63/832,921, filed Jun. 30, 2025, and U.S. Provisional Application No. 63/733,042, filed Dec. 12, 2024, which are both incorporated by reference herein in their entirety.

### BACKGROUND

Generation of stem cell derived B-cells can provide a potentially useful step toward the generation of islets and pancreatic organs. One of the rapidly growing diseases that may be treatable by stem cell derived tissues is diabetes. Type 1 diabetes results from autoimmune destruction of β-cells in the pancreatic islet. Type 2 diabetes results from peripheral tissue insulin resistance and β-cell dysfunction. Diabetic patients, particularly those suffering from type 1 diabetes, can potentially be cured through transplantation of new β-cells. Patients transplanted with cadaveric human islets can be made insulin-independent for 5 years or longer via this strategy, but this approach is limited because of the scarcity and quality of donor islets. Generation of an unlimited supply of human B-cells from stem cells can extend this therapy to millions of new patients and can be an important test case for translating stem cell biology into the clinic. For certain stem cell-derived islet therapies, there may also be a need or desire for immunosuppressive or supplemental treatment.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### SUMMARY

Methods of treating a subject having diabetes that include treating the subject with a composition including a population of stem cell-derived islet cells and one or more immunosuppressive agents are described herein. In some examples, the subject previously received an organ transplant. Compositions, devices, and systems for performing these methods are also disclosed.

In some embodiments, the disclosure provides for a method of treating a subject having diabetes, comprising administering to the subject: a) a composition comprising a population of stem cell-derived islet cells, and b) one or more immunosuppressive agents; wherein the subject previously received an organ transplant. In some embodiments, the subject previously received a kidney transplant. In some embodiments, the subject previously received a liver transplant. In some embodiments, the composition comprises about 1×10⁸ to about 10×10⁸ cells. In some embodiments, the composition comprises a plurality of C-peptide-positive, ISL1-positive cells. In some embodiments, the composition comprises a plurality of NKX6.1-positive, ISL1-positive cells. In some embodiments, (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, or 80-90% of the cells in the composition express C-peptide and ISL1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the composition express glucagon but not somatostatin; and/or (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the composition express somatostatin but not glucagon. In some embodiments, a) 35-65% of the cells in the composition express C-peptide and ISL1; b) 4-25%, of the cells in the composition express glucagon but not somatostatin; and c) 1-10% of the cells in the composition express somatostatin but not glucagon. In some embodiments, less than 40%, less than 35%, less than 30%, less than 25%, less than 20%, less than 18%, less than 15%, less than 12%, or less than 10% of the cells in the composition express VMAT1 but not C-peptide. In some embodiments, no less than 50%, 40%, 30%, or 20% of the cells in the composition are NKX6.1⁺/ISL1⁺ cells. In some embodiments, no less than 20% of the cells in the composition are NKX6.1⁺/ISL1⁺ cells. In some embodiments, no less than 40%, 35%, 30%, 26%, 25%, 20%, 15% or 10% of the cells in the composition are NKX6.1⁻/ISL1⁺ cells. In some embodiments, between 5-25%, 5-40%, 5-35%, or 8-20% of the cells in the composition are NKX6.1⁻/ISL1⁺ cells. In some embodiments, no more than 50%, 45%, 40%, 35%, 30%, or 25% of the cells in the composition are NKX6.1⁺/ISL1⁻ cells. In some embodiments, between 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, 40-50% of the cells in the composition are NKX6.1⁺/ISL1⁺ cells. In some embodiments, between 8-20%, 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, or 40-50% of the cells in the composition are NKX6.1⁻/ISL1⁺ cells. In some embodiments, between 3-50%, 3-40%, 3-30%, 3-25%, 3-20%, 3-15%, 3-10%, or 3-5% of the cells in the composition are NKX6.1⁺/ISL1⁻ cells. In some embodiments, less than 6% or between 0-6%, 0-4%, 0-2%, 0-1%, 1-6%, 1-5%, 1-4%, 1-3%, 1-2%, 2-3%, or 3-6% of the cells in the composition are NKX6.1⁻/ISL1⁻ cells. In some embodiments, wherein: a. at least 30% of the cells in the composition are NKX6.1-positive, ISL1-positive cells; b. at least 15% of the cells in the composition are NKX6.1-negative, ISL1-positive cells; c. less than 12% of the cells in the composition are NKX6.1-negative, ISL1-negative cells; and/or d. less than 20% of the cells in the composition are NKX6.1-positive, ISL1-negative cells. In some embodiments, the composition comprises NKX6.1⁺/ISL1⁺ cells that display a glucose stimulated insulin secretion (GSIS) response in vitro. In some embodiments, the composition comprises NKX6.1⁺/ISL1⁺ cells that display a glucose stimulated insulin secretion (GSIS) response in vivo. In some embodiments, a plurality of cells in the composition are in one or more cell clusters. In some embodiments, the one or more cell clusters are about 50 μm to about 500 μm, about 50 μm to about 400 μm, about 50 μm to about 300 μm, about 60 μm to about 400 μm, about 60 μm to about 300 μm, about 60 μm to about 250 μm, about 75 μm to about 400 μm, about 75 μm to about 300 μm, about 75 μm to about 250 μm, about 125 μm to about 225 μm, about 130 μm to about 160 μm, about 170 μm to about 225 μm, about 140 μm to about 200 μm, about 140 μm to about 170 μm, about 160 μm to about 220 μm, about 170 μm to about 215 μm, or about 170 μm to about 200 μm in diameter. In some embodiments, the composition does not comprise any clusters greater than 200, 250, 300, 400, 450, or 500 μm in diameter. In some embodiments, the one or more immunosuppressive agents are an anti-thymocyte globulin binding moieties (e.g., ATG-R, ATG-F, ATGAM), an anti-CD3 binding moiety (e.g., OKT3), an anti-CD25 binding moiety (e.g., daclizumab or basiliximab), an anti-CD52 binding moiety (e.g., alemtuzumab), or a high dose of an immunosuppression maintenance agent (e.g., bolus glucocorticosteroids or intravenous cyclosporine [CsA]). In some embodiments, the one or more immunosuppressive agents is an anti-thymocyte globulin binding moiety. In some embodiments, the anti-thymocyte globulin binding moiety is administered at a dose of less than 6.0 mg/kg (e.g., 1-5.5 mg/kg, 1-4 mg/kg, 2-5.5 mg/kg, 2-4 mg/kg, 1-2 mg/kg, 2.5-3.5 mg/kg, 2.8-3.2 mg/kg, 2.9-3.1 mg/kg or around 3.0 mg/kg). In some embodiments, if the subject was previously administered an anti-thymocyte globulin binding moiety, then the one or more immunosuppressive agents is not an anti-thymocyte globulin binding moiety. In some embodiments, the one or more immunosuppressive agents is an anti-CD25 antibody. In some embodiments, the one or more immunosuppressive agents is basiliximab. In some embodiments, the one or more immunosuppressive agents is an anti-CD52 antibody. In some embodiments, the one or more immunosuppressive agents is alemtuzumab. In some embodiments, the basiliximab is administered at a dose of 10-30 mg, 15-25 mg, 18-22 mg, or 19-21 mg. In some embodiments, the one or more immunosuppressive agents is administered on the same day as the composition comprising a population of stem cell-derived islet cells. In some embodiments, the method further comprises the step of administering the one or more immunosuppressive agents to the subject 2-10 days, 2-8 days, 2-7 days, 2-5 days, 3-8 days, 3-6 days, 4-6 days, 4 days, or 5 days after the composition comprising a population of stem cell-derived islet cells. In some embodiments, the subject was not previously administered a composition comprising a population of stem cell-derived islet cells. In some embodiments, the method comprises administering multiple doses of the one or more immunosuppressive agents. In some embodiments, the method comprises administering multiple doses of the one or more immunosuppressive agents in a single day. In some embodiments, the method comprises administering multiple doses of an immunosuppression-induction agent over several days. In some embodiments, a dose of the one or more immunosuppressive agents is administered at 0.05-3 mg/kg. In some embodiments, the method comprises administering to the subject a Tumor Necrosis Factor-alpha (TNFα) inhibitor. In some embodiments, the subject is administered 10-100 mg of a Tumor Necrosis Factor-alpha (TNFα) inhibitor. In some embodiments, the method comprises administering to the subject sirolimus (e.g., 0.01-5000 mg/kg of sirolimus). In some embodiments, the method comprises administering tacrolimus (e.g., 0.001-0.05 mg/kg of tacrolimus) to the subject. In some embodiments, the method comprises administering to the subject mycophenolate mofetil or mycophenolate sodium. In some embodiments, the method comprises administering to the subject 100-5000 mg of mycophenolate mofetil or mycophenolate sodium. In some embodiments, the subject is administered an antimicrobial agent. In some embodiments, the method further comprises the steps of: i) administering to the subject a second composition comprising a second population of cells, and ii) administering to the subject an anti-CD25 binding moiety (e.g., basiliximab) concurrently with or after the administration of the second composition; wherein the second composition comprises a plurality of cells that express ISL1. In some embodiments, the method further comprises the steps of: i) administering to the subject a second composition comprising a second population of cells, and ii) administering to the subject a vasoactive agent (e.g., pentoxifylline) concurrently with or after the administration of the second composition; wherein the second composition comprises a population of cells that express ISL1. In some embodiments, the subject is administered a first dose of 0.05-3 mg/kg anti-thymocyte globulin binding moiety within 72-24 hours prior to being administered the composition. In some embodiments, the anti-thymocyte globulin moiety is administered via infusion. In some embodiments, the infusion is via a high-flow vein. In some embodiments, the infusion lasts for 2-20, 3-18, 4-15, 5-13, 6-12, 7-11, 8-10, or 9-11 hours. In some embodiments, the subject is not administered any anti-thymocyte globulin binding moiety for a resting period between doses. In some embodiments, the resting period is 1-20, 6-15, 12-24, 10-20, 1-12, 2-11, 3-10, 4-9, 5-8, 5-7, 5-15, 5-20, 8-10, or 9-14 hours. In some embodiments, the first dose of the anti-thymocyte globulin binding moiety administration protocol is infused over a period of 6-12 hours. In some embodiments, any subsequent dose of the anti-thymocyte globulin binding moiety administration protocol, if any, is infused over a period of 6-12 hours. In some embodiments, there are at least 6 hours between the end of one anti-thymocyte globulin binding moiety infusion and the start of a subsequent anti-thymocyte globulin binding moiety infusion. In some embodiments, the total dose of anti-thymocyte globulin binding moiety at the end of the anti-thymocyte globulin binding moiety administration protocol is between: a, 1 mg/kg to 10 mg/kg; b. 2 mg/kg to 9 mg/kg; c. 3 mg/kg to 8 mg/kg; d. 4 mg/kg to 7 mg/kg; or 5 mg/kg to 6 mg/kg. In some embodiments, the total dose of the anti-thymocyte globulin binding moiety administration is 6 mg/kg. In some embodiments, the anti-thymocyte globulin binding moiety administration protocol includes dosing the first dose of anti-thymocyte globulin binding moiety administration at a lower dose than any subsequent dose of anti-thymocyte globulin binding moiety. In some embodiments, the anti-thymocyte globulin binding moiety administration protocol includes monitoring of total white blood cell (WBC) and platelet counts of the subject following a dose of the anti-thymocyte globulin binding moiety; and reducing a subsequent dose of anti-thymocyte globulin binding moiety (e.g., by at least 10%, 20%, 30%, 40%, 50%, 60%, 70%, 80%, 90% or 100%) if: a. the WBC count is between 2,000 and 3,000 cells/mm³; or b. the platelet count is between 50,000 and 75,000 cells/mm³. In some embodiments, the anti-thymocyte globulin binding moiety administration protocol includes monitoring of total white blood cell (WBC) and platelet counts of the subject following a dose of the anti-thymocyte globulin binding moiety; and stopping the anti-thymocyte globulin binding moiety administration if: a. the WBC count falls below 2,000 cells/mm³; or b. the platelet count falls below 50,000 cells/mm³. In some embodiments, the subject is administered 2, 3, 4, 5, 6, or 7 doses of the TNFα inhibitor in the month following the administration of the composition comprising the population of cells. In some embodiments, the subject is administered the TNFα inhibitor about 3 days, about 4 days, about 5 days, about 6 days, about 7 days, about 8 days, about 9 days, about 10 days, about 11 days, about 12 days, about 13 days, about 14 days, and/or about 15 days following the administration of the composition. In some embodiments, the subject is administered 5-500, 5-100, 5-75, 5-50, 10-100, 10-75, 10-50, 30-100, or 30-75 mg TNFα inhibitor for each dose of the TNFα inhibitor. In some embodiments, the TNFα inhibitor is administered subcutaneously and/or intravenously. In some embodiments, the TNFα inhibitor is selected from the group consisting of etanercept, adalimumab, infliximab, certolizumab pegol, and golimumab. In some embodiments, the subject is administered etanercept. In some embodiments, etanercept is administered on the same day as the administration of the composition comprising the population of cells. In some embodiments, etanercept is administered on the same day as the administration of the composition comprising the population of cells (Day 1) followed by further administration on Day 2, Day 3, Day 4, Day 5, Day 6, Day 7, Day 8, Day 9, Day 10, Day 11, Day 12, Day 13, Day 14, and/or Day 15. In some embodiments, etanercept is administered on multiple days starting on the same day as the administration of the composition. In some embodiments, a first administration of etanercept occurs on the same day as the administration of the composition (Day 1) followed by: a. a second administration between Day 2 to Day 6; b. a third administration between Day 5 to Day 10; and/or c. a fourth administration between Day 9 to Day 14; wherein etanercept is not administered on consecutive days. In some embodiments, a first administration of etanercept occurs on the same day as the administration of the composition (Day 1) followed by: a. a second administration between Day 3 to Day 5; b. a third administration between Day 7 to Day 9; and/or c. a fourth administration between Day 10 to Day 12. In some embodiments, a first administration of etanercept occurs on the same day as the administration of the composition (Day 1) followed by: a. a second administration on Day 4; b. a third administration on Day 8; and/or c. a fourth administration on Day 11. In some embodiments, etanercept is administered at a dose of: a. 10 mg to 100 mg; b. 15 mg to 95 mg; c. 20 mg to 90 mg; d. 25 mg to 85 mg; e. 30 mg to 80 mg; f. 35 mg to 75 mg; g. 40 mg to 70 mg; h. 45 mg to 65 mg; and/or i. 50 mg to 60 mg. In some embodiments, etanercept is administered at a dose of 50 mg on the same day as the administration of the composition (Day 1). In some embodiments, etanercept is administered at a dose of 25 mg for any administration occurring on Day 2 or after. In some embodiments, the etanercept administration is intravenous (IV) and/or subcutaneous. In some embodiments, the first etanercept administration is a different administration method than subsequent etanercept administrations. In some embodiments, the first etanercept administration is IV and the subsequent etanercept administrations are subcutaneous. In some embodiments, prior to the administration of the composition comprising the population of cells, the subject received infusion of insulin at a level of at least about 20 U/day, 22 U/day, 24 U/day, 26 U/day, 28 U/day, 30 U/day, 32 U/day, or 34 U/day. In some embodiments, the subject is administered 0.01-1, 0.02-0.3, 0.02-0.15, 0.02-0.12, 0.02-0.08, 0.03-0.07, 0.04-0.15, or 0.05-2 mg/kg sirolimus. In some embodiments, the sirolimus is administered daily. In some embodiments, the sirolimus is administered orally. In some embodiments, the subject is administered 0.001-0.05, 0.003-0.03, 0.004-0.02, 0.006-0.02, 0.008-0.02, 0.01-0.02, 0.009-0.03, or 0.009-0.02 mg/kg tacrolimus. In some embodiments, the tacrolimus is administered daily. In some embodiments, the tacrolimus is administered orally. In some embodiments, the first dose of Sirolimus is 0.05 to 0.2 mg/kg and subsequent doses are 0.05 to 0.1 mg/kg once each morning (qam). In some embodiments, the sirolimus dose is adjusted to maintain a whole blood 24-hour trough target of 10 to 15 ng/mL for the first 3 months after administration of the composition. In some embodiments, the sirolimus dose is adjusted to maintain a whole blood 24-hour trough target of 8 to 12 ng/ml after the first 3 months after administration of the composition. In some embodiments, the administration of tacrolimus is started after the day of the administration of the composition comprising the population of cells (Day 1). In some embodiments, the administration of tacrolimus is started between Day 2 and Day 5 of the administration of the composition. In some embodiments, the administration of tacrolimus is started on Day 2 of the administration of the first pharmaceutical composition. In some embodiments, the tacrolimus is started at a dose of 0.015 mg/kg given every 12 hours. In some embodiments, the tacrolimus dose is adjusted to maintain a whole blood 12-hour trough of 3 to 6 ng/mL. In some embodiments, if the subject is also being administered sirolimus the tacrolimus dose is adjusted to maintain a whole blood 12-hour trough of 1-12 ng/ml, 1-10 ng/ml, 1-7 ng/ml, 2-8 ng/mL, 2-7 ng/mL, or 3-6 ng/mL. In some embodiments, if the subject is also being administered sirolimus the tacrolimus dose is adjusted to maintain a whole blood 12-hour trough of 3-6 ng/ml. In some embodiments, if the subject is not also being administered sirolimus, the tacrolimus dose is adjusted to maintain a whole blood 12-hour trough of 6-12 ng/ml, 7-11 ng/ml, or 8-10 ng/mL for the first 1-6 months, 1-4 months, 1-3 months, 2-4 months or 2.5-3.5 months. In some embodiments, if the subject is not also being administered sirolimus, the tacrolimus dose is adjusted to maintain a whole blood 12-hour trough of 8-10 ng/ml for the first 2.5-3.5 months. In some embodiments, 1-10 months 1-8 months, 1-6 months, 3-10 months, 3-8 months, 3-6 months, or 5.5-6.5 months after the subject was administered the composition comprising a population of stem cell-derived islet cells and if the subject is not also being administered sirolimus, the tacrolimus dose is adjusted to maintain a whole blood 12-hour trough of 4-10 ng/ml, 5-9 ng/ml, or 6-8 ng/mL. In some embodiments, 5.5-6.5 months after the subject was administered the composition comprising a population of stem cell-derived islet cells and if the subject is not also being administered sirolimus, the tacrolimus dose is adjusted to maintain a whole blood 12-hour trough of 6-8 ng/mL. In some embodiments, if the subject develops intolerable or clinically undesirable side-effects related to sirolimus or tacrolimus administration, the sirolimus or tacrolimus is replaced with mycophenolate mofetil or mycophenolate sodium. In some embodiments, mycophenolate mofetil is dosed between 500 to 1000 mg twice a day (BID) and mycophenolate sodium is dosed between 360 to 720 mg BID. In some embodiments, the subject is administered 100-5000, 100-3000, 100-2000, 100-1000, 300-5000, 300-2000, 300-1000, 500-1000, 700-1200, 800-1200, 400-900, 400-700 mg of mycophenolate mofetil or mycophenolate sodium. In some embodiments, the mycophenolate mofetil or mycophenolate sodium is administered daily. In some embodiments, the mycophenolate mofetil or mycophenolate sodium is administered orally. In some embodiments, the subject is further administered antimicrobial prophylaxis. In some embodiments, the antimicrobial prophylaxis is trimethoprim, sulfamethoxazole, clotrimazole, valganciclovir, or any combination thereof. In some embodiments, trimethoprim/sulfamethoxazole administration is started after Day 1 of the administration of the first pharmaceutical composition. In some embodiments, clotrimazole administration is started before Day 1 of the administration of the first pharmaceutical composition. In some embodiments, clotrimazole is administered 4 times a day. In some embodiments, the clotrimazole is discontinued 3 months after the administration of the first pharmaceutical composition. In some embodiments, the antimicrobial prophylaxis is valganciclovir, and wherein valganciclovir administration is started before Day 1 of the administration of the first pharmaceutical composition. In some embodiments, the valganciclovir is administered at a dose 450 mg daily (QD) and increases to 900 mg QD by Day 13. In some embodiments, the valganciclovir is administered for at least 14 weeks after the administration of the first pharmaceutical composition. In some embodiments, the subject is administered a second composition comprising a population of cells at a later point in time than the administration of the first composition; wherein the second composition comprises a population of stem cell-derived islet cells. In some embodiments, the second composition is administered to the subject within 30 days of the first composition. In some embodiments, the second composition is administered to the subject at least 3 months, 4 months, 5 months, 6 months, 7 months, 8 months, 9 months, 10 months, 11 months, 12 months, 15 months or between 3-12 months, 3-10 months, 3-9 months, 3-7 months, 3-5 months, 5-12 months, 8-10 months, 7-12 months, 9-15 months, or 9-12 months after the subject is administered the first composition. In some embodiments, the first composition comprises 3.5×10⁸ to about 8.5×10⁸ cells and wherein the second composition comprises 3.5×10⁸ to about 8.5×10⁸ cells. In some embodiments, the first pharmaceutical composition comprises 3.5×10⁸ to about 8.5×10⁸ cells and wherein the second pharmaceutical composition comprises 3.5×10⁸ to about 4.5×10⁸ cells. In some embodiments, the method further comprises administering to the subject at least one immune response modulator before or concurrently with the administration of the second pharmaceutical composition. In some embodiments, the at least one immune response modulator administered before or concurrently with the administration of the second pharmaceutical composition is selected from the group consisting of THYMOGLOBULIN®, Etanercept, Basiliximab, Tacrolimus, Sirolimus, and Mycophenolate mofetil. In some embodiments, the subject is administered Basiliximab. In some embodiments, the method comprises the step of administering an anti-CD25 binding moiety (e.g., basiliximab) concurrently with or after the administration of the second composition; wherein the first and the second composition each comprise a population of cells that express NKX6.1 and ISL1. In some embodiments, the subject is administered multiple doses of basiliximab. In some embodiments, the subject is administered basiliximab on the day the second composition is administered to the subject. In some embodiments, a first administration of Basiliximab administration occurs on the day of administration of the second pharmaceutical (Day A1) followed by a second administration of Basiliximab between: a. Day A2 and Day A8 of the of the administration of the second composition; b. Day A3 and Day A7 of the of the administration of the second composition; and/or c. Day A4 and Day A6 of the of the administration of the second composition. In some embodiments, the second administration of Basiliximab occurs on Day A5 of the of the administration of the second pharmaceutical. In some embodiments, the Basiliximab is administered at a dose of: a. 5 mg to 50 mg; b. 10 mg to 45 mg; c. 15 mg to 40 mg; and/or d. 20 mg to 30 mg. In some embodiments, the Basiliximab is administered at a dose of 20 mg. In some embodiments, the Basiliximab is administered intravenously. In some embodiments, the subject is administered pentoxifylline. In some embodiments, the pentoxifylline administration is started before Day 1 of the administration of the second composition. In some embodiments, the pentoxifylline administration is started on Day −5 to Day −1 of the administration of the second composition. In some embodiments, the pentoxifylline administration is started on Day −2 of the administration of the second composition. In some embodiments, the pentoxifylline is administered at a dose between 250 mg and 500 mg TID. In some embodiments, the pentoxifylline is administered at a dose of 400 mg TID. In some embodiments, the pentoxifylline is administered through at least Day 3 following the administration of the second composition. In some embodiments, the pentoxifylline is administered through at least Day 8 following the administration of the second composition. In some embodiments, the composition comprises about 1×10⁸ to about 7×10⁸, about 1×10⁸ to about 6×10⁸, about 1×10⁸ to about 5×10⁸, about 1×10⁸ to about 4×10⁸, about 1×10⁸ to about 3×10⁸, about 1×10⁸ to about 2×10⁸, about 1.5×10⁸ to about 6.5×10⁸, about 2×10⁸ to about 7×10⁸, about 2×10⁸ to about 6×10⁸, about 2×10⁸ to about 5×10⁸, about 2×10⁸ to about 4×10⁸, about 2×10⁸ to about 3×10⁸, about 2.5×10⁸ to about 5.5×10⁸, about 3×10⁸ to about 7×10⁸, about 3×10⁸ to about 6×10⁸, about 3×10⁸ to about 5×10⁸, about 3×10⁸ to about 4×10⁸, about 3.5×10⁸ to about 4.5×10⁸, or about 3.8×10⁸ to about 4.2×10⁸ cells. In some embodiments, the population of cells are generated from stem cells in vitro. In some embodiments, the composition comprises a plurality of NKX6.1-positive; ISL1-positive cells, wherein the NKX6.1-positive; ISL1-positive cells express higher (e.g., at least 10%, 30%, 50%, 70%, 100%, 125%, 150%, or 200% higher) levels of MAFB, CHGA, ESRRG, SIX2, HOPX, IAPP and/or UCN3 than NKX6.1-positive; ISL1-positive cells from the pancreas of a healthy control adult subject. In some embodiments, the composition comprises a plurality of NKX6.1-positive; ISL1-positive cells, wherein the NKX6.1-positive; ISL1-positive cells express SIX3, MAFA, CHGB, RBP4 and/or FXYD2 at a lower (e.g., at least 10%, 20%, 30%, 40%, 50%, 60%, 70%, 80%, 90% or 100% lower) level than a NKX6.1-positive, ISL1-positive cell from the pancreas of a healthy control adult subject. In some embodiments, the subject received multiple doses of the one or more immunosuppressive agents prior to being administered the composition. In some embodiments, the subject received the one or more immunosuppressive agents for at least 1 month, 2 months, 3 months, 6 months, 9 months, 1 year, 2 years, 3 years, 4 years, 5 years, or 6 years prior to being administered the composition. In some embodiments, the subject received the one or more immunosuppressive agents for 1-12 months, 1-9 months, 1-6 months, 1-3 months, 1-2 months, 1-10 years, 10-20 years, 20-40 years, 1-7 years, 1-5 years, 1-3 years, 1-2 years, or 1-1.5 years prior to being administered the composition. In some embodiments, the subject received the one or more immunosuppressive agents on at least a daily, twice daily, twice weekly, three times a week, weekly, bi-weekly or monthly basis prior to being administered the composition. In some embodiments, the subject is not administered a steroid within 1, 3, 5, 10, 15, 20, 25, 30, 45, 60, 75, or 90 days of being administered the composition. In some embodiments, the subject is not administered prednisone within 1, 3, 5, 10, 15, 20, 25, 30, 45, 60, 75, or 90 days of being administered the composition. In some embodiments, the subject received the organ transplant 0.5-1, 1-2, 3-4, 1-5, 1-10, 3-10, 3-6, 6-10, 10-20, or 20-40 years prior to receiving the composition. In some embodiments, the subject was receiving tacrolimus, sirolimus, mycophenolate mofetil, mycophenolate sodium, and/or etanercept prior to receiving the composition. In some embodiments, the method comprises administering one or more immunosuppressive agents to the subject that are the same as one or more of the immunosuppressive agents administered to the subject prior to receiving the composition. In some embodiments, the method comprises administering the one or more immunosuppressive agents to the subject at the same concentration as the same one or more immunosuppressive agents administered to the subject prior to receiving the composition. In some embodiments, the method comprises administering the one or more immunosuppressive agents to the subject at the same frequency (e.g., daily, every other day, twice weekly, or monthly) as the same one or more immunosuppressive agents administered to the subject prior to receiving the composition. In some embodiments, the transplanted organ is from a human. In some embodiments, the transplanted organ is not an in vitro derived (e.g., in vitro stem cell-derived) organ. In some embodiments, at least 50%, 60%, 70%, 80%, 90%, 93%, 95%, 96%, 97%, 98%, 99% or 100% of the clusters in the composition are less than 200 μm in diameter; or wherein 50-100%, 70-100%, 80-100%, 90-100%, 95-100%, 90-95%, 95-97%, or 97-99%, of the clusters in the composition are less than 200 μm in diameter. In some embodiments, at least 90%, 93%, 95%, 96%, 97%, 98%, 99% or 100% of the clusters in the composition are less than 200 μm in diameter; or wherein 90-100%, 95-100%, 90-95%, 95-97%, or 97-99%, of the clusters in the composition are less than 200 μm in diameter. In some embodiments, at least 50%, 60%, 70%, 80%, 90%, 93%, 95%, 96%, 97%, 98%, 99% or 100% of the clusters in the composition are less than 200 μm in diameter; or wherein 50-100%, 70-100%, 80-100%, 90-100%, 95-100%, 90-95%, 95-97%, or 97-99% of the cells in the composition are in one or more cell clusters. In some embodiments, at least 90%, 93%, 95%, 96%, 97%, 98%, 99% or 100% of the clusters in the composition are less than 200 μm in diameter; or wherein 90-100%, 95-100%, 90-95%, 95-97%, or 97-99% of the cells in the composition are in one or more cell clusters. In some embodiments, the subject did not receive a cadaveric islet transplant prior to receiving the composition. In some embodiments, the transplanted organ is not from a non-human animal (e.g., not from a pig). In some embodiments, the method does not comprise administering tacrolimus, sirolimus, and/or mycophenolate mofetil to the subject. In some embodiments, the subject has Type 1 Diabetes. In some embodiments, the organ transplant comprises an autograft organ transplant. In some embodiments, the organ transplant comprises an allograft organ transplant. In some embodiments, the organ transplant comprises an isograft organ transplant. In some embodiments, the organ transplant comprises a xenograft organ transplant. In some embodiments, the composition comprising the population of stem cell-derived islet cells is administered via the hepatic portal vein. In some embodiments, the composition comprising the population of stem cell-derived islet cells is administered between the rectus sheath and the musculus rectus abdominis. In some embodiments, the composition comprising the population of stem cell-derived islet cells is administered intra-muscularly (e.g., into the forearm). In some embodiments, the composition comprising the population of stem cell-derived islet cells is administered into the peritoneal cavity (e.g., the lesser sac). In some embodiments, the method comprises administering an anti-thymocyte globulin binding moiety in two doses, three doses, four doses, or five doses. In some embodiments, each dose comprises a greater amount of the anti-thymocyte globulin binding moiety than the previous dose. In some embodiments, the anti-thymocyte globulin binding moiety is administered over the course of three doses, and wherein the first dose is 0.25-0.75 mg/kg (e.g., 0.5 mg/kg), the second dose is 0.75-1.25 mg/kg (e.g., 1.0 mg/kg), and the third dose is 1.25-1.75 mg/kg (e.g., 1.5 mg/kg). The details of one or more embodiments of the invention are set forth in the description below. Other features of the present disclosure will be apparent from the following detailed description of several embodiments, and also from the appended claims.

### DETAILED DESCRIPTION

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” means plus or minus 5% of a reference value. For example, “about” 100 refers to 95 to 105.

The term “diabetes” and its grammatical equivalents as used herein can refer to a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some cases, diabetes can be a form of hereditary diabetes.

The term “endocrine cell(s),” if not particularly specified, can refer to hormone-producing cells present in the pancreas of an organism, such as “islet”, “islet cells”, “islet equivalent”, “islet-like cells”, “pancreatic islets” and their grammatical equivalents. In an embodiment, the endocrine cells can be differentiated from pancreatic progenitor cells or precursors. Islet cells can comprise different types of cells, including, but not limited to, pancreatic a cells, pancreatic β cells, pancreatic & cells, pancreatic F cells, and/or pancreatic & cells. Islet cells can also refer to a group of cells, cell clusters, or the like.

As used here, the term “pharmaceutically acceptable” can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C2-C12 alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier” or the like are used interchangeably herein.

The terms “progenitor” and “precursor” cell are used interchangeably herein and refer to cells that have a cellular phenotype that is more primitive (e.g., is at an earlier step along a developmental pathway or progression than is a fully differentiated cell) relative to a cell which it can give rise to by differentiation. Often, progenitor cells can also have significant or very high proliferative potential. Progenitor cells can give rise to multiple distinct differentiated cell types or to a single differentiated cell type, depending on the developmental pathway and on the environment in which the cells develop and differentiate.

A “precursor thereof” as the term related to an insulin-positive endocrine cell can refer to any cell that is capable of differentiating into an insulin-positive endocrine cell, including for example, a pluripotent stem cell, a definitive endoderm cell, a primitive gut tube cell, a pancreatic progenitor cell, or endocrine progenitor cell, when cultured under conditions suitable for differentiating the precursor cell into the insulin-positive endocrine cell.

The terms “stem cell-derived β cell,” “SC-β cell,” “functional β cell,” “functional pancreatic B cell,” “mature SC-β cell,” and their grammatical equivalents can refer to cells (e.g., non-native pancreatic B cells) that display at least one marker indicative of a pancreatic β cell (e.g., PDX-1 or NKX6.1) and expresses insulin. In some embodiments, the SC-β cells display a glucose stimulated insulin secretion (GSIS) response characteristic of an endogenous mature β cell. In some embodiments, the terms “SC-β cell” and “non-native β cell” as used herein are interchangeable. In some embodiments, the “SC-β cell” comprises a mature pancreatic cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the disclosure is not intended to be limited in this manner). In some embodiments, the SC-β cells are generated from embryonic stem cells. In some embodiments, the SC-β cells are generated from induced pluripotent stem cells. In some embodiments, the SC-β cells are generated from induced pluripotent stem cells generated from autologous cells taken from a subject that will be subsequently treated with the SC-β cells. In some embodiments, the SC-β cells are generated from a multipotent stem cell. In some embodiments, the multipotent stem cell is the SR1423 cell line described in Ratiu et al., 2023, bioRxiv, doi.org/10.1101/2023.10.20.563345. In some embodiments, the SC-β cells exhibit a response to multiple glucose challenges (e.g., at least one, at least two, or at least three or more sequential glucose challenges). In some embodiments, the response resembles the response of endogenous islets (e.g., human islets) to multiple glucose challenges. In some embodiments, the morphology of the SC-β cell resembles the morphology of an endogenous B cell. In some embodiments, the SC-β cell exhibits an in vitro GSIS response that resembles the GSIS response of an endogenous B cell. In some embodiments, the SC-β cell exhibits an in vivo GSIS response that resembles the GSIS response of an endogenous β cell. In some embodiments, the SC-β cell exhibits both an in vitro and in vivo GSIS response that resembles the GSIS response of an endogenous B cell. The GSIS response of the SC-β cell can be observed within two weeks of transplantation of the SC-β cell into a host (e.g., a human or animal). In some embodiments, the SC-β cells package insulin into secretory granules. In some embodiments, the SC-β cells exhibit encapsulated crystalline insulin granules. In some embodiments, the SC-β cells exhibit a stimulation index of greater than 1. In some embodiments, the SC-β cells exhibit a stimulation index of greater than 1.1. In some embodiments, the SC-β cells exhibit a stimulation index of greater than 2. In some embodiments, the SC-β cells exhibit cytokine-induced apoptosis in response to cytokines. In some embodiments, insulin secretion from the SC-β cells is enhanced in response to known antidiabetic drugs (e.g., secretagogues). In some embodiments, the SC-β cells are monohormonal. In some embodiments, the SC-β cells do not abnormally co-express other hormones, such as glucagon, somatostatin or pancreatic polypeptide. In some embodiments, the SC-β cells exhibit a low rate of replication. In some embodiments, the SC-β cells increase intracellular Ca2+ in response to glucose. In some embodiments, the SC-β cells express lower levels of MAFA than B cells from the pancreas of a healthy control adult subject. In some embodiments, the SC-β cells express higher levels of MAFB than B cells from the pancreas of a healthy control adult subject. In some embodiments, the SC-β cells express higher levels of SIX2, HOPX, IAPP and/or UCN3 than B cells from the pancreas of a healthy control adult subject. In some embodiments, the SC-β cells do not express MAFA. In some embodiments, the SC-β cells express MAFB. In some embodiments, any of the cell markers disclosed herein (e.g., MAFA, MAFB, SIX2, HOPX, IAPP and/or UCN3) are detected by flow cytometry. In some embodiments, the population comprises one or more NKX6.1-positive, ISL1-positive cells that express CHGA, MAFB, and/or ESRRG at a higher level (e.g., at least 10%, 30%, 50%, 70%, 100%, 125%, 150%, or 200% higher) than a NKX6.1-positive, ISL1-positive cell from the pancreas of a healthy control adult subject. In some embodiments, at least 30%, 40%, 50%, 60%, 70%, 80%, 90%, 95%, or 100% of the NKX6.1-positive, ISL1-positive cells in a population of cells express CHGA, MAFB, and/or ESRRG at a higher level than at least 30%, 40%, 50%, 60%, 70%, 80%, 90%, 95%, or 100% of the NKX6.1-positive, ISL1-positive cells from the pancreas of a healthy control adult subject. In some embodiments, the population comprises one or more NKX6.1-positive, ISL1-positive cells that express SIX3, MAFA, CHGB, RBP4 and/or FXYD2 at a lower level (e.g., at least 10%, 20%, 30%, 40%, 50%, 60%, 70%, 80%, 90% or 100% lower) than a NKX6.1-positive, ISL1-positive cell from the pancreas of a healthy control adult subject. In some embodiments, at least 30%, 40%, 50%, 60%, 70%, 80%, 90%, 95%, or 100% of the NKX6.1-positive, ISL1-positive cells in a population of cells express SIX3, MAFA, CHGB, RBP4 and/or FXYD2 at a lower level than at least 30%, 40%, 50%, 60%, 70%, 80%, 90%, 95%, or 100% of the NKX6.1-positive, ISL1-positive cells from the pancreas of a healthy control adult subject. In some embodiments, the population comprises NKX6.1-positive, ISL1-positive cells that express lower levels of SIX3 than NKX6.1-positive, ISL1-positive cells from the pancreas of a healthy control adult subject. In some embodiments, the population comprises NKX6.1-positive, ISL1-positive cells that express lower levels of CHGB than NKX6.1-positive, ISL1-positive cells from the pancreas of a healthy control adult subject. In some embodiments, the population comprises NKX6.1-positive, ISL1-positive cells that express lower levels of RBP4 than NKX6.1-positive, ISL1-positive cells from the pancreas of a healthy control adult subject. In some embodiments, the population comprises NKX6.1-positive, ISL1-positive cells that express lower levels of FXYD2 than NKX6.1-positive, ISL1-positive cells from the pancreas of a healthy control adult subject.

In some embodiments, any of the NKX6.1-positive, ISL1-positive cells disclosed herein also expresses any one or more of the following genes: PC2, MNX1, or ABCC8.

The terms “stem cell-derived a cell,” “SC-α cell,” “functional a cell,” “functional pancreatic a cell,” “mature SC-α cell,” and their grammatical equivalents can refer to cells (e.g., non-native pancreatic a cells) that display at least one marker indicative of a pancreatic a cell (e.g., glucagon, expressing ISL1 but not NKX6.1), expresses glucagon, and secretes functional glucagon. In some embodiments, the “SC-α cell” does not express somatostatin. In some embodiments, the “SC-α cell” does not express insulin. In some embodiments, the terms “SC-α cell” and “non-native a cell” as used herein are interchangeable. In some embodiments, the “SC-α cell” comprises a mature pancreatic cell.

The terms “stem cell-derived δ cell,” “SC-δ cell,” “functional δ cell,” “functional pancreatic δ cell,” “mature SC-δ cell,” and their grammatical equivalents can refer to cells (e.g., non-native pancreatic δ cells) that display at least one marker indicative of a pancreatic δ cell (e.g., somatostatin), expresses and secretes somatostatin. In some embodiments, “SC-δ cell” does not express glucagon. In some embodiments, “SC-δ cell” does not express insulin. In some embodiments, the terms “SC-δ cell” and “non-native δ cell” as used herein are interchangeable. In some embodiments, the “SC-δ cell” comprises a mature pancreatic cell.

The terms “stem cell-derived enterochromaffin (EC) cell,” “SC-EC cell,” and their grammatical equivalents can refer to cells (e.g., non-native pancreatic EC cells) that display at least one marker indicative of a pancreatic EC cell (e.g., VMAT1, expressing NKX6.1 but not ISL1). In some embodiments, the terms “SC-EC cell” and “non-native EC cell” as used herein are interchangeable.

Similar to SC-β cells, it is to be understood that the SC-α, SC-δ cells, and SC-EC cells need not be derived (e.g., directly) from stem cells, as the methods of the disclosure are capable of deriving SC-α cells from other precursor cells generated during in vitro differentiation of SC-β cells as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the disclosure is not intended to be limited in this manner).

The phrase “stem cell-derived islet cell” or “SC-islet cell” is an islet cell derived from a stem cell. Examples of SC-islet cells include SC-β cells, SC-α, SC-δ cells, and SC-EC cells. SC-islet cells are not mature islet cells obtained from a cadaver or from a human subject. In some embodiments, the SC islet cells are generated from embryonic stem cells. In some embodiments, the SC islet cells are generated from induced pluripotent stem cells. In some embodiments, the SC islet cells are generated from a multipotent stem cell. In some embodiments, the multipotent stem cell is the SR1423 cell line described in Ratiu et al., 2023, bioRxiv, doi.org/10.1101/2023.10.20.563345.

In some embodiments, the induced pluripotent stem cells are generated from mesenchymal stromal cells. In some embodiments, the induced pluripotent stem cells are generated from adipose-derived mesenchymal stromal cells (ADSCs). In some embodiments, the induced pluripotent stem cells are chemically-induced pluripotent stem cells (see, e.g., Guan et al., 2022, Nature, 605:325-331; Wang et al., 2024, Cell, 187, 1-13). In some embodiments, the induced pluripotent stem cells are derived from cells taken from a subject (e.g., a diabetic subject), and the induced pluripotent stem cells are then differentiated in order to make SC-islet cells or precursors thereof that may be administered back to the patient, i.e., the induced pluripotent stem cells are autologous cells to the subject (see, e.g., Wang et al., 2024, Cell, 187, 1-13).

The phrase “therapeutically-effective amount” as used herein in respect to a population of cells means that amount of relevant cells in a population of cells, e.g., SC-β cells or mature pancreatic B cells, or composition comprising SC-β cells of the present disclosure which is effective for producing some desired therapeutic effect in at least a sub-population of cells in an animal at a reasonable benefit/risk ratio applicable to any medical treatment. For example, an amount of a population of SC-β cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, an “immunosuppression-induction agent” is an immunosuppressive agent that may be administered to a subject (e.g., a human subject) as a prophylactic therapy prior to, contemporaneously with, or shortly after transplant of a tissue, organ, or population of cells (e.g., transplant of a kidney or of any of the cells or cell populations disclosed herein) in order to prevent early rejection of the transplanted tissue, organ, or population of cells. Examples of immunosuppression-induction agents are anti-thymocyte globulin binding moieties (e.g., ATG-R, ATG-F, ATGAM), anti CD3 binding moieties (e.g., OKT3), anti-CD25 binding moieties (e.g., daclizumab and basiliximab), or anti-CD52 binding moieties (e.g., alemtuzumab). Immunosuppression-induction agents may also include a high dose of an immunosuppression maintenance agent (e.g., bolus glucocorticosteroids or intravenous cyclosporine [CsA]).

As used herein, an “immunosuppression-maintenance agent” is an immunosuppressive agent that may be administered to a subject (e.g., a human subject) following transplant of a tissue, organ, or population of cells (e.g., transplant of any of the cells or cell populations disclosed herein) for a prolonged period of time or chronically. Examples of maintenance agents include calcineurin inhibitors (e.g., tacrolimus and cyclosporine), antiproliferative agents (e.g., mycophenolate mofetil, mycophenolate sodium and azathioprine), mTOR inhibitors (e.g., sirolimus), and steroids (e.g., prednisone).

As used herein, the term “insulin producing cell” and its grammatical equivalent refer to a cell differentiated from a pancreatic progenitor, or precursor thereof, which secretes insulin. An insulin-producing cell can include pancreatic B cell as that term is described herein, as well as pancreatic β-like cells (e.g., insulin-positive, endocrine cells) that synthesize (e.g., transcribe the insulin gene, translate the proinsulin mRNA, and modify the proinsulin mRNA into the insulin protein), express (e.g., manifest the phenotypic trait carried by the insulin gene), or secrete (release insulin into the extracellular space) insulin in a constitutive or inducible manner. A population of insulin producing cells e.g., produced by differentiating insulin-positive endocrine cells or a precursor thereof into SC-β cells according to the methods of the present disclosure can be pancreatic β cell or (β-like cells (e.g., cells that have at least one, or at least two least two) characteristic of an endogenous β cell and may exhibit a glucose stimulated insulin secretion (GSIS) response that resembles an endogenous adult β cell. The population of insulin-producing cells, e.g., produced by the methods as disclosed herein can comprise mature pancreatic β cell or SC-β cells, and can also contain non-insulin-producing cells (e.g., cells of cell like phenotype with the exception they do not produce or secrete insulin).

The terms “insulin-positive β-like cell,” “insulin-positive endocrine cell,” and their grammatical equivalents can refer to cells (e.g., pancreatic endocrine cells) that display at least one marker indicative of a pancreatic β cell and also expresses insulin but lack a glucose stimulated insulin secretion (GSIS) response characteristic of an endogenous β cell. Exemplary markers of “insulin-positive endocrine cell” include, but are not limited to, NKX6.1, ISL1, and insulin. In some cases, the terms “insulin-positive endocrine cell” and “NKX6.1-positive, ISL1-positive cell” are used interchangeably.

A “cell marker”, as used herein, refers without limitation to proteins, peptides, nucleic acids, polymorphism of proteins and nucleic acids, splice variants, fragments of proteins or nucleic acids, elements, and other analytes which are specifically expressed or present in one or more specific cell types and are not expressed or present in other specific cell types. For example, some of the cells in a population of cells may express PDX1, while other cells in the population do not. In particular embodiments, the presence or absence of one or more cell markers is determined using commercially available antibodies, e.g., in flow cytometry and/or in immunohistochemistry. In other embodiments, the presence or absence of one or more cell markers is detected using RT-PCR. Examples of cell markers include PDX1, NKX6.1, NGN-3, Neuro-D, ISL1, insulin, C-peptide, glucagon, somatostatin, VMAT1, MAFA, and MAFB. Other examples of specific cell markers are referenced throughout this disclosure.

The term “β cell marker” refers to, without limitation, proteins, peptides, nucleic acids, polymorphism of proteins and nucleic acids, splice variants, fragments of proteins or nucleic acids, elements, and other analytes which are specifically expressed or present in pancreatic β cells. Exemplary β cell markers may include, but are not limited to, pancreatic and duodenal homeobox 1 (PDX1) polypeptide, insulin, c-peptide, amylin, E-cadherin, Hnf3β, PCI/3, B2, Nkx2.2, GLUT2, PC2, ZnT-8, ISL1, Pax6, Pax4, NeuroD, 1 Inf1b, Hnf-6, Hnf-3beta, and MafA, and those described in Zhang et al., Diabetes. 50 (10): 2231-6 (2001). In some embodiment, the β cell marker is a nuclear β-cell marker. In some embodiments, the β cell marker is PDX1 or PH3.

The term “pancreatic endocrine marker” can refer to without limitation, proteins, peptides, nucleic acids, polymorphism of proteins and nucleic acids, splice variants, fragments of proteins or nucleic acids, elements, and other analyte which are specifically expressed or present in pancreatic endocrine cells. Exemplary pancreatic endocrine cell markers include, but are not limited to, Ngn-3, NeuroD and Islet-1.

The term “pancreatic progenitor,” “pancreatic endocrine progenitor,” “pancreatic precursor,” “pancreatic endocrine precursor” and their grammatical equivalents are used interchangeably herein and can refer to a stem cell which is capable of becoming a pancreatic hormone expressing cell capable of forming pancreatic endocrine cells, pancreatic exocrine cells or pancreatic duct cells. These cells are committed to differentiating towards at least one type of pancreatic cell, e.g., β cells that produce insulin; a cells that produce glucagon; 8 cells (or D cells) that produce somatostatin; and/or F cells that produce pancreatic polypeptide. Such cells can express at least one of the following markers: NGN3, NKX2.2, NeuroD, ISL-1, Pax4, Pax6, or ARX.

The term “PDX1-positive pancreatic progenitor” or “PDX1-positive, NKX6.1-negative pancreatic progenitor” as used herein can refer to a cell which is a pancreatic endoderm (PE) cell which has the capacity to differentiate into SC-β cells, such as pancreatic B cells. A PDX1-positive pancreatic progenitor expresses the marker PDX1. Other markers include, but are not limited to Cdcp1, or Ptf1a, or HNF6 or NRx2.2. The expression of PDX1 may be assessed by any method known by the skilled person such as flow cytometry, immunochemistry using an anti-PDX1 antibody or quantitative RT-PCR. In some cases, a PDX1-positive pancreatic progenitor cell lacks expression of NKX6.1. In some cases, a PDX1-positive pancreatic progenitor cell can also be referred to as PDX1-positive, NKX6.1-negative pancreatic progenitor cell due to its lack of expression of NKX6.1. In some cases, the PDX1-positive pancreatic progenitor cells can also be termed as “pancreatic foregut endoderm cells.”

The terms “PDX1-positive, NKX6.1-positive pancreatic progenitor,” and “NKX6.1-positive pancreatic progenitor” are used interchangeably herein and can refer to a cell which is a pancreatic endoderm (PE) cell which has the capacity to differentiate into insulin-producing cells, such as pancreatic B cells. A PDX1-positive, NKX6.1-positive pancreatic progenitor expresses the markers PDX1 and NKX6-1. Other markers include, but are not limited to Cdcp1, or Ptf1a, or HNF6 or NRx2.2. The expression of NKX6-1 may be assessed by any method known by the skilled person such as flow cytometry, immunochemistry using an anti-NKX6-1 antibody or quantitative RT-PCR. As used herein, the terms “NKX6.1” and “NKX6-1” are equivalent and interchangeable. In some cases, the PDX1-positive, NKX6.1-positive pancreatic progenitor cells can also be termed as “pancreatic foregut precursor cells.”

The terms “NeuroD” and “NeuroD1” are used interchangeably and identify a protein expressed in pancreatic endocrine progenitor cells and the gene encoding it.

The term “epigenetics” refers to heritable changes in gene function that do not involve changes in the DNA sequence. Epigenetics most often denotes changes in a chromosome that affect gene activity and expression but can also be used to describe any heritable phenotypic change that does not derive from a modification of the genome. Such effects on cellular and physiological phenotypic traits can result from external or environmental factors, or be part of normal developmental program. Epigenetics can also refer to functionally relevant changes to the genome that do not involve a change in the nucleotide sequence. Examples of mechanisms that produce such changes are DNA methylation and histone modification, each of which alters how genes are expressed without altering the underlying DNA sequence. Gene expression can be controlled through the action of repressor proteins that attach to silencer regions of the DNA. These epigenetic changes can last through cell divisions for the duration of the cell's life and can also last for multiple generations even though they do not involve changes in the underlying DNA sequence of the organism. One example of an epigenetic change in eukaryotic biology is the process of cellular differentiation. During morphogenesis, totipotent stem cells become the various pluripotent cells, which in turn can become fully differentiated cells.

The term “epigenetic modifying compound” refers to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, alter DNA accessibility and chromatin structure, thereby regulating patterns of gene expression. These processes are crucial to normal development and differentiation of distinct cell lineages in the adult organism. They can be modified by exogenous influences, and, as such, can contribute to or be the result of environmental alterations of phenotype or pathophenotype. Importantly, epigenetic modification has a crucial role in the regulation of pluripotency genes, which become inactivated during differentiation. Non-limiting exemplary epigenetic modifying compounds include a DNA methylation inhibitor, a histone acetyltransferase inhibitor, a histone deacetylase inhibitor, a histone methyltransferase inhibitor, a bromodomain inhibitor, or any combination thereof.

The term “differentiated cell” or its grammatical equivalents is meant any primary cell that is not, in its native form, pluripotent as that term is defined herein. Stated another way, the term “differentiated cell” can refer to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell may lead to the pancreatic pathway, where, in some embodiments, ~98% of the cells become exocrine, ductular, or matrix cells, and ~2% become endocrine cells. Early endocrine cells are islet progenitors, which can then differentiate further into insulin-producing cells (e.g., functional endocrine cells) which secrete insulin, glucagon, somatostatin, or pancreatic polypeptide. Endoderm cells can also be differentiated into other cells of endodermal origin, e.g., lung, liver, intestine, thymus etc.

As used herein, the term “somatic cell” can refer to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for converting at least one insulin-positive endocrine cell or precursor thereof to an insulin-producing, glucose responsive cell can be performed both in vivo and in vitro (where in vivo is practiced when at least one insulin-positive endocrine cell or precursor thereof are present within a subject, and where in vitro is practiced using an isolated at least one insulin-positive endocrine cell or precursor thereof maintained in culture).

As used herein, the term “adult cell” can refer to a cell found throughout the body after embryonic development.

The term “endoderm cell” as used herein can refer to a cell which is from one of the three primary germ cell layers in the very early embryo (the other two germ cell layers are the mesoderm and ectoderm). The endoderm is the innermost of the three layers. An endoderm cell is capable of differentiating to give rise first to the embryonic gut and then to the linings of the respiratory and digestive tracts (e.g. the intestine), the liver and the pancreas.

The term “a cell of endoderm origin” as used herein can refer to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are capable of developing from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates. Interest in the development and regeneration of the organs has been fueled by the intense need for hepatocytes and pancreatic B cells in the therapeutic treatment of liver failure and type I diabetes. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

The term “definitive endoderm” as used herein can refer to a cell differentiated from an endoderm cell and which can be differentiated into a SC-β cell (e.g., a pancreatic β cell). A definitive endoderm cell expresses the marker Sox17. Other markers characteristic of definitive endoderm cells include, but are not limited to, MIXL2, GATA4, HNF3b, GSC, FGF17, VWF, CALCR, FOXQ1, CXCR4, Cerberus, OTX2, goosecoid, C-Kit, CD99, CMKOR1 and CRIP1. In particular, definitive endoderm cells herein express Sox17 and in some embodiments Sox17 and HNF3B, and do not express significant levels of GATA4, SPARC, APF or DAB. Definitive endoderm cells are not positive for the marker PDX1 (e.g. they are PDX1-negative). Definitive endoderm cells have the capacity to differentiate into cells including those of the liver, lung, pancreas, thymus, intestine, stomach and thyroid. The expression of Sox17 and other markers of definitive endoderm may be assessed by any method known by the skilled person such as immunochemistry, e.g., using an anti-Sox17 antibody, or quantitative RT-PCR.

The term “pancreatic endoderm” can refer to a cell of endoderm origin which is capable of differentiating into multiple pancreatic lineages, including pancreatic B cells, but no longer has the capacity to differentiate into non-pancreatic lineages.

The term “primitive gut tube cell” or “gut tube cell” as used herein can refer to a cell differentiated from an endoderm cell and which can be differentiated into a SC-β cell (e.g., a pancreatic β cell). A primitive gut tube cell expresses at least one of the following markers: HNP1-β, HNF3-β or HNF4-α. In some cases, a primitive gut tube cell is FOXA2-positive and SOX2-positive, i.e., express both FOXA2 (also known as HNF3-β) and SOX2. In some cases, a primitive gut tube cell is FOXA2-positive and PDX1-negative, i.e., express FOXA2 but not PDX1. Primitive gut tube cells have the capacity to differentiate into cells including those of the lung, liver, pancreas, stomach, and intestine. The expression of HNF1-β and other markers of primitive gut tube may be assessed by any method known by the skilled person such as immunochemistry, e.g., using an anti-HNF1-β antibody.

The term “stem cell” as used herein, can refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” can refer to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retro-differentiation” by persons of ordinary skill in the art. As used herein, the term “pluripotent stem cell” includes embryonic stem cells, induced pluripotent stem cells, placental stem cells, etc.

The term “pluripotent” as used herein can refer to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and can refer to a pluripotent stem cell artificially derived (e.g., induced or by complete reversal) from a non-pluripotent cell, typically an adult somatic cell, for example, by inducing a forced expression of one or more genes.

The term “phenotype” can refer to one or a number of total biological characteristics that define the cell or organism under a particular set of environmental conditions and factors, regardless of the actual genotype.

The terms “subject,” “patient,” or “individual” are used interchangeably herein, and can refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject can refer to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g. cow, sheep, pig, and the like. “Patient in need thereof” or “subject in need thereof” is referred to herein as a patient diagnosed with or suspected of having a disease or disorder, for instance, but not restricted to diabetes.

“Administering” used herein can refer to providing one or more compositions described herein (e.g., a pharmaceutically acceptable composition comprising any of the cells or populations of cells disclosed herein) to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In some embodiments, administration is administration of one or more devices housing a cell population into a subject (e.g., subcutaneously and/or preperitoneally). In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, any of the cell compositions disclosed herein may be administered by intravenous (i.v.) injection, sub-cutaneous (s.c.) injection, intradermal (i.d.) injection, intraperitoneal (i.p.) injection, or intramuscular (i.m.) injection. In particular embodiments, the cell composition is administered via the hepatic portal vein. See, e.g., Reichman et al., NEJM, 2025, DOI: 10.1056/NEJMoa2506549, which is incorporated by reference herein in its entirety. In other embodiments, the cell composition is administered under the Anterior Rectus Sheath, e.g., within a space between the rectus sheath and the musculus rectus abdominis. See, e.g., WO2023227068, which is incorporated by reference herein in its entirety. In some embodiments, the cell composition is administered intramuscularly, e.g., into a forearm of a subject. In some embodiments, the cell composition is administered into the peritoneal cavity (e.g., the lesser sac). In some embodiments, the cell composition is administered to the subject in a device. In some embodiments, the device encapsulates the cells and is porous and permits insulin to exit the device and nutrients to enter the device, but prevents the islet cells from exiting the device or immune cells (e.g., T cells or macrophages) from entering the device. In some embodiments, the cell composition is administered to the subject in a multilayer lattice structure, e.g., an alginate structure (see, e.g., WO2023220816 and WO2023220817, which are incorporated herein in their entirety).

### Overview

Disclosed herein are methods of treating a subject having diabetes. The methods can comprise treating the subject with a composition comprising a population of stem cell-derived islet cells, and one or more immunosuppressive agents. The methods can comprise administering a composition comprising a population of stem cell-derived islet cells, and one or more immunosuppressive agents to the subject. In some examples, the subject (e.g., the diabetic subject) previously received an organ transplant. In some examples, the method includes selecting a subject (e.g., a diabetic subject) who previously received an organ transplant. In some examples, the subject (e.g., the diabetic subject) previously received a kidney transplant. In some examples, the method includes selecting a subject (e.g., a diabetic subject) who previously received a kidney transplant. In some examples, the subject (e.g., the diabetic subject) previously received a liver transplant. In some examples, the method includes selecting a subject (e.g., a diabetic subject) who previously received a liver transplant. In some examples, the composition used in the treatment comprises about 1×10⁸ to about 10×10⁸ cells. In some examples, the composition used in the treatment comprises a plurality of C-peptide-positive, ISL1-positive cells. In some examples, the composition used in the treatment comprises a plurality of NKX6.1-positive, ISL1-positive cells.

In some examples 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, or 80-90% of the cells in the composition used in the treatment express C-peptide and ISL1. In some examples 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the composition used in the treatment express glucagon but not somatostatin. In some examples, 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the composition used in the treatment express somatostatin but not glucagon. In particular examples, 35-65% of the cells in the composition used in the treatment express C-peptide and ISL1. In further particular examples 4-25%, of the cells in the composition used in the treatment express glucagon but not somatostatin. In additional particular examples, 1-10% of the cells in the composition used in the treatment express somatostatin but not glucagon.

In some examples, less than 40%, less than 35%, less than 30%, less than 25%, less than 20%, less than 18%, less than 15%, less than 12%, or less than 10% of the cells in the composition used in the treatment express VMAT1 but not C-peptide. In some examples, no less than 50%, 40%, 30%, or 20% of the cells in the composition used in the treatment are NKX6.1⁺/ISL1⁺ cells. In particular examples, no less than 20% of the cells in the composition used in the treatment are NKX6.1⁺/ISL1⁺ cells. In some examples, no less than 40%, 35%, 30%, 26%, 25%, 20%, 15% or 10% of the cells in the composition used in the treatment are NKX6.1⁻/ISL1⁺ cells. In some examples, between 5-25%, 5-40%, 5-35%, or 8-20% of the cells in the composition used in the treatment are NKX6.1⁻/ISL1⁺ cells. In some examples, no more than 50%, 45%, 40%, 35%, 30%, or 25% of the cells in the composition used in the treatment are NKX6.1⁺/ISL1⁻ cells. In some examples, between 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, 40-50% of the cells in the composition used in the treatment are NKX6.1⁺/ISL1⁺ cells. In some examples, between 8-20%, 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, or 40-50% of the cells in the composition used in the treatment are NKX6.1⁻/ISL1⁺ cells. In some examples, between 3-50%, 3-40%, 3-30%, 3-25%, 3-20%, 3-15%, 3-10%, or 3-5% of the cells in the composition used in the treatment are NKX6.1⁺/ISL1⁻ cells. In some examples, less than 6% of the cells in the composition used in the treatment are NKX6.1⁻/ISL1⁻ cells. In some examples, between 0-6%, 0-4%, 0-2%, 0-1%, 1-6%, 1-5%, 1-4%, 1-3%, 1-2%, 2-3%, or 3-6% of the cells in the composition used in the treatment are NKX6.1⁻/ISL1⁻ cells. In some particular examples, at least 30% of the cells in the composition used in the treatment are NKX6.1-positive, ISL1-positive cells. In further particular examples, at least 15% of the cells in the composition used in the treatment are NKX6.1-negative, ISL1-positive cells. In additional particular examples, less than 12% of the cells in the composition used in the treatment are NKX6.1-negative, ISL1-negative cells. In some particular examples, less than 20% of the cells in the composition used in the treatment are NKX6.1-positive, ISL1-negative cells.

In some examples, the composition used in the treatment comprises NKX6.1⁺/ISL1⁺ cells that display a glucose stimulated insulin secretion (GSIS) response in vitro. In some examples, the composition used in the treatment comprises NKX6.1⁺/ISL1⁺ cells that display a glucose stimulated insulin secretion (GSIS) response in vivo.

In some examples, a plurality of cells in the composition used in the treatment are in one or more cell clusters. In some examples, the one or more cell clusters are about 50 μm to about 500 μm, about 50 μm to about 400 μm, about 50 μm to about 300 μm, about 60 μm to about 400 μm, about 60 μm to about 300 μm, about 60 μm to about 250 μm, about 75 μm to about 400 μm, about 75 μm to about 300 μm, about 75 μm to about 250 μm, about 125 μm to about 225 μm, about 130 μm to about 160 μm, about 170 μm to about 225 μm, about 140 μm to about 200 μm, about 140 μm to about 170 μm, about 160 μm to about 220 μm, about 170 μm to about 215 μm, or about 170 μm to about 200 μm in diameter. In some examples, the composition used in the treatment does not comprise any clusters greater than 200, 250, 300, 400, 450, or 500 μm in diameter. In some embodiments, at least 40%, 50%, 60%, 70%, 80%, 90%, 95%, 98%, or 100% of the cells in the composition are in one or more cell clusters. In some embodiments, 40-100%, 40-60%, 60-100%, 60-80%, 80-100%, 70-95%, 80-90%, 60-90%, 90-98%, or 90-100% of the cells in the composition are in one or more cell clusters. In some embodiments, the cell clusters comprise at least 10 cells. In some embodiments, at least 20%, 40%, 50%, 60%, 70%, 80%, 90%, 95%, 98%, or 100% of the cells in the composition are dissociated. In some embodiments, 20-40%, 40-100%, 40-60%, 60-100%, 60-80%, 80-100%, 70-95%, 80-90%, 60-90%, 90-98%, or 90-100% of the cells in the composition are dissociated.

In some examples, the one or more immunosuppressive agents are an anti-thymocyte globulin binding moieties (e.g., ATG-R, ATG-F, ATGAM), an anti-CD3 binding moiety (e.g., OKT3), an anti-CD25 binding moiety (e.g., daclizumab or basiliximab), an anti-CD52 binding moiety (e.g., alemtuzumab), or a high dose of an immunosuppression maintenance agent (e.g., bolus glucocorticosteroids or intravenous cyclosporine [CsA]). In some particular examples, the immunosuppressive agent is an anti-thymocyte globulin binding moiety. In some examples, the method comprises administering multiple doses of the one or more immunosuppressive agents. In some examples, the method comprises administering multiple doses of the one or more immunosuppressive agents in a single day. In some examples, the method comprises administering multiple doses of one or more immunosuppression-induction agents over several days. In some examples, a dose of the one or more immunosuppressive agents is administered at 0.05-3 mg/kg. In some examples, the subject is treated with a Tumor Necrosis Factor-alpha (TNFα) inhibitor. In some particular examples, the method comprises treating the subject with 10-100 mg of a Tumor Necrosis Factor-alpha (TNFα) inhibitor. In some examples, the method comprises treating the subject with sirolimus (e.g., 0.01-5000 mg/kg of sirolimus). In some examples, the method comprises treating the subject with tacrolimus (e.g., 0.001-0.05 mg/kg of tacrolimus). In some examples, the method comprises treating the subject with mycophenolate mofetil. In some examples, the method comprises treating the subject with mycophenolate sodium. In particular examples, the method comprises treating the subject with 100-5000 mg of mycophenolate mofetil. In particular examples, the method comprises treating the subject with 100-5000 mg of mycophenolate sodium. In some examples, the subject is treated with an antimicrobial agent.

In some examples, the method further comprises administering to the subject a second composition comprising a second population of cells. In some examples, the method further comprises administering to the subject an anti-CD25 binding moiety (e.g., basiliximab) concurrently with or after the administration of the second composition. In some examples, the second composition comprises a plurality of cells that express ISL1. In some particular examples, the method further comprises i) administering to the subject a second composition comprising a second population of cells, and ii) administering to the subject an anti-CD25 binding moiety (e.g., basiliximab) concurrently with or after the administration of the second composition, wherein the second composition comprises a plurality of cells that express ISL1.

In some examples, the method further comprises administering to the subject a second composition comprising a second population of cells. In some examples, the method further comprises administering to the subject a vasoactive agent (e.g., pentoxifylline) concurrently with or after the administration of the second composition. In some examples, the second composition comprises a population of cells that express ISL1. In some particular examples, the method further comprises i) administering to the subject a second composition comprising a second population of cells, and ii) administering to the subject a vasoactive agent (e.g., pentoxifylline) concurrently with or after the administration of the second composition, wherein the second composition comprises a population of cells that express ISL1.

In some examples, the subject is administered a first dose of 0.05-3 mg/kg anti-thymocyte globulin binding moiety, such as within 72-24 hours prior to being treated with the composition. In particular examples, the anti-thymocyte globulin moiety is administered via infusion. In particular examples the infusion is via a high-flow vein. In some examples, the infusion lasts for 2-20, 3-18, 4-15, 5-13, 6-12, 7-11, 8-10, or 9-11 hours. In some examples, the subject is not administered any anti-thymocyte globulin binding moiety for a resting period between doses. In some examples, the resting period is 1-20, 6-15, 12-24, 10-20, 1-12, 2-11, 3-10, 4-9, 5-8, 5-7, 5-15, 5-20, 8-10, or 9-14 hours. In some examples, the first dose of the anti-thymocyte globulin binding moiety administration protocol is infused over a period of 6-12 hours. In some examples, any subsequent dose of the anti-thymocyte globulin binding moiety administration protocol, if any, is infused over a period of 6-12 hours. In some examples, there are at least 6 hours between the end of one anti-thymocyte globulin binding moiety infusion and the start of a subsequent anti-thymocyte globulin binding moiety infusion. In particular examples, the total dose of anti-thymocyte globulin binding moiety at the end of the anti-thymocyte globulin binding moiety administration protocol is between 1 mg/kg to 10 mg/kg. In particular examples, the total dose of anti-thymocyte globulin binding moiety at the end of the anti-thymocyte globulin binding moiety administration protocol is between 2 mg/kg to 9 mg/kg. In particular examples, the total dose of anti-thymocyte globulin binding moiety at the end of the anti-thymocyte globulin binding moiety administration protocol is between 3 mg/kg to 8 mg/kg. In particular examples, the total dose of anti-thymocyte globulin binding moiety at the end of the anti-thymocyte globulin binding moiety administration protocol is between 4 mg/kg to 7 mg/kg. In particular examples, the total dose of anti-thymocyte globulin binding moiety at the end of the anti-thymocyte globulin binding moiety administration protocol is between 5 mg/kg to 6 mg/kg. In particular examples, the total dose of the anti-thymocyte globulin binding moiety administration is 6 mg/kg. In some examples, the anti-thymocyte globulin binding moiety administration protocol includes dosing the first dose of anti-thymocyte globulin binding moiety administration at a lower dose than any subsequent dose of anti-thymocyte globulin binding moiety.

In some examples, the anti-thymocyte globulin binding moiety administration protocol includes monitoring of total white blood cell (WBC) and platelet counts of the subject following a dose of the anti-thymocyte globulin binding moiety. In some examples, the anti-thymocyte globulin binding moiety administration protocol includes reducing a subsequent dose of anti-thymocyte globulin binding moiety (e.g., by at least 10%, 20%, 30%, 40%, 50%, 60%, 70%, 80%, 90% or 100%) if the WBC count is between 2,000 and 3,000 cells/mm³. In some examples, the anti-thymocyte globulin binding moiety administration protocol includes reducing a subsequent dose of anti-thymocyte globulin binding moiety (e.g., by at least 10%, 20%, 30%, 40%, 50%, 60%, 70%, 80%, 90% or 100%) if the platelet count is between 50,000 and 75,000 cells/mm³. In particular examples, the anti-thymocyte globulin binding moiety administration protocol includes monitoring of total white blood cell (WBC) and platelet counts of the subject following a dose of the anti-thymocyte globulin binding moiety and reducing a subsequent dose of anti-thymocyte globulin binding moiety (e.g., by at least 10%, 20%, 30%, 40%, 50%, 60%, 70%, 80%, 90% or 100%) if (a) the WBC count is between 2,000 and 3,000 cells/mm³ and/or (b) the platelet count is between 50,000 and 75,000 cells/mm³. In some examples, the anti-thymocyte globulin binding moiety administration protocol includes monitoring of total white blood cell (WBC) and/or platelet counts of the subject following a dose of the anti-thymocyte globulin binding moiety. In some examples, the anti-thymocyte globulin binding moiety administration protocol includes stopping the anti-thymocyte globulin binding moiety administration if the WBC count falls below 2,000 cells/mm³. In some examples, the anti-thymocyte globulin binding moiety administration protocol includes stopping the anti-thymocyte globulin binding moiety administration if the platelet count falls below 50,000 cells/mm³. In particular examples, the anti-thymocyte globulin binding moiety administration protocol includes monitoring of total white blood cell (WBC) and/or platelet counts of the subject following a dose of the anti-thymocyte globulin binding moiety and stopping the anti-thymocyte globulin binding moiety administration if (a) the WBC count falls below 2,000 cells/mm³ and/or (b) the platelet count falls below 50,000 cells/mm³.

In some examples, the subject is treated with 2, 3, 4, 5, 6, or 7 doses of the TNFα inhibitor in the month following the administration of the composition comprising the population of cells. In some examples, the subject is treated with the TNFα inhibitor about 3 days, about 4 days, about 5 days, about 6 days, about 7 days, about 8 days, about 9 days, about 10 days, about 11 days, about 12 days, about 13 days, about 14 days, and/or about 15 days following the administration of the composition. In some examples, the subject is treated with 5-500, 5-100, 5-75, 5-50, 10-100, 10-75, 10-50, 30-100, or 30-75 mg TNFα inhibitor for each dose of the TNFα inhibitor. In some examples, the TNFα inhibitor is administered subcutaneously. In some examples, the TNFα inhibitor is administered intravenously. In some examples, the TNFα inhibitor comprises etanercept, adalimumab, infliximab, certolizumab pegol, and/or golimumab.

In some examples, the subject is administered etanercept. In some examples, etanercept is administered on the same day as the administration of the composition comprising the population of cells. In some examples, etanercept is administered on the same day as the administration of the composition comprising the population of cells (Day 1) followed by further administration on Day 2, Day 3, Day 4, Day 5, Day 6, Day 7, Day 8, Day 9, Day 10, Day 11, Day 12, Day 13, Day 14, and/or Day 15. In some examples, etanercept is administered on multiple days starting on the same day as the administration of the composition. In some examples, a first administration of etanercept occurs on the same day as the administration of the composition (Day 1) followed by an administration between Day 2 to Day 6. In some examples, a first administration of etanercept occurs on the same day as the administration of the composition (Day 1) followed by an administration between Day 5 to Day 10. In some examples, a first administration of etanercept occurs on the same day as the administration of the composition (Day 1) followed by an administration between Day 9 to Day 14. In some examples, etanercept is not administered on consecutive days. In particular examples, a first administration of etanercept occurs on the same day as the administration of the composition (Day 1) followed by (a) a second administration between Day 2 to Day 6, (b) a third administration between Day 5 to Day 10, and/or (c) a fourth administration between Day 9 to Day 14, optionally wherein etanercept is not administered on consecutive days. In some examples, a first administration of etanercept occurs on the same day as the administration of the composition (Day 1) followed by an administration between Day 3 to Day 5. In some examples, a first administration of etanercept occurs on the same day as the administration of the composition (Day 1) followed by an administration between Day 7 to Day 9. In some examples, a first administration of etanercept occurs on the same day as the administration of the composition (Day 1) followed by an administration between Day 10 to Day 12. In particular examples, a first administration of etanercept occurs on the same day as the administration of the composition (Day 1) followed by (a) a second administration between Day 3 to Day 5, (b) a third administration between Day 7 to Day 9, and/or (c) a fourth administration between Day 10 to Day 12. In some examples, a first administration of etanercept occurs on the same day as the administration of the composition (Day 1) followed by an administration on day 4. In some examples, a first administration of etanercept occurs on the same day as the administration of the composition (Day 1) followed by an administration on day 8. In some examples, a first administration of etanercept occurs on the same day as the administration of the composition (Day 1) followed by an administration on day 11. In some examples, a first administration of etanercept occurs on the same day as the administration of the composition (Day 1) followed by (a) a second administration on Day 4, (b) a third administration on Day 8, and/or (c) a fourth administration on Day 11.

In some examples, etanercept is administered at a dose of 10 mg to 100 mg. In some examples, etanercept is administered at a dose of 15 mg to 95 mg. In some examples, etanercept is administered at a dose of 20 mg to 90 mg. In some examples, etanercept is administered at a dose of 25 mg to 85 mg. In some examples, etanercept is administered at a dose of 30 mg to 80 mg. In some examples, etanercept is administered at a dose of 35 mg to 75 mg. In some examples, etanercept is administered at a dose of 40 mg to 70 mg. In some examples, etanercept is administered at a dose of 45 mg to 65 mg. In some examples, etanercept is administered at a dose of 50 mg to 60 mg. In some examples, etanercept is administered at a dose of 50 mg on the same day as the administration of the composition (Day 1). In some examples, etanercept is administered at a dose of 25 mg for any administration occurring on Day 2 or after.

In some examples, etanercept administration is intravenous (IV). In some examples, etanercept administration is subcutaneous. In some examples, the first etanercept administration is a different administration method than subsequent etanercept administrations. In particular examples, the first etanercept administration is IV and the subsequent etanercept administrations are subcutaneous.

In some examples, prior to the administration of the composition comprising the population of cells, the subject received infusion of insulin at a level of at least about 20 U/day, 22 U/day, 24 U/day, 26 U/day, 28 U/day, 30 U/day, 32 U/day, and/or 34 U/day. In some examples, the subject is treated with 0.01-1, 0.02-0.3, 0.02-0.15, 0.02-0.12, 0.02-0.08, 0.03-0.07, 0.04-0.15, and/or 0.05-2 mg/kg sirolimus. In some examples, the sirolimus is administered daily. In some examples, the sirolimus is administered orally. In some examples, the subject is treated with 0.001-0.05, 0.003-0.03, 0.004-0.02, 0.006-0.02, 0.008-0.02, 0.01-0.02, 0.009-0.03, and/or 0.009-0.02 mg/kg tacrolimus. In some examples, the tacrolimus is administered daily. In some examples, the tacrolimus is administered orally. In some examples, the first dose of Sirolimus is 0.05 to 0.2 mg/kg. In some examples, subsequent doses of Sirolimus are 0.05 to 0.1 mg/kg once each morning (qam). In particular examples, the first dose of Sirolimus is 0.05 to 0.2 mg/kg and subsequent doses are 0.05 to 0.1 mg/kg qam. In some examples, the sirolimus dose is adjusted to maintain a whole blood 24-hour trough target of 10 to 15 ng/mL for the first 3 months after administration of the composition. In some examples, the sirolimus dose is adjusted to maintain a whole blood 24-hour trough target of 8 to 12 ng/mL after the first 3 months after administration of the composition. In some examples, the administration of tacrolimus is started after the day of the administration of the composition comprising the population of cells (Day 1). In some examples, the administration of tacrolimus is started between Day 2 and Day 5 of the administration of the composition. In some examples, the administration of tacrolimus is started on Day 2 of the administration of the first pharmaceutical composition. In some examples, the tacrolimus is started at a dose of 0.015 mg/kg given every 12 hours. In some examples, the tacrolimus dose is adjusted to maintain a whole blood 12-hour trough of 3 to 6 ng/mL. In some examples, if the subject develops intolerable or clinically undesirable side-effects related to sirolimus or tacrolimus treatment, the sirolimus or tacrolimus is replaced with mycophenolate mofetil and/or mycophenolate sodium. In some examples, mycophenolate mofetil is dosed between 500 to 1000 mg twice a day (BID). In some examples mycophenolate sodium is dosed between 360 to 720 mg BID. In particular examples, mycophenolate mofetil is dosed between 500 to 1000 mg BID and mycophenolate sodium is dosed between 360 to 720 mg BID. In some examples, the subject is treated with 100-5000, 100-3000, 100-2000, 100-1000, 300-5000, 300-2000, 300-1000, 500-1000, 700-1200, 800-1200, 400-900, 400-700 mg of mycophenolate mofetil or mycophenolate sodium. In some examples, the mycophenolate mofetil and/or mycophenolate sodium is administered daily. In some examples, the mycophenolate mofetil and/or mycophenolate sodium is administered orally.

In some examples, the subject is further administered antimicrobial prophylaxis. In some examples, the antimicrobial prophylaxis is trimethoprim, sulfamethoxazole, clotrimazole, valganciclovir, or any combination thereof. In some examples, trimethoprim/sulfamethoxazole administration is started after Day 1 of the administration of the first pharmaceutical composition. In some examples, clotrimazole administration is started before Day 1 of the administration of the first pharmaceutical composition. In some examples, clotrimazole is administered 4 times a day. In some examples, the clotrimazole is discontinued 3 months after the administration of the first pharmaceutical composition. In some examples, valganciclovir administration is started before Day 1 of the administration of the first pharmaceutical composition. In some examples, the valganciclovir is administered at a dose 450 mg daily (QD). In some examples, valganciclovir administration increases to 900 mg QD by Day 13. In particular examples, the valganciclovir is administered at a dose 450 mg QD and increases to 900 mg QD by Day 13. In some examples, the valganciclovir is administered for at least 14 weeks after the administration of the first pharmaceutical composition.

In some examples, the subject is administered a second composition comprising a population of cells at a later point in time than the administration of the first composition. In some examples, the second composition comprises a population of stem cell-derived islet cells. In some examples, the second composition is administered to the subject within 30 days of the first composition. In some examples, the second composition is administered to the subject at least 3 months, 4 months, 5 months, 6 months, 7 months, 8 months, 9 months, 10 months, 11 months, 12 months, 15 months after the subject is administered the first composition. In some examples, the second composition is administered to the subject between 3-12 months, 3-10 months, 3-9 months, 3-7 months, 3-5 months, 5-12 months, 8-10 months, 7-12 months, 9-15 months, or 9-12 months after the subject is administered the first composition. In some examples, the first composition comprises 3.5×10⁸ to about 8.5×10⁸ cells and wherein the second composition comprises 3.5×10⁸ to about 8.5×10⁸ cells. In particular examples, the first pharmaceutical composition comprises 3.5×10⁸ to about 8.5×10⁸ cells and wherein the second pharmaceutical composition comprises 3.5×10⁸ to about 4.5×10⁸ cells. In some examples, the method further comprising administering to the subject at least one immune response modulator before or concurrently with the administration of the second pharmaceutical composition. In some examples, the at least one immune response modulator administered before or concurrently with the administration of the second pharmaceutical composition is selected from the group consisting of THYMOGLOBULIN®, Etanercept, Basiliximab, Tacrolimus, Sirolimus, and Mycophenolate mofetil. In some examples, the at least one immune response modulator administered before or concurrently with the administration of the second pharmaceutical composition comprises THYMOGLOBULIN®, Etanercept, Basiliximab, Tacrolimus, Sirolimus, and/or Mycophenolate mofetil. In particular examples, the subject is administered Basiliximab.

In some examples, the method comprises administering an anti-CD25 binding moiety. In some examples, the method comprises administering an anti-CD25 binding moiety (e.g., basiliximab) concurrently with or after the administration of the second composition, wherein the first and the second composition each comprise a population of cells that express NKX6.1 and ISL1. In some examples, the subject is administered multiple doses of basiliximab. In some examples, the subject is administered basiliximab on the day the second composition is administered to the subject. In some examples, a first administration of basiliximab administration occurs on the day of administration of the second pharmaceutical (Day A1) followed by an administration of basiliximab between Day A2 and Day A8 of the of the administration of the second composition. In some examples, a first administration of basiliximab administration occurs on the day of administration of the second pharmaceutical (Day A1) followed by an administration of basiliximab between Day A3 and Day A7 of the of the administration of the second composition. In some examples, a first administration of basiliximab administration occurs on the day of administration of the second pharmaceutical (Day A1) followed by an administration of Basiliximab between Day A4 and Day A6 of the of the administration of the second composition. In some examples, a first administration of Basiliximab administration occurs on the day of administration of the second pharmaceutical (Day A1) followed by a second administration of Basiliximab between (a) Day A2 and Day A8 of the of the administration of the second composition, (b) Day A3 and Day A7 of the of the administration of the second composition, and/or (c) Day A4 and Day A6 of the of the administration of the second composition. In some examples, an administration of Basiliximab occurs on Day A5 of the of the administration of the second pharmaceutical. In particular examples, the second administration of Basiliximab occurs on Day A5 of the of the administration of the second pharmaceutical. In some examples, the Basiliximab is administered at a dose of 5 mg to 50 mg. In some examples, the Basiliximab is administered at a dose of 10 mg to 45 mg. In some examples, the Basiliximab is administered at a dose of 15 mg to 40 mg. In some examples, the Basiliximab is administered at a dose of 20 mg to 30 mg. In particular examples, the Basiliximab is administered at a dose of 20 mg. In some examples, the Basiliximab is administered intravenously.

In some examples, the subject is administered pentoxifylline. In some examples, the pentoxifylline administration is started before Day 1 of the administration of the second composition. In some examples, the pentoxifylline administration is started on Day −5 to Day −1 of the administration of the second composition. In some examples, the pentoxifylline administration is started on Day −2 of the administration of the second composition. In some examples, the pentoxifylline is administered at a dose between 250 mg and 500 mg TID. In particular examples, the pentoxifylline is administered at a dose of 400 mg TID. In some examples, the pentoxifylline is administered through at least Day 3 following the administration of the second composition. In some examples, the pentoxifylline is administered through at least Day 8 following the administration of the second composition.

In some examples, the composition used in the treatment comprises about 1×10⁸ to about 7×10⁸, about 1×10⁸ to about 6×10⁸, about 1×10⁸ to about 5×10⁸, about 1×10⁸ to about 4×10⁸, about 1×10⁸ to about 3×10⁸, about 1×10⁸ to about 2×10⁸, about 1.5×10⁸ to about 6.5×10⁸, about 2×10⁸ to about 7×10⁸, about 2×10⁸ to about 6×10⁸, about 2×10⁸ to about 5×10⁸, about 2×10⁸ to about 4×10⁸, about 2×10⁸ to about 3×10⁸, about 2.5×10⁸ to about 5.5×10⁸, about 3×10⁸ to about 7×10⁸, about 3×10⁸ to about 6×10⁸, about 3×10⁸ to about 5×10⁸, about 3×10⁸ to about 4×10⁸, about 3.5×10⁸ to about 4.5×10⁸, or about 3.8×10⁸ to about 4.2×10⁸ cells. In particular examples, the composition used in the treatment comprises about 3.8×10⁸ to about 4.2×10⁸ cells. In some examples, the population of cells are generated from stem cells in vitro. In some examples, the composition used in the treatment comprises a plurality of NKX6.1-positive; ISL1-positive cells, wherein the NKX6.1-positive; ISL1-positive cells express higher (e.g., at least 10%, 30%, 50%, 70%, 100%, 125%, 150%, or 200% higher) levels of MAFB, CHGA, ESRRG, SIX2, HOPX, IAPP and/or UCN3 than control cells. In some examples, the control cells are NKX6.1-positive; ISL1-positive cells from the pancreas of a healthy control adult subject. In some examples, the composition used in the treatment comprises a plurality of NKX6.1-positive; ISL1-positive cells, wherein the NKX6.1-positive; ISL1-positive cells express SIX3, MAFA, CHGB, RBP4 and/or FXYD2 at a lower (e.g., at least 10%, 20%, 30%, 40%, 50%, 60%, 70%, 80%, 90% or 100% lower) level than control cells. In some examples, the control cells are NKX6.1-positive, ISL1-positive cell from the pancreas of a healthy control adult subject.

In some examples, the subject received multiple doses of immunosuppressive agents prior to being administered the composition. In some embodiments, the subject has been receiving one or more immunosuppressive agents as a result of a prior organ transplant (e.g., a prior kidney or liver transplant). In some examples, the method includes selecting a subject who received multiple doses of immunosuppressive agents. In some examples, the subject received one or more immunosuppressive agents for at least 1 month, 2 months, 3 months, 6 months, 9 months, 1 year, 2 years, 3 years, 4 years, 5 years, or 6 years prior to being administered the composition. In some examples, the method includes selecting a subject who has received one or more immunosuppressive agents for at least 1 month, 2 months, 3 months, 6 months, 9 months, 1 year, 2 years, 3 years, 4 years, 5 years, or 6 years. In some examples, the subject received immunosuppressive agents for 1-12 months, 1-9 months, 1-6 months, 1-3 months, 1-2 months, 1-10 years, 10-20 years, 20-40 years, 1-7 years, 1-5 years, 1-3 years, 1-2 years, or 1-1.5 years prior to being administered the composition. In some examples, the method includes selecting a subject who received immunosuppressive agents for 1-12 months, 1-9 months, 1-6 months, 1-3 months, 1-2 months, 1-10 years, 10-20 years, 20-40 years, 1-7 years, 1-5 years, 1-3 years, 1-2 years, or 1-1.5 years. In some examples, the subject received immunosuppressive agents on at least a daily, twice daily, twice weekly, three times a week, weekly, bi-weekly or monthly basis prior to being administered the composition. In some examples, the method includes selecting a subject who received immunosuppressive agents on at least a daily, twice daily, twice weekly, three times a week, weekly, bi-weekly or monthly basis. In some examples, the subject is not administered a steroid within 1, 3, 5, 10, 15, 20, 25, 30, 45, 60, 75, or 90 days of being administered the composition. In some examples, the method includes selecting a subject who has not received a steroid within the past 1, 3, 5, 10, 15, 20, 25, 30, 45, 60, 75, or 90 days. In some examples, the subject is not administered prednisone within 1, 3, 5, 10, 15, 20, 25, 30, 45, 60, 75, or 90 days of being administered the composition. In some examples, the method includes selecting a subject who has not received prednisone within the past 1, 3, 5, 10, 15, 20, 25, 30, 45, 60, 75, or 90 days. In some examples, the subject received the organ transplant 0.5-1, 1-2, 3-4, 1-5, 1-10, 3-10, 3-6, 6-10, 10-20, or 20-40 years prior to receiving the composition. In some examples, the method includes selecting a subject who received an organ transplant within the prior 0.5-1, 1-2, 3-4, 1-5, 1-10, 3-10, 3-6, 6-10, 10-20, or 20-40 years. In some examples, the subject was receiving tacrolimus, sirolimus, mycophenolate mofetil, mycophenolate sodium, and/or etanercept prior to receiving the composition. In some examples, the method includes selecting a subject receiving tacrolimus, sirolimus, mycophenolate mofetil, mycophenolate sodium, and/or etanercept.

In some examples, the method includes administering one or more immunosuppressive agents to the subject that are the same as one or more of the immunosuppressive agents administered to the subject prior to receiving the composition. In some examples, the method includes administering the one or more immunosuppressive agents to the subject at the same concentration as the same one or more immunosuppressive agents administered to the subject prior to receiving the composition. In some examples, the method includes administering the one or more immunosuppressive agents to the subject at the same frequency (e.g., daily, every other day, twice weekly, or monthly) as the same one or more immunosuppressive agents administered to the subject prior to receiving the composition. In some embodiments, the one or more immunosuppressive agents were administered to the subject prior to receiving the composition because the subject had previously received an organ transplant (e.g., a kidney or liver transplant). In some embodiments, the one or more immunosuppressive agents were administered to the subject prior to receiving the composition to prevent the subject's immune system from rejecting a previously received organ (e.g., a kidney transplant or a liver transplant).

In some examples, the transplanted organ is from a human. In some examples, the transplanted organ is not an in vitro derived (e.g., in vitro stem cell-derived) organ.

In some examples, at least 50%, 60%, 70%, 80%, 90%, 93%, 95%, 96%, 97%, 98%, 99% or 100% of the clusters in the composition used in the treatment are less than 200 μm in diameter. In some examples, 50-100%, 70-100%, 80-100%, 90-100%, 95-100%, 90-95%, 95-97%, or 97-99%, of the clusters in the composition used in the treatment are less than 200 μm in diameter. In some examples, at least 90%, 93%, 95%, 96%, 97%, 98%, 99% or 100% of the clusters in the composition used in the treatment are less than 200 μm in diameter. In some examples, 90-100%, 95-100%, 90-95%, 95-97%, or 97-99%, of the clusters in the composition used in the treatment are less than 200 μm in diameter. In some examples, at least 50%, 60%, 70%, 80%, 90%, 93%, 95%, 96%, 97%, 98%, 99% or 100% of the clusters in the composition used in the treatment are less than 200 μm in diameter. In some examples, 50-100%, 70-100%, 80-100%, 90-100%, 95-100%, 90-95%, 95-97%, or 97-99% of the cells in the composition used in the treatment are in one or more cell clusters. In some examples, at least 90%, 93%, 95%, 96%, 97%, 98%, 99% or 100% of the clusters in the composition used in the treatment are less than 200 μm in diameter. In some examples, 90-100%, 95-100%, 90-95%, 95-97%, or 97-99% of the cells in the composition used in the treatment are in one or more cell clusters.

In some examples, the subject did not receive a cadaveric islet transplant prior to receiving the composition. In some examples, the method includes selecting a subject who did not receive a cadaveric islet transplant. In some examples, the transplanted organ is not from a non-human animal (e.g., not from a pig). In some examples, the method does not include administering tacrolimus, sirolimus, and/or mycophenolate mofetil to the subject. In some examples, the subject has Type 1 Diabetes. In some examples, the method includes selecting a subject who has Type 1 Diabetes.

### Methods of Treatment

In some aspects, provided herein are methods for treating a disease characterized by high blood sugar levels over a prolonged period of time, e.g., diabetes, e.g., Type 1 diabetes in a subject. In some aspects, the subject previously received an organ transplant, and/or is selected due to having received an organ transplant. In some aspects, provided herein are methods for preventing a disease characterized by high blood sugar levels over a prolonged period of time, e.g., diabetes, e.g., Type 1 diabetes in a subject, e.g., a subject at risk of developing such a disease. In some aspects, provided herein are methods for reducing likelihood of developing a disease characterized by high blood sugar levels over a prolonged period of time, e.g., diabetes, e.g., Type 1 diabetes in a subject. In some embodiments, the disclosure provides for immunosuppression.

In some embodiments, the disclosure provides for a method in which a subject is administered any of the cell compositions disclosed herein (e.g., a composition comprising any of the cell populations disclosed herein) at least 3, 4, 5, 6, 7, 8, 10, 12, 14, 17, 20, 23, 28, 31, 33 or 35 days after the subject is administered an immunosuppression-induction agent (e.g., any of the immunosuppression-induction agents disclosed herein). In some embodiments, the disclosure provides for a method in which a subject is administered any of the cell compositions disclosed herein (e.g., a composition comprising any of the cell populations disclosed herein) between 3-28, 3-21, 3-14, 3-7, 3-5, 4-6, 5-18, 5-8, 7-10, 7-14, 10-14, 10-21, 10-28, 10-45, 14-21, 14-28, 14-35, 21-28, 21-35, or 28-35 days after the subject is administered an immunosuppression-induction agent (e.g., any of the immunosuppression-induction agents disclosed herein).

In some embodiments, the disclosure provides for a method of treating a subject having diabetes, comprising: a) treating the subject with one or more immunosuppression-induction agents, and b) treating the subject with a composition comprising a population of cells, wherein the population comprises a plurality of cells that express ISL1, and wherein step b) is performed between 3 and 35 days after step a). In some embodiments, the subject is treated with a single type of immunosuppression-induction agent (e.g. ATG-R). In some embodiments, the subject is treated with multiple different immunosuppression-induction agents. In some embodiments, step b) is performed 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days after step a). In some embodiments, step b) is performed between 3-28, 3-21, 3-14, 3-7, 3-5, 5-18, 5-8, 7-10, 7-14, 10-14, 10-21, 10-28, 10-45, 14-21, 14-28, 14-35, 21-28, 21-35, or 28-35 days after step a). In some embodiments, step b) is performed between 6-28 or 10-21 days after step a). In some embodiments, the immunosuppression-induction agent is an anti-thymocyte globulin binding moiety (e.g., ATG-R, ATG-F, ATGAM). In some embodiments, the immunosuppression-induction agent is an anti-CD3 binding moiety (e.g., OKT3). In some embodiments, the immunosuppression-induction agent is an anti-CD25 binding moiety (e.g., daclizumab or basiliximab). In some embodiments, the immunosuppression-induction agent is an anti-CD52 binding moiety (e.g., alemtuzumab). In some embodiments, the immunosuppression-induction agent is a high dose of an immunosuppression maintenance agent (e.g., bolus glucocorticosteroids or intravenous cyclosporine [CsA]). In some embodiments, the immunosuppression induction agent is methylprednisolone at a dose greater than 9 mg/kg (e.g., 9-30, 9-25, 9-20, 9-15, 15-30, 15-25, 15-20, 20-30 or 20-25 mg/kg). In some embodiments, the immunosuppression-induction agent is an anti-thymocyte globulin binding moiety.

In some embodiments, step a) comprises administering multiple doses of the immunosuppression-induction agent. In some embodiments, step a) comprises administering 1, 2, 3, 4, 5, 6, 7, 8, or 9 doses of the immunosuppression-induction agent. In some embodiments, step a) comprises administering 1, 2 or 3 doses of the immunosuppression-induction agent. In some embodiments, step a) comprises administering multiple doses of the immunosuppression-induction agent in a single day. In some embodiments, a second dose of the immunosuppression-induction agent is administered to a subject within 3-24, 3-18, 3-12, 3-9, 3-6, or 4-6 hours of a first dose of an immunosuppression-induction agent. In some embodiments, a third dose of the immunosuppression-induction agent is administered to a subject within 3-24, 3-18, 3-12, 3-9, 3-6, or 4-6 hours of a second dose of an immunosuppression-induction agent.

In some embodiments, the method comprises administering multiple doses of the immunosuppression-induction agent, wherein the second dose is higher than the first dose. In some embodiments, the method comprises administering multiple doses of the immunosuppression-induction agent, wherein the third dose is higher than the first and second doses. In some embodiments, the method comprises administering a first dose of the immunosuppression-induction agent (e.g., an anti-thymocyte globulin binding moiety) at 0.1-1.25, 0.2-0.8, 0.2-0.7, 0.2-0.6, 0.3-0.6, 0.4-0.6, or 0.45-0.55 mg/kg (e.g., 0.25 or 0.5 mg/kg). In some embodiments, the method comprises administering a second dose of the immunosuppression-induction agent (e.g., an anti-thymocyte globulin binding moiety) at 0.25-2, 0.25-1.5, 0.25-1.25, 0.5-1.5, 0.5-1.25, 0.75-1.25, 0.9-1.1 (e.g., 0.5 or 1.0 mg/kg). In some embodiments, the method comprises administering an additional dose (e.g., a third, fourth, fifth or sixth) of the immunosuppression-induction agent (e.g., an anti-thymocyte globulin binding moiety) at 0.25-3, 0.5-2.5, 0.5-2, 1-2, 1.25-1.75, 0.25-1, or 0.5-0.8 (e.g., 1.0 or 1.5 mg/kg).

In some embodiments, the subject is administered (e.g., intravenously) an anti-CD25 binding moiety (e.g., basiliximab). In some embodiments, if the subject is administered a second dose of a population of cells, the subject is administered (e.g., intravenously) an anti-CD25 binding moiety (e.g., basiliximab). In some embodiments, the method comprises administering (e.g., intravenously) an anti-CD25 binding moiety (e.g., basiliximab) at a dose of 1-50, 1-40, 1-30, 1-20, 1-10, 10-50, 10-40, 10-30, 10-20, 15-20, 18-22, or 19-21 mg.

In some embodiments, step a) comprises administering multiple doses of the immunosuppression-induction agent over several days. In some embodiments, step a) comprises administering 1, 2, or 3 doses of the immunosuppression-induction agent on a first day, followed by administering 1, 2 or 3 doses of the immunosuppression-induction agent on a second day. In some embodiments, step a) comprises administering one or more doses (e.g., 1, 2, or 3 doses) of the immunosuppression-induction agent on a first day, and then administering one or more doses (e.g., 1, 2 or 3 doses) on a second day, wherein the second day is 1-35, 1-28, 1-21, 1-14, 1-7, 1-5, 1-3, 1-2, or 1 day after the administration of the doses of the immunosuppression-induction agent on the first day. In some embodiments, step a) comprises administering one or more doses (e.g., 1, 2, or 3 doses) of the immunosuppression-induction agent on a first day, and then administering one or more doses (e.g., 1, 2 or 3 doses) on a second day, wherein the second day is 1-35, 1-28, 1-21, 1-14, 1-7, 1-5, 1-3, 1-2, or 1 day after the administration of the doses of the immunosuppression-induction agent on the first day, and then administering one or more doses (e.g., 1, 2, or 3 doses) on a third day, wherein the third day is 1-35, 1-28, 1-21, 1-14, 1-7, 1-5, 1-3, 1-2, or 1 day after the administration of the doses of the immunosuppression-induction agent on the second day. In some embodiments, step a) comprises administering one or more doses (e.g., 1, 2, or 3 doses) of the immunosuppression-induction agent on a first day, and then administering one or more doses (e.g., 1, 2 or 3 doses) on a second day, wherein the second day is 1-35, 1-28, 1-21, 1-14, 1-7, 1-5, 1-3, 1-2, or 1 day after the administration of the doses of the immunosuppression-induction agent on the first day, and then administering one or more doses (e.g., 1, 2, or 3 doses) on a third day, wherein the third day is 1-35, 1-28, 1-21, 1-14, 1-7, 1-5, 1-3, 1-2, or 1 day after the administration of the doses of the immunosuppression-induction agent on the second day, and then administering one or more doses (e.g., 1, 2, or 3 doses) on a fourth day, wherein the third day is 1-35, 1-28, 1-21, 1-14, 1-7, 1-5, 1-3, 1-2, or 1 day after the administration of the doses of the immunosuppression-induction agent on the third day.

In some embodiments, step a) comprises administering 1, 2, or 3 doses of the immunosuppression-induction agent, and wherein the method further comprises the step of administering an immunosuppression-induction agent within 3 days prior to step b). In some embodiments, step a) comprises administering 1, 2, or 3 doses of the immunosuppression-induction agent, and wherein the method further comprises the step of administering 1, 2 or 3 doses of an immunosuppression-induction agent within 3 days prior to step b). In some embodiments, step a) comprises administering 1, 2, or 3 doses of the immunosuppression-induction agent, and wherein the method also comprises the step of administering 1, 2 or 3 doses of an immunosuppression-induction agent within 1, 2, or 3 days after step b).

In some embodiments, a dose of the immunosuppression-induction agent (e.g., an anti-thymocyte globulin binding moiety) is administered at 0.05-3 mg/kg. In some embodiments, the total immunosuppression-induction agent (e.g., an anti-thymocyte globulin binding moiety) administered in the method is 1 mg/kg to 10 mg/kg; 2 mg/kg to 9 mg/kg; 3 mg/kg to 8 mg/kg; 4 mg/kg to 7 mg/kg; or 5 mg/kg to 6 mg/kg. In some embodiments, the composition comprises about 1×10⁸ to about 10×10⁸ cells.

In some embodiments, the method further comprises treating the subject with a Tumor Necrosis Factor-alpha (TNFα) inhibitor. In some embodiments, the subject is treated with the TNFα during step a) and/or b) or within between 1 minute and 2 days, 1 minute and 1 hour, 1 hour and 6 hours, 6 hours and 24 hours, 1 day and 2 days, or 2-4 days of step a) and/or b). In some embodiments, the subject is treated with 10-100 mg of a Tumor Necrosis Factor-alpha (TNFα) inhibitor.

In some embodiments, step a) and/or b) further comprises treating the subject with sirolimus and/or tacrolimus. In some embodiments, the subject is treated with sirolimus and/or tacrolimus during step a) and/or b) or within between 1 minute and 2 days, 1 minute and 1 hour, 1 hour and 6 hours, 6 hours and 24 hours, 1 day and 2 days, or 2-4 days of a) and/or step b). In some embodiments, the method comprises treating the subject with 0.01-5000 mg/kg of sirolimus. In some embodiments, the method comprises treating the subject with 0.001-0.05 mg/kg of tacrolimus.

In some embodiments, the method comprises treating the subject with mycophenolate mofetil or mycophenolate sodium. In some embodiments, the method comprises treating the subject with 100-5000 mg of mycophenolate mofetil or mycophenolate sodium. In some embodiments, the subject is treated with mycophenolate mofetil or mycophenolate sodium during step a) and/or b) or within between 1 minute and 2 days, 1 minute and 1 hour, 1 hour and 6 hours, 6 hours and 24 hours, 1 day and 2 days, or 2-4 days of step a) and/or b).

In some embodiments, the subject is treated with an antimicrobial agent. In some embodiments, the method further comprises c) administering to the subject a second composition comprising a second population of cells, and d) administering to the subject an anti-CD25 binding moiety (e.g., basiliximab) concurrently with or after the administration of the second composition; wherein the second composition comprises a plurality of cells that express ISL1. In some embodiments, the method comprises administering (e.g., intravenously) the anti-CD25 binding moiety (e.g., basiliximab) at a dose of 1-50, 1-40, 1-30, 1-20, 1-10, 10-50, 10-40, 10-30, 10-20, 15-20, 18-22, or 19-21 mg.

In some embodiments, the method further comprises c) administering to the subject a second composition comprising a second population of cells, and d) administering to the subject a vasoactive agent (e.g., pentoxifylline) concurrently with or after the administration of the second composition; wherein the second composition comprises a plurality of cells that express ISL1. In some embodiments, the population of cells comprises a plurality of NKX6.1-positive and ISL1-positive cells.

### Organ Transplant Recipients

In some embodiments, the disclosure provides for methods of treating a subject, who previously received an organ transplant, such as a subject who has diabetes. In some embodiments the disclosure provides for selecting a subject with diabetes for transplant, such as a selecting a subject who previously received an organ transplant.

As used herein “organ transplant” refers to a procedure in which a tissue or an organ is obtained from a donor source and introduced into a recipient subject (e.g., a subject to be treated using the methods provided herein). Organ transplantation is typically intended to replace a recipient's damaged or failing organ or tissue with a functional counterpart. An organ transplant can be an autograft, an allograft, an isograft, or a xenograft. In one aspect, an organ transplant is an autograft. In one aspect, an organ transplant is an allograft. As used herein an “allograft organ transplant” is a transplant between genetically non-identical members of the same species. In some embodiments, the donor source is a living donor. In some embodiments, the donor source is cadaveric. In some examples the donor source has a human genetic background. In some examples, the donor source is a human. In some embodiments, the donor source is non-human, such as a pig. In some embodiments, the donor source is in vitro derived. In some embodiments, the donor source is not an in vitro derived (e.g., in vitro stem cell-derived) organ. In some embodiments, the recipient subject received, or is receiving one or more immunosuppressive agents to prevent, mitigate, or reduce the incidence of organ transplant rejection.

In some embodiments, the organ transplant is a corneal transplant, a bone transplant, a bone marrow transplant, an eye transplant, a hand transplant, a heart transplant, a heart-lung transplant, a heart valve transplant, a hematopoietic stem cell transplant, an intestine transplant, an islet transplant, a kidney transplant, a large intestine transplant, a liver transplant, a lung transplant, a multivisceral transplant, an ovarian transplant, a pancreas transplant, a penile transplant, a skin transplant, a small intestine transplant, a stomach transplant, a testis transplant, a thymic transplant, or a uterine transplant. In particular embodiments, the organ transplant is a kidney transplant. In further particular embodiments, the organ transplant is a liver transplant. In some embodiments, the organ transplant is a solid organ transplant. In some embodiments, the organ transplant is not a corneal transplant, not a bone marrow transplant, and/or not a thymic transplant. In some embodiments, the organ transplant is not an islet transplant and/or not a pancreatic transplant.

In some embodiments, the disclosure provides for methods of treating a subject who has diabetes, wherein the subject previously received an organ transplant. In some embodiments, the method includes selecting a subject who previously received a corneal transplant, a bone transplant, a bone marrow transplant, an eye transplant, a hand transplant, a heart transplant, a heart-lung transplant, a heart valve transplant, a hematopoietic stem cell transplant, an intestine transplant, an islet transplant, a kidney transplant, a large intestine transplant, a liver transplant, a lung transplant, a multivisceral transplant, an ovarian transplant, a pancreas transplant, a penile transplant, a skin transplant, a small intestine transplant, a stomach transplant, a testis transplant, a thymic transplant, and/or a uterine transplant. In some embodiments, the method includes selecting a subject who did not previously receive a corneal transplant, not a bone marrow transplant, and/or not a thymic transplant. In some embodiments, the method includes selecting a subject who did not previously receive an islet transplant and/or did not previously receive a pancreatic transplant. In selected embodiments, the method includes selecting a subject who previously received a kidney transplant. In further selected embodiments, the method includes selecting a subject who previously received a liver transplant

In some embodiments, the subject received the organ transplant about 0.5-1, about 1-2, about 3-4, about 1-5, about 1-10, about 3-10, about 3-6, about 6-10, about 10-20, or about 20-40 years prior to treatment with the disclosed methods. In some embodiments, the method includes selecting a subject who received the organ transplant about 0.5-1, about 1-2, about 3-4, about 1-5, about 1-10, about 3-10, about 3-6, about 6-10, about 10-20, or about 20-40 years prior to treatment with the disclosed methods.

### Immunosuppressive Agents

In some embodiments, the disclosure provides for methods of treating a subject with one or more immunosuppressive agents, such as the subject having diabetes who previously received an organ transplant. In some embodiments the disclosure provides for selecting a subject receiving treatment with one or more immunosuppressive agents for treatment, such as selecting a subject who also has diabetes and/or who previously received an organ transplant. In some embodiments, prior to administering to the subject any of the compositions disclosed herein comprising any of the stem cell-derived islet cells disclosed herein, the subject already received one or more immunosuppressive agents, e.g., in association with the organ transplant. In some embodiments, the subject received any of immunosuppressive agents disclosed herein within 1 week, 2 weeks, 3 weeks, 4 weeks, 2 months, 3 months or 6 months of being administered the composition comprising the stem cell-derived islet cells. The immunosuppressive agents used in the disclosed methods can include immunosuppression-induction agents and/or immunosuppression-maintenance agents. In some embodiments, the immunosuppressive agents are not immunosuppression-induction agents. In some embodiments, the immunosuppressive agents are not immunosuppression-maintenance agents.

Examples of immunosuppressive agents include an anti-thymocyte globulin binding moiety, ATG-R, ATG-F, ATGAM, an anti-CD3 binding moiety, OKT3, an anti-CD25 binding moiety, daclizumab, basiliximab, an anti-CD52 binding moiety, alemtuzumab, a calcineurin inhibitor, tacrolimus, cyclosporine, an antiproliferative agent, mycophenolate mofetil, mycophenolate sodium, azathioprine, an mTOR inhibitors, sirolimus, everolimus, steroids, glucocorticosteroids, prednisone, hydrocortisone, deflazacort, prednisolone, methylprednisolone, triamcinolone, dexamethasone, intravenous cyclosporine [CsA], a TNFα, inhibitor, adalimumab, infliximab, certolizumab pegol, golimumab, belatacept and/or zotarolimus.

Immunosuppressive agents can be administered via any effective route. The immunosuppressive agents disclosed herein can be administered in sufficient amounts to modulate or reduce a transplant rejection response against an organ transplant, such as a solid organ transplant, such a kidney transplant, or such as a liver transplant. Administration can be effected in one dose, continuously or intermittently throughout the course of a treatment.

Any administration route, systemic or local, can be used to deliver the immunosuppressive agent(s). In some examples, the immunosuppressive agent(s) may be parenterally administered by injection, infusion or implantation. In some examples, the immunosuppressive agent(s) may be administered intravenously, intraventricularly, intra-aurally, intra-ocularly, or peri-ocularly, orally, parenterally, subcutaneously, intramuscularly, intrapleurally, topically, intralymphatically, intra-arterially, intracardiac, subventricular, sub-retinally, intravitreally, intraarticular, sublingually, rectally, transdermally, intranasally, intraductally, sublingually, vaginally, via inhalation, or any combination thereof. In some examples, the immunosuppressive agent is administered under the direction of a supervising clinician and/or is administered by a clinician. In some embodiments, the immunosuppressive agent(s) are administered locally to a transplanted organ, such as the kidney or such as the liver. In some embodiments, the immunosuppressive agent(s) are administered systemically.

Pharmaceutical forms suitable for administration include sterile aqueous solutions or dispersions, and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations may contain a preservative to prevent the growth of microorganisms. In many cases the form is sterile and fluid to the extent that easy syringeability exists. The forms may also be stable under the conditions of manufacture and storage, and may be preserved against the contaminating action of microorganisms, such as bacteria and fungi.

The carrier may be a solvent or dispersion medium including, for example, water, ethanol, polyol (e.g., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms may be effected by various agents, for example, parabens, chlorobutanol, phenol, sorbic acid, thimerosal, and the like. In some examples, it will be preferable to include isotonic agents, such as sugars or sodium chloride. If prolonged absorption of the compositions is desired, agents delaying absorption, for example, aluminum monostearate and gelatin, may be included in the compositions.

For administration of an aqueous solution, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host. Sterile solutions for parenteral administration may be prepared by including the immunosuppressive agent(s) at an effective amount in the appropriate solvent with various other ingredients, as required or desired, followed by filtered sterilization. In aspects, a pharmaceutical composition includes an effective amount of the immunosuppressive agent(s), as disclosed herein, and a pharmaceutically acceptable carrier. In some aspects, the pharmaceutical composition further includes a population of stem cell-derived islet cells.

Pharmaceutical compositions can be produced that contain one or more immunosuppressive agents, a pharmaceutically acceptable excipient, and optionally, a population of stem cell-derived islet cells. Such excipients can include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences, 23rd Edition, Academic Press, Elsevier, (2020).

In some embodiments, the one or more immunosuppressive agents include an anti-thymocyte globulin binding moieties (e.g., ATG-R, ATG-F, or ATGAM), an anti-CD3 binding moiety (e.g., OKT3), an anti-CD25 binding moiety (e.g., daclizumab or basiliximab), an anti-CD52 binding moiety (e.g., alemtuzumab), or a high dose of an immunosuppression maintenance agent (e.g., bolus glucocorticosteroids or intravenous cyclosporine [CsA]). In particular embodiments, the immunosuppressive agent is an anti-thymocyte globulin binding moiety. In some embodiments, the method comprises administering multiple doses of the immunosuppressive agent. In some embodiments, the method comprises administering multiple doses of the immunosuppressive agent in a single day. In some embodiments, the method comprises administering multiple doses of an immunosuppression-induction agent over several days. In some embodiments, a dose of the immunosuppressive agent is administered at about 0.05-3 mg/kg.

In some embodiments, the method comprises treating the subject with a Tumor Necrosis Factor-alpha (TNFα) inhibitor. In some embodiments, the subject is treated with about 10-100 mg of a Tumor Necrosis Factor-alpha (TNFα) inhibitor. In some embodiments, the method comprises treating the subject with sirolimus, (e.g., about 0.01-5000 mg/kg of sirolimus). In some embodiments, the method comprises treating the subject with tacrolimus (e.g., about 0.001-0.05 mg/kg of tacrolimus). In some embodiments, the method comprises treating the subject with mycophenolate mofetil and/or mycophenolate sodium. In some embodiments, the method comprises treating the subject with about 100-5000 mg of mycophenolate mofetil and/or with about 100-5000 mg mycophenolate sodium.

In some embodiments, the subject is treated with about 0.01-1, about 0.02-0.3, about 0.02-about 0.15, about 0.02-0.12, about 0.02-0.08, about 0.03-0.07, about 0.04-0.15, or about 0.05-2 mg/kg sirolimus. In some embodiments, the sirolimus is administered daily. In some embodiments, the sirolimus is administered orally. In some embodiments, the subject is treated with about 0.001-0.05, about 0.003-0.03, about 0.004-0.02, about 0.006-0.02, about 0.008-0.02, about 0.01-0.02, about 0.009-0.03, and/or about 0.009-0.02 mg/kg tacrolimus. In some embodiments, the tacrolimus is administered daily. In some embodiments, the tacrolimus is administered orally. In some embodiments, the first dose of sirolimus is about 0.05 to 0.2 mg/kg and subsequent doses are about 0.05 to 0.1 mg/kg once each morning. In some embodiments, the sirolimus dose is adjusted to maintain a whole blood 24-hour trough target of about 10 to 15 ng/ml for the first 3 months after administration of the composition. In some embodiments, the sirolimus dose is adjusted to maintain a whole blood 24-hour trough target of about 8 to 12 ng/ml after the first 3 months after administration of the composition. In some embodiments the administration of tacrolimus is started after the day of the administration of the composition comprising the population of cells (Day 1). In some embodiments, the administration of tacrolimus is started about between Day 2 and Day 5 of the administration of the composition. In some embodiments, the administration of tacrolimus is started on about Day 2 of the administration of the first pharmaceutical composition. In some embodiments, the tacrolimus is started at a dose of about 0.015 mg/kg given every 12 hours. In some embodiments, the tacrolimus dose is adjusted to maintain a whole blood 12-hour trough of about 3 to 6 ng/ml. In some embodiments, if the subject develops intolerable or clinically undesirable side-effects related to sirolimus and/or tacrolimus treatment, the sirolimus and/or tacrolimus is replaced with mycophenolate mofetil and/or mycophenolate sodium. In some embodiments, mycophenolate mofetil is dosed between about 500 to 1000 mg twice a day. In some embodiments, mycophenolate sodium is dosed between about 360 to 720 mg twice a day. In some embodiments, the subject is treated with about 100-5000, about 100-3000, about 100-2000, about 100-1000, about 300-5000, about 300-2000, about 300-1000, about 500-1000, about 700-1200, about 800-1200, about 400-900, about 400-700 mg of mycophenolate mofetil and/or mycophenolate sodium. In some embodiments, the mycophenolate mofetil and/or mycophenolate sodium is administered daily. In some embodiments, the mycophenolate mofetil and/or mycophenolate sodium is administered orally. In some embodiments, the subject received multiple doses of immunosuppressive agents prior to being administered the composition.

In some embodiments, the subject received one or more immunosuppressive agents for at least about 1 month, about 2 months, about 3 months, about 6 months, about 9 months, about 1 year, about 2 years, about 3 years, about 4 years, about 5 years, or about 6 years prior to being administered the composition. In some embodiments, the subject received immunosuppressive agents for about 1-12 months, about 1-9 months, about 1-6 months, about 1-3 months, about 1-2 months, about 1-10 years, about 10-20 years, about 20-40 years, about 1-7 years, about 1-5 years, about 1-3 years, about 1-2 years, or about 1-1.5 years prior to being administered the composition. In some embodiments, the subject received immunosuppressive agents on at least an about daily, about twice daily, about twice weekly, about three times a week, about weekly, about bi-weekly or about monthly basis prior to being administered the composition. In some embodiments, the subject is not administered a steroid within about 1, about 3, about 5, about 10, about 15, about 20, about 25, about 30, about 45, about 60, about 75, or about 90 days of being administered the composition. In some embodiments, the subject is not administered prednisone within about 1, about 3, about 5, about 10, about 15, about 20, about 25, about 30, about 45, about 60, about 75, or about 90 days of being administered the composition. In some embodiments, the subject was receiving tacrolimus, sirolimus, mycophenolate mofetil, mycophenolate sodium, and/or etanercept prior to receiving the composition.

In some embodiments, the method includes selecting a subject who received one or more immunosuppressive agents for at least about 1 month, about 2 months, about 3 months, about 6 months, about 9 months, about 1 year, about 2 years, about 3 years, about 4 years, about 5 years, or about 6 years prior to being administered the composition. In some embodiments, the method includes selecting a subject who received immunosuppressive agents for about 1-12 months, about 1-9 months, about 1-6 months, about 1-3 months, about 1-2 months, about 1-10 years, about 10-20 years, about 20-40 years, about 1-7 years, about 1-5 years, about 1-3 years, about 1-2 years, or about 1-1.5 years prior to being administered the composition. In some embodiments, the method includes selecting a subject who received immunosuppressive agents on at least an about daily, about twice daily, about twice weekly, about three times a week, about weekly, about bi-weekly or about monthly basis prior to being administered the composition. In some embodiments, the method includes selecting a subject who was not administered a steroid within about 1, about 3, about 5, about 10, about 15, about 20, about 25, about 30, about 45, about 60, about 75, or about 90 days of being administered the composition. In some embodiments, the method includes selecting a subject who was not administered prednisone within about 1, about 3, about 5, about 10, about 15, about 20, about 25, about 30, about 45, about 60, about 75, or about 90 days of being administered the composition. In some embodiments, the method includes selecting a subject who was receiving tacrolimus, sirolimus, mycophenolate mofetil, mycophenolate sodium, and/or etanercept prior to receiving the composition.

In some embodiments, the method includes administering one or more immunosuppressive agents to the subject that are the same as one or more of the immunosuppressive agents administered to the subject prior to receiving the composition. In some embodiments, the method includes administering the one or more immunosuppressive agents to the subject at the same concentration as the same one or more immunosuppressive agents administered to the subject prior to receiving the composition. In some embodiments, the method includes administering the one or more immunosuppressive agents to the subject at the same frequency (e.g., about daily, about every other day, about twice weekly, or about monthly) as the same one or more immunosuppressive agents administered to the subject prior to receiving the composition. In some embodiments, the method does not comprise a step of administering tacrolimus, sirolimus, and/or mycophenolate mofetil to the subject.

In some embodiments, the subject is administered an anti-thymocyte globulin binding moiety. In some embodiments, the subject is administered a first dose of about 0.05-3 mg/kg anti-thymocyte globulin binding moiety within about 72-24 hours prior to being treated with the composition. In some embodiments, the anti-thymocyte globulin moiety is administered via infusion. In some embodiments, the infusion is via a high-flow vein. In some embodiments, the infusion lasts for about 2-20, about 3-18, about 4-15, about 5-13, about 6-12, about 7-11, about 8-10, or about 9-11 hours. In some embodiments, the subject is not administered any anti-thymocyte globulin binding moiety for a resting period between doses. In some embodiments, the resting period is about 1-20, about 6-15, about 12-24, about 10-20, about 1-12, about 2-11, about 3-10, about 4-9, about 5-8, about 5-7, about 5-15, about 5-20, about 8-10, or about 9-14 hours. In particular embodiments, the first dose of the anti-thymocyte globulin binding moiety administration protocol is infused over a period of about 6-12 hours. In particular embodiments, a subsequent dose of the anti-thymocyte globulin binding moiety administration protocol is infused over a period of about 6-12 hours. In some embodiments, there are at least about 6 hours between the end of one anti-thymocyte globulin binding moiety infusion and the start of a subsequent anti-thymocyte globulin binding moiety infusion.

In some embodiments, the total dose of anti-thymocyte globulin binding moiety at the end of the anti-thymocyte globulin binding moiety administration protocol is between about 1 mg/kg to 10 mg/kg, about 2 mg/kg to 9 mg/kg, about 3 mg/kg to 8 mg/kg, about 4 mg/kg to 7 mg/kg, or about 5 mg/kg to 6 mg/kg. In particular embodiments, the total dose of the anti-thymocyte globulin binding moiety administration is about 6 mg/kg. In some embodiments, the anti-thymocyte globulin binding moiety administration protocol includes dosing the first dose of anti-thymocyte globulin binding moiety administration at a lower dose than any subsequent dose of anti-thymocyte globulin binding moiety. In some embodiments, the anti-thymocyte globulin binding moiety administration protocol includes monitoring of total white blood cell (WBC) and/or platelet counts of the subject following a dose of the anti-thymocyte globulin binding moiety; and reducing a subsequent dose of anti-thymocyte globulin binding moiety (e.g., by at least about 10%, about 20%, about 30%, about 40%, about 50%, about 60%, about 70%, about 80%, about 90% or about 100%) if the WBC count is between about 2,000 and 3,000 cells/mm³. In some embodiments, the anti-thymocyte globulin binding moiety administration protocol includes monitoring of total white blood cell (WBC) and/or platelet counts of the subject following a dose of the anti-thymocyte globulin binding moiety; and reducing a subsequent dose of anti-thymocyte globulin binding moiety (e.g., by at least about 10%, about 20%, about 30%, about 40%, about 50%, about 60%, about 70%, about 80%, about 90% or about 100%) if the platelet count is between about 50,000 and 75,000 cells/mm³. In some embodiments, the anti-thymocyte globulin binding moiety administration protocol includes monitoring of total white blood cell (WBC) and/or platelet counts of the subject following a dose of the anti-thymocyte globulin binding moiety; and reducing a subsequent dose of anti-thymocyte globulin binding moiety (e.g., by at least about 10%, about 20%, about 30%, about 40%, about 50%, about 60%, about 70%, about 80%, about 90% or about 100%) if the WBC count is between about 2,000 and 3,000 cells/mm³ and if the platelet count is between about 50,000 and 75,000 cells/mm³. In some embodiments, the anti-thymocyte globulin binding moiety administration protocol includes monitoring of total white blood cell (WBC) and/or platelet counts of the subject following a dose of the anti-thymocyte globulin binding moiety and stopping the anti-thymocyte globulin binding moiety administration if the WBC count falls below about 2,000 cells/mm³. In some embodiments, the anti-thymocyte globulin binding moiety administration protocol includes monitoring of total white blood cell (WBC) and/or platelet counts of the subject following a dose of the anti-thymocyte globulin binding moiety and stopping the anti-thymocyte globulin binding moiety administration if the platelet count falls below about 50,000 cells/mm³. In some embodiments, the anti-thymocyte globulin binding moiety administration protocol includes monitoring of total white blood cell (WBC) and/or platelet counts of the subject following a dose of the anti-thymocyte globulin binding moiety and stopping the anti-thymocyte globulin binding moiety administration if the WBC count falls below about 2,000 cells/mm³ and if the platelet count falls below about 50,000 cells/mm³.

In some embodiments, the method comprises administering to the subject at least one immune response modulator before or concurrently with the administration of the second pharmaceutical composition. In some embodiments, the at least one immune response modulator and/or the second pharmaceutical composition form part of a system or kit. In some embodiments, the at least one immune response modulator administered before or concurrently with the administration of the second pharmaceutical composition comprises THYMOGLOBULIN®, Etanercept, Basiliximab, Tacrolimus, Sirolimus, and/or Mycophenolate mofetil. In particular embodiments, the subject is administered Basiliximab. In some embodiments, the method comprises the step of administering an anti-CD25 binding moiety (e.g., basiliximab) concurrently with or after the administration of the second composition, optionally wherein the first and the second composition each comprise a population of cells that express NKX6.1 and ISL1. In particular embodiments, the subject is administered multiple doses of basiliximab. In some embodiments, the subject is administered basiliximab on the day the second composition is administered to the subject. In some embodiments, a first administration of basiliximab administration occurs on the day of administration of the second pharmaceutical (Day A1) followed by a second administration of basiliximab between about Day A2 and Day A8 of the of the administration of the second composition. In some embodiments, a first administration of Basiliximab administration occurs on the day of administration of the second pharmaceutical (Day A1) followed by a second administration of basiliximab between about Day A3 and Day A7 of the of the administration of the second composition. In some embodiments, a first administration of basiliximab administration occurs on the day of administration of the second pharmaceutical (Day A1) followed by a second administration of basiliximab between about Day A4 and Day A6 of the of the administration of the second composition. In some embodiments, the second administration of basiliximab occurs on about Day A5 of the of the administration of the second pharmaceutical. In some embodiments, the basiliximab is administered at a dose of about 5 mg to 50 mg, about 10 mg to 45 mg, about 15 mg to 40 mg, and/or about 20 mg to 30 mg. In particular embodiments, the basiliximab is administered at a dose of about 20 mg. In some embodiments, the basiliximab is administered intravenously.

In some embodiments, the subject is administered a TNFα inhibitor. In some embodiments, the subject is administered 2, 3, 4, 5, 6, or 7 doses of the TNFα inhibitor in the month following the administration of the composition comprising the population of cells. In some embodiments, the subject is treated with the TNFα inhibitor about 3 days, about 4 days, about 5 days, about 6 days, about 7 days, about 8 days, about 9 days, about 10 days, about 11 days, about 12 days, about 13 days, about 14 days, and/or about 15 days following the administration of the composition. In some embodiments, the subject is administered about 5-500, about 5-100, about 5-75, about 5-50, about 10-100, about 10-75, about 10-50, about 30-100, or about 30-75 mg TNFα inhibitor for each dose of the TNFα inhibitor. In some embodiments, the TNFα inhibitor is administered subcutaneously and/or intravenously. In some embodiments, the TNFα inhibitor includes etanercept, adalimumab, infliximab, certolizumab pegol, and/or golimumab.

In particular embodiments, the subject is administered etanercept. In some embodiments, etanercept is administered on the same day as the administration of the composition comprising the population of cells. In some embodiments, etanercept is administered on the same day as the administration of the composition comprising the population of cells (Day 1) followed by further administration on Day 2, Day 3, Day 4, Day 5, Day 6, Day 7, Day 8, Day 9, Day 10, Day 11, Day 12, Day 13, Day 14, and/or Day 15. In some embodiments, etanercept is administered on multiple days starting on the same day as the administration of the composition. In some embodiments, a first administration of etanercept occurs on the same day as the administration of the composition (Day 1) followed by an administration between about Day 2 to Day 6, optionally where etanercept is not administered on consecutive days. In some embodiments, a first administration of etanercept occurs on the same day as the administration of the composition (Day 1) followed by an administration between about Day 5 to Day 10, optionally where etanercept is not administered on consecutive days. In some embodiments, a first administration of etanercept occurs on the same day as the administration of the composition (Day 1) followed by an administration between about Day 9 to Day 14, optionally where etanercept is not administered on consecutive days. In some embodiments, a first administration of etanercept occurs on the same day as the administration of the composition (Day 1) followed by an administration between about Day 3 to Day 5. In some embodiments, a first administration of etanercept occurs on the same day as the administration of the composition (Day 1) followed by an administration between about Day 7 to Day 9. In some embodiments, a first administration of etanercept occurs on the same day as the administration of the composition (Day 1) followed by an administration between about Day 10 to Day 12. In some embodiments, a first administration of etanercept occurs on the same day as the administration of the composition (Day 1) followed by an administration on Day 4. In some embodiments, a first administration of etanercept occurs on the same day as the administration of the composition (Day 1) followed an administration on Day 8. In some embodiments, a first administration of etanercept occurs on the same day as the administration of the composition (Day 1) followed an administration on Day 11. In some embodiments, etanercept is administered at a dose of about 10 mg to 100 mg, about 15 mg to 95 mg, about 20 mg to 90 mg, about 25 mg to 85 mg, about 30 mg to 80 mg, about 35 mg to 75 mg, about 40 mg to 70 mg, about 45 mg to 65 mg; and/or about 50 mg to 60 mg. In some embodiments, etanercept is administered at a dose of about 50 mg on the same day as the administration of the composition (Day 1). In some embodiments, etanercept is administered at a dose of about 25 mg for any administration occurring on Day 2 or after. In some embodiments, etanercept administration is intravenous (IV) and/or subcutaneous. In some embodiments, the first etanercept administration is a different administration method than subsequent etanercept administrations. In particular examples, the first etanercept administration is IV and the subsequent etanercept administrations are subcutaneous.

### Anti-Thymocyte Globulin Binding Moiety

In some embodiments, the disclosure provides for methods of treating a subject with an anti-thymocyte globulin binding moiety, such as the subject having diabetes who received an organ transplant. In some embodiments, the methods disclosed comprise treating a subject having diabetes, comprising treating the subject with 0.05-3 mg/kg anti-thymocyte globulin binding moiety and subsequently treating the subject with a composition comprising a population of cells, wherein the population comprises a plurality of cells that express NKX6.1 and ISL1.

As used herein, “anti-thymocyte globulin binding moiety” refers to a compound that substantially reduces immune competence in patients with functioning immune systems. In particular embodiments, anti-thymocyte globulin binding moieties reduce the number of circulating T-lymphocytes and their precursors, thymocytes. A non-limiting example of an anti-thymocyte globulin binding moiety is Anti-thymocyte globulin (ATG). Some commercial ATG are THYMOGLOBULIN®, grafalon, and Atgam. THYMOGLOBULIN® and grafalon are rabbit-derived antibodies to human T cells while Atgam is an equine derived antibody to human T cells. THYMOGLOBULIN® is also referred to as “antithymocyte globulin [rabbit].” Additional non-limiting examples of anti-thymocyte globulin binding moieties are antibodies to the a chain of the IL-2 receptor (CD25) on T cells (anti-IL2Rα antibodies), i.e., basiliximab and daclizumab. In some embodiments, the anti-thymocyte globulin binding moiety is an antibody.

A “binding moiety” as used herein, includes antibodies, antibody-like proteins, and binding peptides. In some embodiments, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. The term “antibody” includes polyclonal and monoclonal antibodies, including intact antibodies and functional (antigen-binding) antibody fragments thereof, including fragment antigen binding (Fab) fragments, F(ab′) 2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD. The antibody may target a single target or may target multiple targets. The term “antibody-like proteins” encompasses proteins such as DARPins, affibodies, anticalins, and adnectins.

In some embodiments of the method, the subject receives multiple doses of the anti-thymocyte globulin binding moiety prior to being treated with the composition comprising a population of cells. In some embodiments, the subject is administered a dose of 0.05-3 mg/kg anti-thymocyte globulin binding moiety within 72-24 hours prior to being treated with the composition comprising a population of cells. In some embodiments, the first dose is 0.1-1.25, 0.2-0.8, 0.2-0.7, 0.2-0.6, 0.3-0.6, 0.4-0.6, or 0.45-0.55 mg/kg (e.g., 0.25 or 0.5 mg/kg) anti-thymocyte globulin binding moiety. In some embodiments, the subject is administered a second dose of anti-thymocyte globulin binding moiety at a dose of 0.25-2, 0.25-1.5, 0.25-1.25, 0.5-1.5, 0.5-1.25, 0.75-1.25, 0.9-1.1 (e.g., 0.5 or 1.0 mg/kg). In some embodiments, the subject is administered a third dose of anti-thymocyte globulin binding moiety at a dose of 0.25-3, 0.5-2.5, 0.5-2, 1-2, 1.25-1.75, 0.25-1, or 0.5-0.8 (e.g., 0.5 or 1.0 mg/kg). In some embodiments, the subject is administered a dose of anti-thymocyte globulin binding moiety within 72-60, 72-48, 72-36, 72-24, 60-48, 60-36, 60-24, 48-36, 48-24, or 36-24 hours prior to being treated with the composition comprising a population of cells.

In some embodiments, the anti-thymocyte globulin moiety is administered via infusion. In some embodiments, the infusion is via a high-flow vein. In some embodiments, the infusion lasts for 2-20 hours. In some embodiments, the infusion lasts for 3-18 hours. In some embodiments, the infusion lasts for 4-15 hours. In some embodiments, the infusion lasts for 5-13 hours. In some embodiments, the infusion lasts for 6-12 hours. In some embodiments, the infusion lasts for 7-11 hours. In some embodiments, the infusion lasts for 8-10 hours. In some embodiments, the infusion lasts for 9-11 hours.

In some embodiments, the subject is not administered any anti-thymocyte globulin moiety for a resting period between doses of the anti-thymocyte globulin moiety. In some embodiments, the resting period is between 1-20 hours. In some embodiments, the resting period is between 6-15 hours. In some embodiments, the resting period is between 12-24 hours. In some embodiments, the resting period is between 10-20 hours. In some embodiments, the resting period is between 1-12 hours. In some embodiments, the resting period is between 2-11 hours. In some embodiments, the resting period is between 3-10 hours. In some embodiments, the resting period is between 4-9 hours. In some embodiments, the resting period is between 5-8 hours. In some embodiments, the resting period is between 5-7 hours. In some embodiments, the resting period is between 5-15 hours. In some embodiments, the resting period is between 5-20 hours. In some embodiments, the resting period is between 8-10 hours. In some embodiments, the resting period is between 9-14 hours.

In some embodiments, the population of cells is administered to the subject on Day 1, and wherein the first dose of the anti-thymocyte globulin binding moiety administration protocol begins on Day −5, Day −4, Day −3, Day −2, or Day −1. In some embodiments, the first dose of the anti-thymocyte globulin binding moiety administration protocol begins on Day −2. In some embodiments, no anti-thymocyte globulin binding moiety is infused on Day 1. In some embodiments, the first dose of the anti-thymocyte globulin binding moiety administration protocol is infused over a period of 6-12 hours. In some embodiments, any subsequent dose of the anti-thymocyte globulin binding moiety administration protocol, if any, is infused over a period of 6-12 hours. In some embodiments, there are at least 6 hours between the end of one anti-thymocyte globulin binding moiety infusion and the start of a subsequent anti-thymocyte globulin binding moiety infusion.

In some embodiments, the total dose of anti-thymocyte globulin binding moiety at the end of the anti-thymocyte globulin binding moiety administration protocol is between 1-15, 1-12, 1-10, 1-8, 1-5, 1-3, 3-15, 3-10, 3-8, 3-5, 5-15, 5-10, 5-8, 8-15, or 8-12 mg/kg. In some embodiments, the total dose of anti-thymocyte globulin binding moiety at the end of the anti-thymocyte globulin binding moiety administration protocol is between 1 mg/kg to 10 mg/kg. In some embodiments, the total dose of anti-thymocyte globulin binding moiety at the end of the anti-thymocyte globulin binding moiety administration protocol is between 2 mg/kg to 9 mg/kg. In some embodiments, the total dose of anti-thymocyte globulin binding moiety at the end of the anti-thymocyte globulin binding moiety administration protocol is between 3 mg/kg to 8 mg/kg. In some embodiments, the total dose of anti-thymocyte globulin binding moiety at the end of the anti-thymocyte globulin binding moiety administration protocol is between 4 mg/kg to 7 mg/kg. In some embodiments, the total dose of anti-thymocyte globulin binding moiety at the end of the anti-thymocyte globulin binding moiety administration protocol is between 5 mg/kg to 6 mg/kg. In some embodiments, the total dose of the anti-thymocyte globulin binding moiety administration is 6 mg/kg. In some embodiments, the anti-thymocyte globulin binding moiety is administered at a total dose of less than 6.0 mg/kg (e.g., 1-5.5 mg/kg, 1-4 mg/kg, 2-5.5 mg/kg, 2-4 mg/kg, 1-2 mg/kg, 2.5-3.5 mg/kg, 2.8-3.2 mg/kg, 2.9-3.1 mg/kg or around 3.0 mg/kg). In particular embodiments, the total dose of the anti-thymocyte globulin binding moiety is 3 mg/kg. In some embodiments, the method comprises administering an anti-thymocyte globulin binding moiety in two doses, three doses, four doses, or five doses. In particular embodiments, the anti-thymocyte globulin binding moiety is administered in three doses. In some embodiments, each dose comprises a greater amount of the anti-thymocyte globulin binding moiety than the previous dose. In some embodiments, the anti-thymocyte globulin binding moiety is administered over the course of three doses, wherein the first dose is 0.25-0.75 mg/kg (e.g., 0.5 mg/kg). In some embodiments, the second dose is 0.75-1.25 mg/kg (e.g., 1.0 mg/kg). In some embodiments, the third dose is 1.25-1.75 mg/kg (e.g., 1.5 mg/kg). In some embodiments, the anti-thymocyte globulin binding moiety is administered 5-14 hours, 5-10 hours, 6-10 hours, 5-8 hours, or 5.5-6.5 hours after the last dose of the anti-thymocyte globulin binding moiety.

In some embodiments, the anti-thymocyte globulin binding moiety administration protocol includes dosing the first dose of anti-thymocyte globulin binding moiety administration at a lower dose than any subsequent dose of anti-thymocyte globulin binding moiety. For clarity, “the first dose” does not mean that the subject never received a dose of an anti-thymocyte globulin binding moiety in the past. Rather, “the first dose” means the first dose of an anti-thymocyte globulin binding moiety as part of an immunosuppression regimen in association with treatment of the subject with any of the cells or cell populations described herein. In some embodiments, the first dose of anti-thymocyte globulin binding moiety is 0.1-2, 0.1-1.5, 0.1-1, 0.1-0.7, 0.1-0.5, 0.1-0.3, 0.3-1.5, 0.3-1, 0.3-0.7, 0.4-0.6, 0.6-1.5, or 0.6-1.2 mg/kg. In some embodiments, the first dose of anti-thymocyte globulin binding moiety is between 0.1 to 1.0 mg/kg. In some embodiments, the first dose of anti-thymocyte globulin binding moiety is 0.5 mg/kg. In some embodiments, the first dose is 0.4-0.6 mg/kg. In some embodiments, a second dose of anti-thymocyte globulin binding moiety is higher than the first anti-thymocyte globulin binding moiety dose, but lower than any subsequent dose of anti-thymocyte globulin binding moiety. In some embodiments, the second dose of anti-thymocyte globulin binding moiety is 1.0 mg/kg. In some embodiments, the second dose of anti-thymocyte globulin binding moiety is 0.1-3, 0.1-2, 0.1-1.5, 0.1-1, 0.1-0.5, 0.5-3, 0.5-2.5, 0.5-2, 0.5-1.5, 0.5-1, 0.8-1.2, 1-3, 1-2, or 1-1.5 mg/kg. In some embodiments, the second dose is 0.8-1.2 mg/kg. In some embodiments, the third dose of anti-thymocyte globulin binding moiety is higher than the second dose of anti-thymocyte globulin binding moiety and lower than or the same as any subsequent dose of anti-thymocyte globulin binding moiety. In some embodiments, the third dose of anti-thymocyte globulin binding moiety is 0.1-4, 0.1-4, 0.1-2, 0.1-1.5, 0.1-1, 0.1-0.5, 0.5-4, 0.5-3, 0.5-2, 0.5-1.5, 0.5-1, 1-4, 1-3, 1-2, 1-1.5 or 1.25-1.75 mg/kg. In some embodiments, the third dose is 1.25-1.75 mg/kg. In some embodiments, the third dose of anti-thymocyte globulin binding moiety is 1.5 mg/kg.

In some embodiments, the anti-thymocyte globulin binding moiety administration protocol includes monitoring of total white blood cell (WBC) and platelet counts of the subject following a dose of the anti-thymocyte globulin binding moiety and reducing any subsequent dose of anti-thymocyte globulin binding moiety if the WBC count is between 2,000 and 3,000 cells/mm³. In some embodiments, the anti-thymocyte globulin binding moiety administration protocol includes monitoring of total white blood cell (WBC) and platelet counts of the subject following a dose of the anti-thymocyte globulin binding moiety and reducing any subsequent dose of anti-thymocyte globulin binding moiety if the platelet count is between 50,000 and 75,000 cells/mm³. Reducing any subsequent dose of anti-thymocyte globulin binding moiety includes reducing the dosing by at least 10%, 20%, 30%, 40%, 50%, 60%, 70%, 80%, 90% or 100% of the previous dose. In some embodiments, reducing any subsequent dose of a anti-thymocyte globulin binding moiety includes reducing the dosing by 10-100%, 10-80%, 10-60%, 10-40%, 10-20%, 30-100%, 30-80%, 30-60%, 40-60%, 45-55%, 50-100%, 50-75%, 70-100%, or 85-100% of the previous dose.

In some embodiments, the anti-thymocyte globulin binding moiety administration protocol includes monitoring of total white blood cell (WBC) and platelet counts of the subject following a dose of the anti-thymocyte globulin binding moiety and ceasing further administration of the anti-thymocyte globulin binding moiety if the WBC count falls below 2,000 cells/mm³. In some embodiments, the anti-thymocyte globulin binding moiety administration protocol includes monitoring of total white blood cell (WBC) and platelet counts of the subject following a dose of the anti-thymocyte globulin binding moiety and stopping the anti-thymocyte globulin binding moiety administration if the platelet count falls below 50,000 cells/mm³.

In some embodiments, if the subject develops an allergic reaction to the anti-thymocyte globulin binding moiety (e.g., anaphylaxis), the anti-thymocyte globulin binding moiety infusion is terminated. In some embodiments, the allergic reaction is anaphylaxis. In some embodiments, the subject is treated for the anaphylaxis by administering epinephrine intramuscularly and/or other resuscitative measures including oxygen, IV fluids, antihistamines, corticosteroids, pressor amines, and airway management.

In some embodiments, if any of the subjects disclosed herein (e.g., a subject having Type 1 Diabetes and having previously had an organ transplant such as a kidney transplant) was previously administered an anti-thymocyte globulin binding moiety (e.g., in connection with a prior organ (e.g., kidney) transplant) within 1 year, within 2 years, within 3 years, within 4 years, within 5 years, within 6 years, within 8 years, or within 10 years prior to administration of any of the cell compositions disclosed herein (e.g., any of the SC-islet compositions disclosed herein), then the subject is not administered an anti-thymocyte globulin binding moiety in connection with administering any of the cell compositions disclosed herein (e.g., any of the SC-islet compositions disclosed herein). In some embodiments, if any of the subjects disclosed herein (e.g., a subject having Type 1 Diabetes and having previously had an organ transplant such as a kidney transplant) was previously administered an anti-thymocyte globulin binding moiety at any point in the subject's lifetime (e.g., in connection with a prior organ (e.g., kidney) transplant), then the subject is not administered an anti-thymocyte globulin binding moiety in connection with administering any of the cell compositions disclosed herein (e.g., any of the SC-islet compositions disclosed herein). In some embodiments, if the subject was previously administered an anti-thymocyte globulin binding moiety at any point in the subject's lifetime, then the subject is administered one or more immunosuppressive agents that are not an anti-thymocyte globulin binding moiety at or around the same time that the cell composition is administered to the subject. In some embodiments, if the subject is not administered an anti-thymocyte globulin binding moiety in connection with the administration of the cell composition, then the subject is administered an alternative immunosuppressive agent (e.g., any of the other immunosuppressive agents disclosed herein, such as basiliximab or alemtuzumab), In some embodiments, the one or more immunosuppressive agents is an antibody or antibody fragment (e.g., an scFv, sdAb, sdFv, or nanobody). In some embodiments, the antibody is a human or humanized antibody or antibody fragment. In some embodiments, the antibody or antibody fragment is not non-human or non-humanized. In some embodiments, the one or more immunosuppressive agents is an anti-CD25 antibody or antibody fragment. In some embodiments, the one or more immunosuppressive agents is basiliximab. In some embodiments, the one or more immunosuppressive agents is an anti-CD52 antibody or antibody fragment. In some embodiments, the one or more immunosuppressive agents is alemtuzumab. In some embodiments, the basiliximab is administered at a dose of 10-30 mg, 15-25 mg, 18-22 mg, or 19-21 mg. In some embodiments, the one or more immunosuppressive agents is administered on the same day as the composition. In some embodiments, the method further comprises the step of administering the one or more immunosuppressive agents to the subject 2-10 days, 2-8 days, 2-7 days, 2-5 days, 3-8 days, 3-6 days, 4-6 days, 4 days, or 5 days after the composition comprising a population of stem cell-derived islet cells. In some embodiments, the subject administered the cell composition and the one or more immunosuppressive agents was not previously administered a composition comprising a population of stem cell-derived islet cells.

### Premedications

Some immunosuppression-induction agents, such as anti-thymocyte globulin binding moiety, are known to cause cytokine release-related fever and chills in some subjects. In some embodiments, premedication is administered in order to minimize the cytokine release reaction incidence and/or intensity. The premedication can be administered to the subject having diabetes who received an organ transplant. As used herein, “premedications” are any medications administered in order to minimize the cytokine release reaction incidence and/or intensity. In some embodiments, the premedication is a pain reducer (e.g., acetaminophen or an NSAID). In some embodiments, the premedication is an antihistamine (e.g., azelastine, brompheniramine, cetirizine, chlorpheniramine, desloratadine, or diphenhydramine). In some embodiments, the premedication is a corticosteroid (e.g., cortisone, hydrocortisone, prednisone prednisolone; methylprednisolone; dexamethasone or betamethasone). In some embodiments, the premedication is a blood thinner or claudication therapy (e.g., pentoxifylline or cilostazol). Examples of premedications are acetaminophen, antihistamine, corticosteroids, and/or pentoxifylline. Premedications can be administered in any combination of medication. Premedications can be administered in any combination of timing, i.e. before, concurrently, and/or after anti-thymocyte globulin binding moiety treatment administration. In some embodiments, the premedications are administered before the anti-thymocyte globulin binding moiety treatment administration. In some embodiments, the premedications are administered during anti-thymocyte globulin binding moiety treatment administration. In some embodiments, the premedications are administered after anti-thymocyte globulin binding moiety treatment administration. In some embodiments, the premedications are administered before and during anti-thymocyte globulin binding moiety treatment administration. In some embodiments, the premedications are administered before and after anti-thymocyte globulin binding moiety treatment administration. In some embodiments, the premedications are administered during and after anti-thymocyte globulin binding moiety treatment administration. In some embodiments, the premedications are administered before, during, and after anti-thymocyte globulin binding moiety treatment administration. If more than one premedication is being administered, the premedications do not have to be administered at the same time. In some embodiments, the more than one premedications are administered together. In other embodiments, the more than one premedications are administered contemporaneously. In some embodiments, the one or more premedications is administered 0.25-6 hours, 0.25-4 hours, 0.25-3 hours, 0.25-2 hours, 0.25-1 hours, 0.25-0.75 hours, 0.5-6 hours, 0.5-4 hours, 0.5-3 hours, 0.5-2 hours, 0.5-1 hours, 1-6 hours, 1-4 hours, 1-3 hours, 1-2 hours, 2-6 hours, or 2-4 hours prior to administration of a treatment (e.g., the first dose) with an anti-thymocyte globulin binding moiety. In some embodiments, the one or more premedications are administered 0.25-1.5 hours prior to administration of a treatment (e.g., the first dose) with an anti-thymocyte globulin binding moiety. In some embodiments, the one or more premedications are administered during treatment (e.g., during infusion) of the anti-thymocyte globulin binding moiety. In some embodiments, the one or more premedications are administered after 0.25-0.75%, 0.3-0.6%, 0.4-0.6%, 0.45-0.55%, or 0.4-0.7% of an anti-thymocyte globulin binding moiety dose has been administered (e.g., during infusion).

In some embodiments, the premedications dosing comprise a pharmaceutically acceptable amount of any one of or combination of acetaminophen, antihistamine, corticosteroids, and/or pentoxifylline.

In some embodiments, acetaminophen is administered as a premedication. In some embodiments, the acetaminophen is administered at a dose ranging from 100 mg to 1200 mg. In some embodiments, the acetaminophen is administered at a dose ranging from 200 mg to 1100 mg. In some embodiments, the acetaminophen is administered at a dose ranging from 300 mg to 1000 mg. In some embodiments, the acetaminophen is administered at a dose ranging from 400 mg to 900 mg. In some embodiments, the acetaminophen is administered at a dose ranging from 500 mg to 800 mg. In some embodiments, the acetaminophen is administered at a dose ranging from 600 mg to 700 mg. In some embodiments, the acetaminophen is administered at around 650 mg.

In some embodiments, the acetaminophen is administered by mouth (PO). In some embodiments, the acetaminophen is administered by rectum (PR). In some embodiments, the acetaminophen is administered at least 2 hours before the anti-thymocyte globulin binding moiety treatment administration. In some embodiments, the acetaminophen is administered at least 1 hour before the anti-thymocyte globulin binding moiety treatment administration. In some embodiments, the acetaminophen is administered at least 30 minutes before the anti-thymocyte globulin binding moiety treatment administration. In some embodiments, the acetaminophen is administered during the anti-thymocyte globulin binding moiety treatment administration. In some embodiments, the acetaminophen is administered after at least 10% of an anti-thymocyte globulin binding moiety treatment dose has been administered. In some embodiments, the acetaminophen is administered after at least 20% of an anti-thymocyte globulin binding moiety treatment dose has been administered. In some embodiments, the acetaminophen is administered after at least 30% of an anti-thymocyte globulin binding moiety treatment dose has been administered. In some embodiments, the acetaminophen is administered after at least 40% of an anti-thymocyte globulin binding moiety treatment dose has been administered. In some embodiments, the acetaminophen is administered after at least 50% of an anti-thymocyte globulin binding moiety treatment dose has been administered. In some embodiments, the acetaminophen is administered after at least 60% of an anti-thymocyte globulin binding moiety treatment dose has been administered. In some embodiments, the acetaminophen is administered after at least 70% of an anti-thymocyte globulin binding moiety treatment dose has been administered. In some embodiments, the acetaminophen is administered after at least 80% of an anti-thymocyte globulin binding moiety treatment dose has been administered. In some embodiments, the acetaminophen is administered after at least 90% of an anti-thymocyte globulin binding moiety treatment dose has been administered. In some embodiments, the acetaminophen is administered before and during the anti-thymocyte globulin binding moiety treatment administration. In some embodiments, a first dose of acetaminophen is administered at least 30 minutes before the start of the anti-thymocyte globulin binding moiety treatment and a second dose of acetaminophen is administered after at least 25% of the anti-thymocyte globulin binding moiety treatment has been administered. In some embodiments, a first dose of acetaminophen is administered at least 30 minutes before the start of the anti-thymocyte globulin binding moiety treatment and a second dose of acetaminophen is administered after at least 40% of the anti-thymocyte globulin binding moiety treatment has been administered.

In some embodiments, the premedications include diphenhydramine. In some embodiments, the diphenhydramine is administered at a dose ranging from 10 mg to 100 mg. In some embodiments, the diphenhydramine is administered at a dose ranging from 20 mg to 90 mg. In some embodiments, the diphenhydramine is administered at a dose ranging from 30 mg to 80 mg. In some embodiments, the diphenhydramine is administered at a dose ranging from 40 mg to 70 mg. In some embodiments, the diphenhydramine is administered at a dose ranging from 50 mg to 60 mg. In some embodiments, the diphenhydramine is administered at around 50 mg.

In some embodiments, the diphenhydramine is administered by mouth (PO). In some embodiments, the diphenhydramine is administered at least 2 hours before the anti-thymocyte globulin binding moiety treatment administration. In some embodiments, the diphenhydramine is administered at least 1 hour before the anti-thymocyte globulin binding moiety treatment administration. In some embodiments, the diphenhydramine is administered at least 30 minutes before the anti-thymocyte globulin binding moiety treatment administration. In some embodiments, the diphenhydramine is administered during the anti-thymocyte globulin binding moiety treatment administration. In some embodiments, the diphenhydramine is administered after at least 10% of an anti-thymocyte globulin binding moiety treatment dose has been administered. In some embodiments, the diphenhydramine is administered after at least 20% of an anti-thymocyte globulin binding moiety treatment dose has been administered. In some embodiments, the diphenhydramine is administered after at least 30% of an anti-thymocyte globulin binding moiety treatment dose has been administered. In some embodiments, the diphenhydramine is administered after at least 40% of an anti-thymocyte globulin binding moiety treatment dose has been administered. In some embodiments, the diphenhydramine is administered after at least 50% of an anti-thymocyte globulin binding moiety treatment dose has been administered. In some embodiments, the diphenhydramine is administered after at least 60% of an anti-thymocyte globulin binding moiety treatment dose has been administered. In some embodiments, the diphenhydramine is administered after at least 70% of an anti-thymocyte globulin binding moiety treatment dose has been administered. In some embodiments, the diphenhydramine is administered after at least 80% of an anti-thymocyte globulin binding moiety treatment dose has been administered. In some embodiments, the diphenhydramine is administered after at least 90% of an anti-thymocyte globulin binding moiety treatment dose has been administered. In some embodiments, the diphenhydramine is administered before and during the anti-thymocyte globulin binding moiety treatment administration. In some embodiments, a first dose of diphenhydramine is administered at least 30 minutes before the start of the anti-thymocyte globulin binding moiety treatment and a second dose of diphenhydramine is administered after at least 25% of the anti-thymocyte globulin binding moiety treatment has been administered. In some embodiments, a first dose of diphenhydramine is administered at least 30 minutes before the start of the anti-thymocyte globulin binding moiety treatment and a second dose of diphenhydramine is administered after at least 40% of the anti-thymocyte globulin binding moiety treatment has been administered.

In some embodiments, the premedications include methylprednisolone. In some embodiments, the methylprednisolone is administered at a dose ranging from 0.1 mg/kg to 5.0 mg/kg. In some embodiments, the methylprednisolone is administered at a dose ranging from 0.2 mg/kg to 4.5 mg/kg. In some embodiments, the methylprednisolone is administered at a dose ranging from 0.3 mg/kg to 4.0 mg/kg. In some embodiments, the methylprednisolone is administered at a dose ranging from 0.4 mg/kg to 3.5 mg/kg. In some embodiments, the methylprednisolone is administered at a dose ranging from 0.5 mg/kg to 3.0 mg/kg. In some embodiments, the methylprednisolone is administered at a dose ranging from 0.6 mg/kg to 2.5 mg/kg. In some embodiments, the methylprednisolone is administered at a dose ranging from 0.7 mg/kg to 2.0 mg/kg. In some embodiments, the methylprednisolone is administered at a dose ranging from 0.8 mg/kg to 1.5 mg/kg. In some embodiments, the methylprednisolone is administered at a dose ranging from 0.9 mg/kg to 1.25 mg/kg. In some embodiments, the methylprednisolone is administered at a dose of around 1.0 mg/kg.

In some embodiments, the methylprednisolone is administered intravenously (IV). In some embodiments, the methylprednisolone is administered at least 2 hours before the anti-thymocyte globulin binding moiety treatment administration. In some embodiments, the methylprednisolone is administered at least 1 hour before the anti-thymocyte globulin binding moiety treatment administration. In some embodiments, the methylprednisolone is administered at least 30 minutes before the anti-thymocyte globulin binding moiety treatment administration. In some embodiments, the methylprednisolone is administered as needed during the anti-thymocyte globulin binding moiety treatment administration.

In some embodiments, the premedications include pentoxifylline. In some embodiments, the pentoxifylline is administered at a dose ranging from 50 mg to 1000 mg. In some embodiments, the pentoxifylline is administered at a dose ranging from 75 mg to 900 mg. In some embodiments, the pentoxifylline is administered at a dose ranging from 100 mg to 800 mg. In some embodiments, the pentoxifylline is administered at a dose ranging from 200 mg to 700 mg. In some embodiments, the pentoxifylline is administered at a dose ranging from 300 mg to 600 mg. In some embodiments, the pentoxifylline is administered at a dose ranging from 400 mg to 500 mg. In some embodiments, the pentoxifylline is administered at around 400 mg.

In some embodiments, the pentoxifylline is administered by mouth (PO). In some embodiments, the pentoxifylline is administered once a day (QD). In some embodiments, the pentoxifylline is administered twice a day (BID). In some embodiments, the pentoxifylline is administered three times a day (TID). In some embodiments, the pentoxifylline is administered starting on Day −4 continuing through Day 11. In some embodiments, the pentoxifylline is administered starting on Day −3 continuing through Day 10. In some embodiments, the pentoxifylline is administered starting on Day −2 continuing through Day 9. In some embodiments, the pentoxifylline is administered on Day −2 at least 1 hour before the anti-thymocyte globulin binding moiety treatment administration and pentoxifylline administration continues through Day 8.

### Immune Response Modulators

In some cases, the methods of treatment provided herein can comprise administering one or more immune response modulators for modulating or reducing transplant rejection response or other immune response against the administered cells to a subject, such as the subject having diabetes who received an organ transplant. In some embodiments, the immune response modulator is not or does not comprise a steroid. In some cases, the immune response modulator comprises a steroid such as corticosteroid. Examples of immune response modulators that can be used in the methods can include purine synthesis inhibitors (e.g., azathioprine and mycophenolic acid), pyrimidine synthesis inhibitors (e.g., leflunomide and teriflunomide), antifolate (e.g., methotrexate), tacrolimus, ciclosporin, pimecrolimus, abetimus, gusperimus, lenalidomide, pomalidomide, thalidomide, PDE4 inhibitor, apremilast, anakinra, sirolimus, everolimus, ridaforolimus, temsirolimus, umirolimus, zotarolimus, anti-thymocyte globulin antibodies, anti-lymphocyte globulin antibodies, CTLA-4 and fragments thereof and/or fusion proteins thereof (e.g., abatacept and belatacept), TNF inhibitor (e.g., etanercept and pegsunercept, aflibercept, alefacept, rilonacept), antibodies against complement component 5 (e.g., eculizumab), anti-TNF antibodies (e.g., adalimumab, afelimomab, certolizumab pegol, golimumab, infliximab, and nerelimomab), antibodies against Interleukin 5 (e.g., mepolizumab), anti-Ig E antibodies (e.g., omalizumab), anti-Interferon antibodies (e.g., faralimomab), anti-IL-6 antibodies (e.g., elsilimomab), antibodies against IL-12 and IL-23 (e.g., lebrikizumab and ustekinumab), anti-IL-17A antibodies (e.g., secukinumab), anti-CD3 antibodies (e.g., muromonab-CD3, otelixizumab, teplizumab, and visilizumab), anti-CD4 antibodies (e.g., clenoliximab, keliximab, and zanolimumab), anti-CD11a antibodies (e.g., efalizumab), anti-CD18 antibodies (e.g., erlizumab), anti-CD20 antibodies (e.g., obinutuzumab, rituximab, ocrelizumab and pascolizumab), anti-CD23 antibodies (e.g., gomiliximab and lumiliximab), anti-CD40 antibodies (e.g., teneliximab and toralizumab), antibodies against CD62L/L-selectin (e.g., aselizumab), anti-CD80 antibodies (e.g., galiximab), anti-CD147/Basigin antibodies (e.g., gavilimomab), anti-CD154 antibodies (e.g., ruplizumab), anti-BLyS antibodies (e.g., belimumab and blisibimod), anti-CTLA-4 antibodies (e.g., ipilimumab and tremelimumab), anti-CAT antibodies (e.g., bertilimumab, lerdelimumab, and metelimumab), anti-Integrin antibodies (e.g., natalizumab), antibodies against Interleukin-6 receptor (e.g., tocilizumab), anti-LFA-1 antibodies (e.g., odulimomab), antibodies against IL-2 receptor/CD25 (e.g., basiliximab, daclizumab, and inolimomab), antibodies against T-lymphocyte (Zolimomab aritox) (e.g., atorolimumab, cedelizumab, fontolizumab, maslimomab, morolimumab, pexelizumab, reslizumab, rovelizumab, siplizumab, talizumab, telimomab aritox, vapaliximab, and vepalimomab), antibodies against CD52 (e.g., alemtuzumab), blockers of inosine monophosphate dehydrogenase (IMPDH) (e.g., mycophenolate mofetil), inhibitors of cell emigration (e.g., FTY720), or any combination thereof. In some embodiments, the immune response modulator comprises tacrolimus, cyclosporine, mycophenolate mofetil, azathioprine, everolimus, sirolimus, abatacept, belatacept, antithymocyte globulin, alemtuzumab, rituximab, basiliximab, daclizumab, muromonab-CD3, efalizumab, and/or FTY720.

In some embodiments, the immune response modulator is administered concurrently with the composition comprising the population of cells. In some embodiments, the immune response modulator is administered prior to or subsequent to the administration of the composition comprising the population of cells. In some embodiments, the immune response modulator is not a steroid. In some embodiments, the immune response modulator comprises azathioprine, mycophenolic acid, leflunomide, teriflunomide, methotrexate, tacrolimus, ciclosporin, pimecrolimus, abetimus, gusperimus, lenalidomide, pomalidomide, thalidomide, PDE4 inhibitor, apremilast, anakinra, sirolimus, everolimus, ridaforolimus, temsirolimus, umirolimus, zotarolimus, anti-thymocyte globulin antibodies, anti-lymphocyte globulin antibodies, CTLA-4, abatacept, belatacept, etanercept, pegsunercept, aflibercept, alefacept, rilonacept, eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, atorolimumab, cedelizumab, fontolizumab, maslimomab, morolimumab, pexelizumab, reslizumab, rovelizumab, siplizumab, talizumab, telimomab aritox, vapaliximab, vepalimomab, alemtuzumab, mycophenolate mofetil, FTY720, or any combination thereof. In some embodiments, the immune response modulator comprises tacrolimus, cyclosporine, mycophenolate mofetil, azathioprine, everolimus, sirolimus, abatacept, belatacept, antithymocyte globulin, alemtuzumab, rituximab, basiliximab, daclizumab, muromonab-CD3, efalizumab, FTY720, or any combination thereof. In some embodiments, the immune response modulator comprises tacrolimus, cyclosporine, mycophenolate mofetil, azathioprine, everolimus, sirolimus, abatacept, belatacept, antithymocyte globulin, alemtuzumab, rituximab, basiliximab, daclizumab, muromonab-CD3, efalizumab, FTY720, or any combination thereof.

### Blood Thinners

In some embodiments, the disclosure provides for a method of treating a subject with any of the cell compositions disclosed herein and a blood thinner, such as the subject having diabetes who received an organ transplant. In some embodiments, the blood thinner is any one or more of pentoxifylline, aspirin, ticagrelor, warfarin, prasugrel, apixaban, clopidogrel, dabigatran, edoxaban, rivaroxaban, heparin, or a low molecular weight heparin (e.g., enoxaparin or dalteparin). In some embodiments, the blood thinner is aspirin. In some embodiments, the blood thinner is enoxaparin. In some embodiments, the blood thinner is heparin. In some embodiments, the subject is administered a blood thinner contemporaneously with the administration of the cell composition. In some embodiments, the subject is administered heparin when being administered the cell composition. In some embodiments, the heparin is administered at a dose of 1-200 U/kg, 1-150 U/kg, 1-100 U/kg, 1-75 U/kg, 1-50 U/kg, 1-10 U/kg, 1-5 U/kg, 30-300 U/kg, 30-150 U/kg, 30-100 U/kg, 30-75 U/kg, 30-50 U/kg, 60-200 U/kg, 60-150 U/kg, 60-100 U/kg or 60-80 U/kg. In some embodiments, the cell composition is administered with the heparin at a dose of 30-150 U/kg, 30-100 U/kg or 30-75 U/kg. In some embodiments, within the 1-10, 1-8, 1-6, 1-5, 1-4, 1-3, or 3-5 hours after administration of the cell composition, the subject is administered heparin at a dose of 1-25, 1-10, or 1-5 U/kg. In some embodiments, the subject is administered heparin for the first 12-72, 12-60, 12-48, 12-36, 12-24, 24-72, 24-60, 24-48, 24-36, 36-72, 36-60, 36-48, or 45-53 hours (e.g., around days) after being administered the cell composition. In some embodiments, the subject is not administered heparin past the first 12-72, 12-60, 12-48, 12-36, 12-24, 24-72, 24-60, 24-48, 24-36, 36-72, 36-60, 36-48, or 45-53 hours (e.g., around two days) after being administered the cell composition. In some embodiments, the subject is administered heparin intravenously or subcutaneously. In some embodiments, the subject is administered enoxaparin at a dose of 1-100, 1-75, 1-50, 1-25, 20-100, 20-75, 20-50, 20-40, 50-100, or 50-75 mg of enoxaparin. In some embodiments, the subject is administered enoxaparin intravenously or subcutaneously. In some embodiments, the subject is administered enoxaparin after the subject previously received heparin. In some embodiments, the subject is administered enoxaparin starting 12-72, 12-60, 12-48, 12-36, 12-24, 24-72, 24-60, 24-48, 24-36, 36-72, 36-60, 36-48, or 45-53 hours (e.g., around two days) after the cell composition was administered to the subject. In some embodiments, the subject is administered enoxaparin for 1-20, 1-15, 1-10, 1-8, 1-6, 1-5, 1-3, 5-20, 5-15, 5-10, 5-8 days (e.g., around 7 days). In some embodiments, the subject is administered aspirin at a dose of 1-400, 1-300, 1-200, 1-100, 1-50, 50-400, 50-300, 50-200, or 50-100 mg. In some embodiments, the subject is administered aspirin orally. In some embodiments, the subject is administered aspirin four times a day, three times a day, twice a day, daily, every other day, or as needed.

### Tumor Necrosis Factor-Alpha (TNFα) Inhibitor

In some embodiments, the disclosure provides for a method of treating a subject, such as the subject having diabetes who received an organ transplant, with a TNFα inhibitor and with a composition comprising a population of cells, wherein the population comprises a plurality of cells that express NKX6.1 and ISL1. In some embodiments, the TNFα inhibitor is any of etanercept and pegsunercept, aflibercept, alefacept, rilonacept, adalimumab, afelimomab, certolizumab pegol, golimumab, infliximab, or nerelimomab. In particular embodiments, the TNFα inhibitor is etanercept. Also disclosed herein, in a certain aspect, is a method of treating a subject having diabetes, comprising treating the subject with: a) 10-100 mg of a Tumor Necrosis Factor-alpha (TNFα) inhibitor and b) a composition comprising a population of cells, wherein the population comprises a plurality of cells that express NKX6.1 and ISL1.

In some aspects, the disclosure is directed to a method of treating a subject having diabetes, comprising: a) treating the subject with a composition comprising a population of cells, wherein the population comprises a plurality of cells that express NKX6.1 and ISL1, and b) treating the subject with multiple doses of a Tumor Necrosis Factor-alpha (TNFα) inhibitor. In some embodiments, the subject is treated with 2, 3, 4, 5, 6, or 7 doses of the TNFα inhibitor in the month following the administration of the composition comprising the population of cells. In some embodiments, the subject is treated with the TNFα inhibitor about 3 days, about 4 days, about 5 days, about 6 days, about 7 days, about 8 days, about 9 days, about 10 days, about 11 days, about 12 days, about 13 days, about 14 days, and/or about 15 days following the administration of the composition comprising the population of cells. In some embodiments, the subject is treated with 5-500 mg TNFα inhibitor for each dose of the TNFα inhibitor. In some embodiments, the subject is treated with 5-100 mg TNFα inhibitor for each dose of the TNFα inhibitor. In some embodiments, the subject is treated with 5-75 mg TNFα inhibitor for each dose of the TNFα inhibitor. In some embodiments, the subject is treated with 5-50 mg TNFα inhibitor for each dose of the TNFα inhibitor. In some embodiments, the subject is treated with 10-100 mg TNFα inhibitor for each dose of the TNFα inhibitor. In some embodiments, the subject is treated with 10-75 mg TNFα inhibitor for each dose of the TNFα inhibitor. In some embodiments, the subject is treated with 10-50 mg TNFα inhibitor for each dose of the TNFα inhibitor. In some embodiments, the subject is treated with 30-100 mg TNFα inhibitor for each dose of the TNFα inhibitor. In some embodiments, the subject is treated with 30-75 mg TNFα inhibitor for each dose of the TNFα inhibitor. In some embodiments, the TNFα inhibitor is administered subcutaneously and/or intravenously. In some embodiments, the TNFα inhibitor is selected from the group consisting of etanercept, adalimumab, infliximab, certolizumab pegol, and golimumab. In some embodiments, the subject is administered etanercept. In some embodiments, etanercept is administered on the same day as the administration of the composition comprising the population of cells. In some embodiments, etanercept is administered about one hour before the administration of the composition comprising the population of cells. In some embodiments, etanercept is administered on the same day as the administration of the composition comprising the population of cells (Day 1) followed by further administration on Day 2, Day 3, Day 4, Day 5, Day 6, Day 7, Day 8, Day 9, Day 10, Day 11, Day 12, Day 13, Day 14, and/or Day 15. In some embodiments, etanercept is administered on multiple days starting on the same day as the administration of the composition comprising the population of cells. In some embodiments, a first administration of etanercept occurs on the same day as the administration of the composition comprising the population of cells (Day 1) followed by a second administration of etanercept between Day 2 to Day 6; a third administration of etanercept between Day 5 to Day 10; and/or a fourth administration of etanercept between Day 9 to Day 14. In some embodiments, etanercept is not administered on consecutive days. In some embodiments, a first administration of etanercept occurs on the same day as the administration of the composition comprising the population of cells (Day 1) followed by a second administration between Day 3 to Day 5; a third administration between Day 7 to Day 9; and/or a fourth administration between Day 10 to Day 12. In some embodiments, a first administration of etanercept occurs on the same day as the administration of the composition comprising the population of cells (Day 1) followed by a second administration on Day 4; a third administration on Day 8; and/or a fourth administration on Day 11. In some embodiments, etanercept is administered at a dose ranging from 10 mg to 100 mg. In some embodiments, etanercept is administered at a dose ranging from 15 mg to 95 mg. In some embodiments, etanercept is administered at a dose ranging from 20 mg to 90 mg. In some embodiments, etanercept is administered at a dose ranging from 25 mg to 85 mg. In some embodiments, etanercept is administered at a dose ranging from 30 mg to 80 mg. In some embodiments, etanercept is administered at a dose ranging from 35 mg to 75 mg. In some embodiments, etanercept is administered at a dose ranging from 40 mg to 70 mg. In some embodiments, etanercept is administered at a dose ranging from 45 mg to 65 mg. In some embodiments, etanercept is administered at a dose ranging from 50 mg to 60 mg. In some embodiments, etanercept is administered at a dose of 50 mg on the same day as the administration of the composition comprising the population of cells (Day 1). In some embodiments, etanercept is administered at a dose of 25 mg for any administration occurring on Day 2 or after. In some embodiments, the etanercept administration is intravenous (IV) and/or subcutaneous. In some embodiments, the first etanercept administration is a different administration method than subsequent etanercept administrations. In some embodiments, the first etanercept administration is IV and the subsequent etanercept administrations are subcutaneous.

In some embodiments, the disclosure provides for a method of treating a subject having diabetes by administrating a composition comprising a population of cells, wherein prior to the administration of the composition comprising the population of cells, the subject received insulin at a level of 1-50, 1-40, 1-35, 1-30, 1-25, 1-20, 7-50, 7-40, 7-35, 7-30, 7-25, 7-20, 15-50, 15-40, 15-35, 15-30, 15-25, 15-20, 20-50, 20-40, 20-35, 20-30, 20-25, 25-35 or 25-30 U/day. In some embodiments, prior to the administration of the composition comprising the population of cells, the subject received insulin at a level of 20-35 U/day. Disclosed herein, in a certain aspect, is a method of treating a subject having diabetes through administration of a composition comprising a population of cells, wherein prior to the administration of the composition comprising the population of cells, the subject received infusion of insulin at a level of at least about 20 U/day. In some embodiments, the subject received infusion of insulin at a level of at least about 22 U/day prior to the administration of the composition comprising the population of cells. In some embodiments, the subject received infusion of insulin at a level of at least about 24 U/day prior to the administration of the composition comprising the population of cells. In some embodiments, the subject received infusion of insulin at a level of at least about 26 U/day prior to the administration of the composition comprising the population of cells. In some embodiments, the subject received infusion of insulin at a level of at least about 28 U/day prior to the administration of the composition comprising the population of cells. In some embodiments, the subject received infusion of insulin at a level of at least about 30 U/day prior to the administration of the composition comprising the population of cells. In some embodiments, the subject received infusion of insulin at a level of at least about 32 U/day prior to the administration of the composition comprising the population of cells. In some embodiments, the subject received infusion of insulin at a level of at least about or 34 U/day.

Disclosed herein, in a certain aspect, is a method of treating a subject having diabetes, comprising treating the subject with: a) sirolimus (e.g., 0.01-5000 mg/kg of sirolimus) and b) a composition comprising a population of cells, wherein the population comprises a plurality of cells that express NKX6.1 and ISL1. In some embodiments, the subject is treated with 0.01-1 mg/kg sirolimus. In some embodiments, the subject is treated with 0.02-0.3 mg/kg sirolimus. In some embodiments, the subject is treated with 0.02-0.15 mg/kg sirolimus. In some embodiments, the subject is treated with 0.02-0.12 mg/kg sirolimus. In some embodiments, the subject is treated with 0.02-0.08 mg/kg sirolimus. In some embodiments, the subject is treated with 0.03-0.07 mg/kg sirolimus. In some embodiments, the subject is treated with 0.04-0.15 mg/kg sirolimus. In some embodiments, the subject is treated with or 0.05-2 mg/kg sirolimus. In some embodiments, the sirolimus is administered daily. In some embodiments, the sirolimus is administered orally.

Disclosed herein, in certain aspect, is a method of treating a subject having diabetes, comprising treating the subject with: a) tacrolimus (e.g., 0.001-0.05 mg/kg of tacrolimus) and b) a composition comprising a population of cells, wherein the population comprises a plurality of cells that express NKX6.1 and ISL1. In some embodiments, the subject is treated with 0.001-0.05 mg/kg tacrolimus. In some embodiments, the subject is treated with 0.003-0.03 mg/kg tacrolimus. In some embodiments, the subject is treated with 0.004-0.02 mg/kg tacrolimus. In some embodiments, the subject is treated with 0.006-0.02 mg/kg tacrolimus. In some embodiments, the subject is treated with 0.008-0.02 mg/kg tacrolimus. In some embodiments, the subject is treated with 0.01-0.02 mg/kg tacrolimus. In some embodiments, the subject is treated with 0.009-0.03 mg/kg tacrolimus. In some embodiments, the subject is treated with 0.009-0.02 mg/kg tacrolimus. In some embodiments, the tacrolimus is administered daily. In some embodiments, the tacrolimus is administered orally.

In some embodiments, the disclosure is directed to a method of treating a subject having diabetes, comprising treating the subject with 0.05-3 mg/kg anti-thymocyte globulin binding moiety (e.g., THYMOGLOBULIN®) as described herein, and subsequently treating the subject with a composition comprising a population of cells, wherein the population comprises a plurality of cells that express NKX6.1 and ISL1, and wherein the subject is also administered Tacrolimus and/or Sirolimus. In some embodiments, the administration of Sirolimus is started before the day of the administration of the composition comprising the population of cells (Day 1). In some embodiments, the administration of Sirolimus is started between Day −5 and Day −1. In some embodiments, wherein the administration of Sirolimus starts on Day −2. In some embodiments, wherein the first dose of Sirolimus is 0.05 to 0.2 mg/kg and subsequent doses are 0.05 to 0.1 mg/kg once each morning (qam). In some embodiments, the sirolimus dose is adjusted to maintain a whole blood 24-hour trough target of 10 to 15 ng/mL for the first 3 months after administration of the composition. In some embodiments, the sirolimus dose is adjusted to maintain a whole blood 24-hour trough target of 8 to 12 ng/ml after the first 3 months after administration of the composition. In some embodiments, the administration of tacrolimus is started after the day of the administration of the composition comprising the population of cells (Day 1). In some embodiments, the administration of tacrolimus is started between Day 2 and Day 5 of the administration of the composition. In some embodiments, the administration of tacrolimus is started on Day 2 of the administration of the first pharmaceutical composition. In some embodiments, the tacrolimus is started at a dose of 0.015 mg/kg given even 12 hours. In some embodiments, the tacrolimus dose is adjusted to maintain a whole blood 12-hour trough of 3 to 6 ng/mL. In some embodiments, if the subject develops intolerable or clinically undesirable side-effects related to sirolimus or tacrolimus treatment, the sirolimus or tacrolimus is replaced with an alternative immunosuppressive agent (e.g., mycophenolate mofetil or mycophenolate sodium). In some embodiments, mycophenolate mofetil is dosed between 500 to 1000 mg twice a day (BID) and mycophenolate sodium is dosed between 360 to 720 mg BID.

In some embodiments of any of the methods of treatment disclosed herein, the dose of tacrolimus administered to the subject is adjusted depending on whether or not sirolimus is also being administered to the subject at the same time. In some embodiments, if the subject is also being administered sirolimus, then the tacrolimus dose is adjusted to maintain a whole blood 12-hour trough of 1-12 ng/ml, 1-10 ng/mL, 1-7 ng/mL, 2-8 ng/ml, 2-7 ng/ml, or 3-6 ng/mL. In some embodiments, if the subject is also being administered sirolimus, then the tacrolimus dose is adjusted to maintain a whole blood 12-hour trough of 3-6 ng/mL. In some embodiments, if the subject is not also being administered sirolimus, the tacrolimus dose is adjusted to maintain a whole blood 12-hour trough of 6-12 ng/mL, 7-11 ng/mL, or 8-10 ng/mL for the first 1-6 months, 1-4 months, 1-3 months, 2-4 months or 2.5-3.5 months. In some embodiments, if the subject is not also being administered sirolimus, the tacrolimus dose is adjusted to maintain a whole blood 12-hour trough of 8-10 ng/ml for the first 2.5-3.5 months. In some embodiments, 1-10 months 1-8 months, 1-6 months, 3-10 months, 3-8 months, 3-6 months, or 5.5-6.5 months after the subject was administered the composition comprising a population of stem cell-derived islet cells, and if the subject is not also being administered sirolimus, the tacrolimus dose is adjusted to maintain a whole blood 12-hour trough of 4-10 ng/ml, 5-9 ng/ml, or 6-8 ng/mL. In some embodiments, 5.5-6.5 months after the subject was administered the composition comprising a population of stem cell-derived islet cells, and if the subject is not also being administered sirolimus, the tacrolimus dose is adjusted to maintain a whole blood 12-hour trough of 6-8 ng/mL.

In some aspects, the disclosure is directed to a method of treating a subject having diabetes, comprising treating the subject with: a) mycophenolate mofetil or mycophenolate sodium (e.g., 100-5000 mg of mycophenolate mofetil or mycophenolate sodium) and b) a composition comprising a population of cells, wherein the population comprises a plurality of cells that express NKX6.1 and ISL1. In some embodiments, the subject is treated with 100-5000 mg of mycophenolate mofetil or mycophenolate sodium. In some embodiments, the subject is treated with 100-3000 mg of mycophenolate mofetil or mycophenolate sodium. In some embodiments, the subject is treated with 100-2000 mg of mycophenolate mofetil or mycophenolate sodium. In some embodiments, the subject is treated with 100-1000 mg of mycophenolate mofetil or mycophenolate sodium. In some embodiments, the subject is treated with 300-5000 mg of mycophenolate mofetil or mycophenolate sodium. In some embodiments, the subject is treated with 300-2000 mg of mycophenolate mofetil or mycophenolate sodium. In some embodiments, the subject is treated with 300-1000 mg of mycophenolate mofetil or mycophenolate sodium. In some embodiments, the subject is treated with 500-1000 mg of mycophenolate mofetil or mycophenolate sodium. In some embodiments, the subject is treated with 700-1200 mg of mycophenolate mofetil or mycophenolate sodium. In some embodiments, the subject is treated with 800-1200 mg of mycophenolate mofetil or mycophenolate sodium. In some embodiments, the subject is treated with 400-900 mg of mycophenolate mofetil or mycophenolate sodium. In some embodiments, the subject is treated with 400-700 mg of mycophenolate mofetil or mycophenolate sodium. In some embodiments, the mycophenolate mofetil or mycophenolate sodium is administered daily. In some embodiments, the mycophenolate mofetil or mycophenolate sodium is administered orally. In some embodiments, the subject is further administered antimicrobial prophylaxis.

### Antimicrobial Prophylaxis

In certain aspects, the disclosure is directed to a method of treating a subject, such as the subject having diabetes who received an organ transplant, comprising treating the subject with a) an antimicrobial agent; and b) a composition comprising a population of cells, wherein the population comprises a plurality of cells that express NKX6.1 and ISL1. In some embodiments, the antimicrobial agent is administered as part of an antimicrobial prophylaxis regimen. As used herein, “antimicrobial prophylaxis” is administration of one or more antimicrobial agents for the prevention of infectious diseases. In some embodiments, the antimicrobial agent is trimethoprim, sulfamethoxazole, clotrimazole, valganciclovir, or any combination thereof. In particular embodiments, the subject is administered trimethoprim and/or sulfamethoxazole. In some embodiments, the trimethoprim/sulfamethoxazole administration is started after Day 1 of the administration of the first pharmaceutical composition. In some embodiments, the administration of trimethoprim/sulfamethoxazole is started between Day 2 and Day 5 of the administration of the composition comprising a population of cells. In some embodiments, the administration of trimethoprim/sulfamethoxazole is started on Day 2 of the administration of the composition comprising a population of cells. In some embodiments, the trimethoprim is administered at a dose of 10-500, 10-400, 10-300, 10-200, 10-100, 50-200, 50-150, 50-100, or 65-90 mg. In some embodiments, the trimethoprim is administered at a dose of 50-100 mg. In some embodiments, the sulfamethoxazole is administered at a dose of 50-1000, 50-800, 50-600, 50-500, 50-400, 50-300, 50-200, 50-100, 250-1000, 250-800, 250-600, 250-500, 250-400, 350-1000, 350-800, 350-600, 350-500, or 350-450 mg. In some embodiments, the sulfamethoxazole is administered at a dose of 350-450 mg. In some embodiments, the trimethoprim/sulfamethoxazole administered at a dose of 80 mg/400 mg once daily (QD). In some embodiments, the trimethoprim/sulfamethoxazole administration is discontinued 6 months after the administration of the composition comprising a population of cells. In some embodiments, the clotrimazole administration is started before Day 1 of the administration of the composition comprising a population of cells. In some embodiments, the clotrimazole is started between Day −5 and Day −1 of the administration of the composition comprising a population of cells. In some embodiments, the clotrimazole is started on Day −2 of the administration of the composition comprising a population of cells. In some embodiments, the clotrimazole is administered 4 times a day. In some embodiments, the clotrimazole is discontinued 3 months after the administration of the first pharmaceutical composition. In some embodiments, the clotrimazole is replaced by the administration of an antifungal prophylaxis. In some embodiments, the valganciclovir administration is started before Day 1 of the administration of the composition comprising a population of cells. In some embodiments, the valganciclovir is started between Day −5 and Day −1 of the administration of the composition comprising a population of cells. In some embodiments, the valganciclovir is started on Day −2 of the administration of the composition comprising a population of cells. In some embodiments, the valganciclovir is administered at a dose 450 mg daily (QD) and increases to 900 mg QD by Day 13. In some embodiments, the valganciclovir is administered for at least 14 weeks after the administration of the composition comprising a population of cells. In some embodiments, the subject is cytomegalovirus negative and viral prophylaxis is replaced/substituted with acyclovir. In some embodiments, acyclovir is given at a dose in a range of 50 to 1000 mg. In some embodiments, acyclovir is given at a dose in a range of 100 to 900 mg. In some embodiments, acyclovir is given at a dose in a range of 200 to 800 mg. In some embodiments, acyclovir is given at a dose in a range of 300 to 700 mg. In some embodiments, acyclovir is given at a dose in a range of 400 to 600 mg. In some embodiments, acyclovir is given at a dose in a range of 450 to 550 mg. In some embodiments, acyclovir is given at a dose of 400 mg. In some embodiments, acyclovir is given twice a day (BID).

In some embodiments, an antimicrobial (e.g., clotrimazole) will be administered (e.g., as 1 troche PO 4 times per day (qid)) beginning on Day −2 before the first SC-islet treatment (Day −1 for second or subsequent SC-islet infusion) and continuing for 3 months after the last SC-islet infusion. In some embodiments, antifungal prophylaxis per standard practice at each site may be administered instead of clotrimazole. In some embodiments, an antimicrobial (e.g., valganciclovir) will be administered (e.g., at a dose of 450 mg PO qd) beginning on Day −2 before the first SC-islet infusion (Day −1 for second or subsequent dose), increasing (e.g., to 900 mg qd) by Day 13 and continuing for at least 1, 2, 3, 4, 5, 6, 7, 8, or 9 monthsor 1-9, 1-6, 1-4, 1-2, 2-8, 2-6, 2-4, 4-8, 4-6, or 5-7 months after SC-islet infusion.

In certain aspects, the disclosure is related to a method of treating a subject having diabetes through administration of a composition comprising a population of cells, wherein prior to the administration of the composition comprising the population of cells, the subject received infusion of insulin at a level of at least about 20 U/day, 22 U/day, 24 U/day, 26 U/day, 28 U/day, 30 U/day, 32 U/day, or 34 U/day, and the subject is administered a second composition comprising a population of cells at a later point in time than the administration of the first population of cells. In some embodiments, the second composition is administered to the subject within 30 days of the first composition. In some embodiments, the second composition is administered to the subject at least 3 months, 4 months, 5 months, 6 months, 7 months, 8 months, 9 months, 10 months, 11 months, 12 months, 15 months or between 3-12 months, 3-10 months, 3-9 months, 3-7 months, 3-5 months, 5-12 months, 8-10 months, 7-12 months, 9-15 months, or 9-12 months after the subject is administered the first composition. In some embodiments, the first composition comprises 3.5×10⁸ to about 8.5×10⁸ cells and wherein the second composition comprises 3.5×10⁸ to about 8.5×10⁸ cells. In some embodiments, the first pharmaceutical composition comprises 3.5×10⁸ to about 8.5×10⁸ cells and wherein the second pharmaceutical composition comprises 3.5×10⁸ to about 4.5×10⁸ cells. In some embodiments, the method further comprises administering to the subject at least one immune response modulator before or concurrently with the administration of the second pharmaceutical composition. In some embodiments, the at least one immune response modulator administered before or concurrently with the administration of the second pharmaceutical composition is selected from the group consisting of THYMOGLOBULIN®, etanercept, basiliximab, tacrolimus, sirolimus, and/or mycophenolate mofetil. In some embodiments, the subject is administered basiliximab.

In certain aspects, the disclosure is directed to a method of treating a subject with diabetes, comprising: a) administering to the subject a first composition comprising a population of cells, b) administering to the subject a second composition comprising a population of cells, and c) administering to the subject an anti-CD25 binding moiety (e.g., basiliximab) concurrently with or after the administration of the second composition; wherein the first and the second composition each comprise a plurality of cells that express NKX6.1 and ISL1. In some embodiments, the subject is administered multiple doses of basiliximab. In some embodiments, the subject is administered basiliximab on the day the second composition is administered to the subject (Day A1). In some embodiments, a first administration of Basiliximab administration occurs on the day of administration of the second pharmaceutical (Day A1) followed by a second administration of Basiliximab between Day A2 and Day A8 of the of the administration of the second composition. In some embodiments, a first administration of Basiliximab administration occurs on the day of administration of the second pharmaceutical (Day A1) followed by a second administration of Basiliximab between Day A3 and Day A7 of the of the administration of the second composition. In some embodiments, a first administration of Basiliximab administration occurs on the day of administration of the second pharmaceutical (Day A1) followed by a second administration of Basiliximab between Day A4 and Day A6 of the of the administration of the second composition. In some embodiments, the second administration of Basiliximab occurs on Day A5 of the of the administration of the second pharmaceutical. In some embodiments, the Basiliximab is administered at a dose ranging from 5 mg to 50 mg. In some embodiments, the Basiliximab is administered at a dose ranging from 10 mg to 45 mg. In some embodiments, the Basiliximab is administered at a dose ranging from 15 mg to 40 mg. In some embodiments, the Basiliximab is administered at a dose ranging from 20 mg to 30 mg. In some embodiments, the Basiliximab is administered at a dose of 20 mg. In some embodiments, the Basiliximab is administered intravenously.

Certain aspects of the disclosure are directed to a method of treating a subject with diabetes, comprising: a) administering to the subject a first composition comprising a population of cells; b) administering to the subject a second composition comprising a population of cells; and c) administering to the subject a vasoactive agent (e.g., pentoxifylline) and/or one or more antimicrobial agents concurrently with, several days before, or after the administration of the second composition; wherein the first and the second composition each comprise a plurality of cells that express NKX6.1 and ISL1. In some embodiments, the subject is administered pentoxifylline. In some embodiments, the pentoxifylline administration is started before Day 1 of the administration of the second composition. In some embodiments, the pentoxifylline administration is started on Day −5 to Day −1 of the administration of the second composition. In some embodiments, the pentoxifylline administration is started on Day −2 of the administration of the second composition. In some embodiments, the pentoxifylline is administered at a dose between 250 mg and 500 mg. In some embodiments, the pentoxifylline is administered at a dose of 400 mg. In some embodiments, the pentoxifylline is administered three times a day (TID). In some embodiments, the pentoxifylline is administered through at least Day 3 following the administration of the second composition. In some embodiments, the pentoxifylline is administered through at least Day 8 following the administration of the second composition. In some embodiments, the antimicrobial agent is administered concurrently with, several days before, or after the administration of the second composition. In some embodiments, the antimicrobial agent comprises trimethoprim/sulfamethoxazole, clotrimazole and valganciclovir.

### Further Methods of Treatment

As used herein, the term “treating” and “treatment” can refer to administering to a subject, such as the subject having diabetes who previously received an organ transplant, an effective amount of a composition (e.g., cell clusters or a portion thereof) so that the subject has a reduction in at least one symptom of the disease or an improvement in the disease, for example, beneficial or desired clinical results. In some embodiments, the subject become insulin-independent following treatment. For purposes of this disclosure, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (e.g., partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis.

By “treatment,” “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

Unless otherwise explicit from the text, the terms “cell composition”, “composition comprising a population of cells” and the like are used interchangeably herein. Any of the cells or populations of cells (which also may be referred to herein as “cell populations”) disclosed herein may be in a composition, such as a pharmaceutical composition. Unless clear from the context otherwise, references to administering a subject a population of cells or cell population means the population is in a pharmaceutically acceptable composition.

A goal of diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patient, depending on other factors, such as how often the patient has low or high blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to diabetes, such as severe hypoglycemic events, diseases of the eye, kidney disease, or nerve disease.

The methods of treatment disclosed herein can result in amelioration of one or more physiological parameters related to blood glucose regulation. In some cases, the methods disclosed herein can result in at least partial restoration of glucose-responsive insulin secretion in the subject. Without wishing to be bound by a certain theory, glucose-responsive insulin secretion in the subject restored by the methods and compositions disclosed herein can lead to amelioration of one or more symptoms or complications associated with diabetes in the subject. In some aspects, the disclosure relates to a method comprising administering to a subject a pharmaceutical composition comprising a population of cells provided herein (e.g., SC-islet cells), wherein the administered cells release insulin in an amount sufficient for a reduction of blood glucose levels in the subject.

In some cases, oral glucose tolerance test or mixed meal tolerance test is performed in the morning following an overnight fast of >8 hours. The subject may be limited on strenuous exercise, alcohol, caffeine and tobacco use, all of which may influence insulin sensitivity. Mixed meal tolerance test can be conducted by following a protocol provided with a standardized meal of specified macronutrient content or a proprietary meal substitute, e.g., ENSURE® (Abbott) or BOOST® Complete Nutritional Drink High Protein (Nestlé Health Science). For oral glucose tolerance test, a 75 g anhydrous glucose dissolved in 250 ml water may be used as a reference method.

In some cases, the method elevates blood C-peptide level of the subject under fasting condition to at least 100 μmol/L, 120 μmol/L, 130 μmol/L, 150 μmol/L, 160 μmol/L, 180 μmol/L, 200 pmol/L, 210 μmol/L, 220 μmol/L, 230 μmol/L, 240 μmol/L, 250 μmol/L, 260 μmol/L, 270 μmol/L, or 280 μmol/L when measured at least about 1, 2, 3, 4, 5, or 6 months after the administration. In some embodiments, the method elevates blood C-peptide level of the subject under fasting condition to at least 100 μmol/L, 120 μmol/L, 130 μmol/L, 150 μmol/L, 160 μmol/L, 180 μmol/L, 200 μmol/L, 210 pmol/L, 220 μmol/L, 230 μmol/L, 240 μmol/L, 250 μmol/L, 260 μmol/L, 270 μmol/L, or 280 μmol/L when measured about 1, 2, 3, 4, 5, 6, 7, 8, 9, 10, 11, or 12 months after the administration. In some cases, the method elevates blood C-peptide level of the subject under fasting condition to at least 100 pmol/L, 120 μmol/L, 130 μmol/L, 150 μmol/L, 160 μmol/L, 180 μmol/L, 200 μmol/L, 210 μmol/L, 220 μmol/L, 230 μmol/L, 240 μmol/L, 250 μmol/L, 260 μmol/L, 270 μmol/L, or 280 μmol/L when measured about 3 months after the administration. In some cases, the method elevates blood C-peptide level of the subject under fasting condition to at least 100 μmol/L, 120 μmol/L, 130 μmol/L, 150 μmol/L, 160 μmol/L, 180 μmol/L, 200 μmol/L, 210 μmol/L, 220 μmol/L, 230 μmol/L, 240 pmol/L, 250 μmol/L, 260 μmol/L, 270 μmol/L, or 280 μmol/L when measured about 5 months after the administration. In some cases, the method elevates blood C-peptide level of the subject under fasting condition to about 200 μmol/L, 220 μmol/L, 240 μmol/L, 260 μmol/L, or 280 μmol/L, when measured about 3 months after the administration. In some cases, the method elevates blood C-peptide level of the subject under fasting condition to about 200 μmol/L, 220 μmol/L, 240 μmol/L, 260 μmol/L, or 280 μmol/L, when measured about 5 months after the administration. In some cases, administration of a pharmaceutical composition according to the present disclosure reduces hemoglobin A1c level of the subject. For instance, the subject's Hb1Ac level can be reduced to less than 8%, 7.5%, 7.0%, 6.5%, 6.0%, 5.5% or between 5-8%, 5-7.5%, 5-6.5%, 5-5.5%, 5.5-8%, 5.5-7%, 5.5-6.5%, 6-8%, 6-7%, or 6-6.5%, when measured at least about 1, 2, 3, 4, 5, or 6 months after the administration. In some cases, the subject's Hb1Ac level is reduced to less than 8%, 7.5%, 7.0%, 6.5%, 6.0%, 5.5% or 5% or between 5-8%, 5-7.5%, 5-6.5%, 5-5.5%, 5.5-8%, 5.5-7%, 5.5-6.5%, 6-8%, 6-7%, or 6-6.5%, when measured about 1, 2, 3, 4, 5, 6, 7, 8, 9, 10, 11, or 12 months after the administration. In some cases, the treated subject's Hb1Ac level is 7-7.9% when measured about 1, 2, 3, 4, 5, 6, 7, 8, 9, 10, 11, or 12 months after the administration. In some cases, it is reduced to less than 8%, 7.5%, 7.0%, or 6.75% or between 5-8.0%, 6.5-8.0%, 6.5-7.5%, 6.5-7.0%, or 7-8%, when measured about 3 months after the administration. In some cases, it is reduced to less than 8%, 7.9%, 7.8%, 7.7%, 7.6%, 7.5%, 7.3%, 7.2%, 7.1%, or 7.0%, when measured about 5 months after the administration. In some cases, it is reduced to less than 8%, 7.5%, 7.0%, 6.5%, 6.0%, 5.5%, or 5%, or between 4.5-8%, 5-8%, 5-7.5%, 5-7%, 5-6.5%, 5-5.5%, 5.5-8%, 5.5-7%, 5.5-6.5%, 6-8%, 6-7%, or 6-6.5%, when measured about 9 months after the administration. In some cases, it is reduced to about 7.6%, 7.5%, 7.4%, 7.3%, 7.2%, 7.1%, or 7.0%, when measured about 5 months after the administration.

Patients with a disease characterized by high blood glucose levels, e.g., diabetes, may have a medical need to receive infusion of external insulin to manage blood glucose level (“glycemic control”). Typically, external insulin is administered to such patients with a predetermined goal for glycemic control, e.g., to maintain blood glucose level within a certain range, such as those discussed in Louis Monnier and Claude Colette, Target for Glycemic Control, Diabetes Care November 2009, 32 (suppl 2) S199-S204; DOI: 10.2337/dc09-S310, which is incorporated herein by its entirety. In some cases, the subject may be prescribed by a medical doctor with a personalized glycemic control goal to guide his/her insulin intake. The method disclosed herein can reduce the subject's reliance on intake of external insulin while realizing his/her goal for glycemic control. In some cases, at least about 1, 2, 3, 4, 5, or 6 months after the administration, the subject's daily insulin is reduced to at most 10 units (U), 9 U, 8 U, 7 U, 6 U, 5 U, 4 U, 3 U, or 2 U in average over a 2-day, 3-day, 4-day, 5-day, 6-day, 7-day, 8-day, 9-day, or 10-day period, or over a period of at least 10 days, 15 days, 20 days, or a month. In some cases, at least about 1, 2, 3, 4, 5, or 6 months after the administration, the subject's daily insulin is reduced to about 10 units (U), 9 U, 8 U, 7 U, 6 U, 5 U, 4 U, 3 U, 2 U, or 1 U in average over a 2-day, 3-day, 4-day, 5-day, 6-day, 7-day, 8-day, 9-day, or 10-day period, or over a period of at least 10 days, 15 days, 20 days, or a month. In some cases, about 1, 2, 3, 4, 5, 6, 7, 8, 9, 10, 11, or 12 months after the administration, the subject does not need to take insulin infusion over a 2-day, 3-day, 4-day, 5-day, 6-day, 7-day, 8-day, 9-day, or 10-day period, or over a period of at least 10 days, 15 days, 20 days, or a month.

In some embodiments, the reduction of blood glucose levels in the subject, as induced by administration of the pharmaceutical composition provided herein, results in an amount of glucose which is lower than the diabetes threshold. In some embodiments, the subject is a mammalian subject. In some embodiments, the mammalian subject is human. In some embodiments, the amount of glucose is reduced to lower than the diabetes threshold in 1, 2, 3, 4, 5, 6, 7, 8, 9, or 10 days after the administration.

A subject that can be treated by the methods herein can be a human or a non-human animal. In some cases, a subject can be a mammal. Examples of a subject include but are not limited to primates, e.g., a monkey, a chimpanzee, a baboon, or a human. In some cases, a subject is a human. A subject can be non-primate animals, including, but not limited to, a dog, a cat, a horse, a cow, a pig, a sheep, a goat, a rabbit, and the like. In some cases, a subject receiving the treatment is a subject in need thereof, e.g., a human in need thereof.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having diabetes (e.g., Type 1 or Type 2), one or more complications related to diabetes, or a pre-diabetic condition, and optionally, but need not have already undergone treatment for the Diabetes, the one or more complications related to diabetes, or the pre-diabetic condition. A subject can also be one who is not suffering from diabetes or a pre-diabetic condition. A subject can also be one who has been diagnosed with or identified as suffering from diabetes, one or more complications related to diabetes, or a pre-diabetic condition, but who show improvements in known diabetes risk factors as a result of receiving one or more treatments for diabetes, one or more complications related to diabetes, or the pre-diabetic condition. Alternatively, a subject can also be one who has not been previously diagnosed as having diabetes, one or more complications related to diabetes, or a pre-diabetic condition. For example, a subject can be one who exhibits one or more risk factors for diabetes, complications related to diabetes, or a pre-diabetic condition, or a subject who does not exhibit Diabetes risk factors, or a subject who is asymptomatic for diabetes, one or more diabetes-related complications, or a pre-diabetic condition. A subject can also be one who is suffering from or at risk of developing diabetes or a pre-diabetic condition. A subject can also be one who has been diagnosed with or identified as having one or more complications related to diabetes or a pre-diabetic condition as defined herein, or alternatively, a subject can be one who has not been previously diagnosed with or identified as having one or more complications related to diabetes or a pre-diabetic condition.

In some embodiments, prior to administration of any of the pharmaceutical compositions disclosed herein, the subject has stimulated blood C-peptide level of less than 100 μmol/L, when measured using a mixed meal tolerance test, such as less than 80 μmol/L, 60 μmol/L, 40 μmol/L, 30 pmol/L, 20 μmol/L, or 10 μmol/L. In some embodiments, prior to administration of the pharmaceutical composition disclosed herein, the subject has stimulated blood C-peptide level of less than 100 μmol/L, when measured using an oral glucose tolerance test, such as less than 80 μmol/L, 60 pmol/L, 40 μmol/L, 30 μmol/L, 20 μmol/L, or 10 μmol/L. In some cases, prior to the administration, the subject has undetectable stimulated blood C-peptide level, e.g., when measured using a mixed meal tolerance test or using oral glucose tolerance test. In some cases, prior to the administration, the subject has undetectable stimulated blood C-peptide level when measured under fasting condition. In some embodiments, prior to the administration, the subject presented with no residual endogenous islet cell function. In some embodiments, the no residual endogenous islet cell function is indicated by undetectable fasting C-peptide and undetectable stimulated C-peptide at the Mixed Meal Tolerance Test. In some embodiments, the no residual endogenous islet cell function is indicated by sustained stimulated glucose levels greater than 350, 400, 450, 475, or 500 mg/dL. In some embodiments, prior to the administration, the subject was receiving greater than 10, 15, 20, 25, 30 or 35 units per day of insulin. In some cases, prior to the administration, the subject has a baseline plasma level of hemoglobin A1c of more than 8.0%, such as more than 8.1%, 8.2%, 8.3%, 8.4%, 8.5%, 8.6%, 8.8%, or 9.0%. In some embodiments, prior to the administration, the subject has an HbA1c level greater than 7.7%, 8%, 8.2%, or 8.5%. In some cases, prior to the administration, the subject receives infusion of insulin at a level of at least about 20 U/day, such as at least about 22 U/day, 24 U/day, 26 U/day, 28 U/day, 30 U/day, 32 U/day, or 34 U/day. In some cases, prior to the administration, the subject has a medical history of severe hypoglycemic events, for instance, the subject may have at least 1 severe hypoglycemic event every month, such as at least 2, 3, 4, 5, 6, 7, 8, 9, 10, 12, 15, or 20 severe hypoglycemic events. “Severe hypoglycemic event” can refer to an episode in which the person with diabetes requires the assistance of another to increase blood glucose, usually by administration of glucagon or contacting a medical professional. By depriving the brain of glucose, severe hypoglycemia can acutely alter brain function, resulting in neuroglycopenic symptoms, seizures, or even death. In some cases, prior to the administration, the subject has a medical history of impaired hypoglycemia awareness.

In some embodiments, the disclosure provides for a method of administering a population of cells (e.g., a population of cells expressing NKX6.1 and ISL1) to a subject in need thereof. In some embodiments, the disclosure provides for administering to a subject a first pharmaceutical composition comprising between 1×10⁸ cells and 12×10⁸ (e.g., 8×10⁸ to 12×10⁸) SC-islet cells. In some embodiments, the disclosure provides for administering to a subject a first pharmaceutical composition comprising between 1×10⁸ cells and 12×10⁸ (e.g., 8×10⁸ to 12×10⁸) SC-islet cells, in a single administration (i.e., in a single infusion, or in a single surgical procedure implanting one or more devices into one or more incision sites in the subject). In some embodiments, the disclosure provides for administering to a subject a first pharmaceutical composition comprising between 1×10⁸ cells and 12×10⁸ (e.g., 8×10⁸ to 12×10⁸) SC-islet cells, in multiple administrations (i.e. in two or more infusions, or in multiple surgical procedures implanting one or more devices into one or more incision sites in the subject). In some embodiments, the disclosure provides for administering to a subject a first pharmaceutical composition comprising between 1×10⁸ cells and 10×10⁸ (e.g., 3×10⁸ to 8.5×10⁸) SC-islet cells. In some embodiments, the first pharmaceutical composition comprises at least about 1×10⁸ SC-islet cells, such as at least about 1.5×10⁸, at least about 2×10⁸, at least about 2.5×10⁸, at least about 3×10⁸, at least about 3.5×10⁸, at least about 4×10⁸, at least about 4.5×10⁸, at least about 5×10⁸, at least about 5.5×10⁸, at least about, at least about 6×10⁸, at least about 6.5×10⁸, or at least about 7×10⁸ SC-islet cells. In some cases, the first pharmaceutical composition disclosed herein comprises at most about 8×10⁸, at most about 7×10⁸, at most about 6.5×10⁸, at most about 6×10⁸, at most about 5.5×10⁸, at most about 5×10⁸, at most about 4.5×10⁸, at most about 4×10⁸, at most about 3.5×10⁸, at most about 3×10⁸, at most about 2.5×10⁸, or at most about 2×10⁸ SC-islet cells. In some embodiments, the first pharmaceutical composition comprises about 1×10⁸ to about 8×10⁸, about 1×10⁸ to about 7×10⁸, about 1×10⁸ to about 6×10⁸, about 1×10⁸ to about 5×10⁸, about 1×10⁸ to about 4×10⁸, about 1×10⁸ to about 3×10⁸, about 1×10⁸ to about 2×10⁸, about 1.5×10⁸ to about 6.5×10⁸, about 2×10⁸ to about 7×10⁸, about 2×10⁸ to about 6×10⁸, about 2×10⁸ to about 5×10⁸, about 2×10⁸ to about 4×10⁸, about 2×10⁸ to about 3×10⁸, about 2.5×10⁸ to about 5.5×10⁸, about 3×10⁸ to about 7×10⁸, about 3×10⁸ to about 6×10⁸, about 3×10⁸ to about 5×10⁸, about 3×10⁸ to about 4×10⁸, about 3.5×10⁸ to about 4.5×10⁸, or about 3.8×10⁸ to about 4.2×10⁸ SC-islet cells. In some cases, the first pharmaceutical composition comprises about 3.5×10⁸ to about 4.5×10⁸ SC-islet cells. In some embodiments, the first pharmaceutical composition comprises about 3.5×10⁸ to about 8.5×10⁸ SC-islet cells. In some embodiments, the first pharmaceutical composition comprises about 3.9×10⁸ to about 4.1×10⁸ SC-islet cells. In some embodiments, the first pharmaceutical composition comprises about 7.9×10⁸ to about 8.1×10⁸ SC-islet cells. In some embodiments, the first pharmaceutical composition comprises about 7.5×10⁸ to 12×10⁸ SC-islet cells (e.g., 7.5×10⁸ to 10×10⁸ SC-islet cells). In some embodiments, the subject receives only a single administration (e.g., via infusion or via one or more of any of the devices disclosed herein) of the pharmaceutical composition, wherein the composition comprises between 7×10⁸ and 14×10⁸, between 8×10⁸ and 13×10⁸, between 9×10⁸ and 12×10⁸, between 9×10⁸ and 11×10⁸, or between 9×10⁸ and 10×10⁸ SC-islet cells. In some embodiments, the subject is administered the cells in a single administration (i.e., in a single infusion, or in a single surgical procedure implanting one or more devices into one or more incision sites in the subject).

In some embodiments, the subject is administered a first pharmaceutical composition comprising SC-islet cells, and the subject is not administered a further pharmaceutical composition comprising SC-islet cells (i.e., the first pharmaceutical composition comprising SC-islet cells administered to the subject is the only pharmaceutical composition comprising SC-islet cells administered to the subject). In some embodiments, the subject is administered a first pharmaceutical composition comprising about 3.5×10⁸ to about 4.5×10⁸ SC-islet cells, about 3.9×10⁸ to about 4.1×10⁸ SC-islet cells, 7.5×10⁸ to about 8.5×10⁸ SC-islet cells, about 7.5×10⁸ to about 9×10⁸ SC-islet cells, about 7.5×10⁸ to about 10×10⁸ SC-islet cells, about 10×10⁸ to about 12×10⁸ SC-islet cells, about 7.9×10⁸ to about 8.1×10⁸ SC-islet cells, about 4.0×10⁸ SC-islet cells, or about 8.0×10⁸ SC-islet cells, and the subject is not administered a further pharmaceutical composition comprising SC-islet cells. In some embodiments, the subject is administered a first pharmaceutical composition comprising SC-islet cells, and the subject is not administered a further pharmaceutical composition comprising SC-islet cells within 3, 4, 5, 6, 7, 8, 9 or 10 years or 1-10, 1-7, 1-5, 1-3, 3-10, 3-7, 3-5, 5-10, 5-7, or 7-10 years of having received the first pharmaceutical composition comprising SC-islet cells. In some embodiments, the subject is administered a first pharmaceutical composition comprising SC-islet cells, and the subject is not administered a further pharmaceutical composition comprising SC-islet cells within 5 years of having received the first pharmaceutical composition comprising SC-islet cells. In some embodiments, the subject is administered a first pharmaceutical composition comprising SC-islet cells, and the subject is not administered a further pharmaceutical composition comprising SC-islet cells within 10 years of having received the first pharmaceutical composition comprising SC-islet cells.

In some embodiments, the subject is administered a first pharmaceutical composition comprising SC-islet cells, and the subject is not administered a further pharmaceutical composition comprising SC-islet cells. In some embodiments, the subject is administered one or more pharmaceutical compositions comprising SC-islet cells in a single surgical procedure, and the subject is not administered any further pharmaceutical composition comprising SC-islet cells. In some embodiments, the subject is administered a first pharmaceutical composition comprising SC-islet cells, and the subject is administered at least a second pharmaceutical composition comprising SC-islet cells. In some embodiments, the subject is administered one or more pharmaceutical compositions comprising SC-islet cells in a first surgical procedure, and the subject is further administered one or more pharmaceutical compositions comprising SC-islet cells in a second surgical procedure. In some embodiments, the subject is administered a second pharmaceutical composition comprising SC-islet cells but is not administered a third pharmaceutical composition comprising SC-islet cells in a one year period. In some embodiments, the subject is administered one or more pharmaceutical compositions comprising SC-islet cells in a first surgical procedure, and the subject is further administered one or more pharmaceutical compositions comprising SC-islet cells in a second surgical procedure, but the subject is not further administered any pharmaceutical compositions comprising SC-islet cells in a one year period. In some embodiments, the subject is administered a first pharmaceutical composition comprising about 3.5×10⁸ to about 4.5×10⁸ SC-islet cells, about 3.9×10⁸ to about 4.1×10⁸ SC-islet cells, or about 4.0×10⁸ SC-islet cells and the subject is administered at a later point in time a second pharmaceutical composition comprising about 3.5×10⁸ to about 4.5×10⁸ SC-islet cells, about 3.9×10⁸ to about 4.1×10⁸ SC-islet cells, or about 4.0×10⁸ SC-islet cells. In some embodiments, the second pharmaceutical composition is administered to the subject at least 3 months, 4 months, 5 months, 6 months, 7 months, 8 months, 9 months, 10 months, 11 months, 12 months, or 15 months or between 3-12 months, 3-10 months, 3-9 months, 3-7 months, 3-5 months, 5-12 months, 8-10 months, 7-12 months, 9-15 months, or 9-12 months after the subject is administered the first pharmaceutical composition.

In some embodiments, the subject is administered a first pharmaceutical composition comprising about 3.5×10⁸ to about 8.5×10⁸ SC-islet cells, about 3.5×10⁸ to about 4.5×10⁸ SC-islet cells, about 3.9×10⁸ to about 4.1×10⁸ SC-islet cells, 7.5×10⁸ to about 8.5×10⁸ SC-islet cells, about 7.9×10⁸ to about 8.1×10⁸ SC-islet cells, about 4.0×10⁸ SC-islet cells, or about 8.0×10⁸ SC-islet cells, and the subject is administered a second pharmaceutical composition comprising about 3.5×10⁸ to about 8.5×10⁸ SC-islet cells, about 3.5×10⁸ to about 4.5×10⁸ SC-islet cells, about 3.9×10⁸ to about 4.1×10⁸ SC-islet cells, 7.5×10⁸ to about 8.5×10⁸ SC-islet cells, about 7.9×10⁸ to about 8.1×10⁸ SC-islet cells, about 4.0×10⁸ SC-islet cells, or about 8.0×10⁸ SC-islet cells. In some embodiments, the second pharmaceutical composition is administered at least 3, 4, 5, 6, 7, 8, 9 or 10 years or between 3-20, 10-20, 15-20, 5-15, 3-5, 5-8, 8-10, 10-12, 12-15, or 20-25 years after the first pharmaceutical composition is administered to the subject.

### Pharmaceutical Compositions

In some embodiments described herein, the method administers a pharmaceutical composition, e.g., on “Day 1.” In some embodiments, the method administers a second pharmaceutical composition at some time point after Day 1. In embodiments where a second pharmaceutical composition is administered, the second pharmaceutical composition is said to be administered on “Day A1.” The compositions can be administered to the subject having diabetes who received an organ transplant.

In some embodiments, the pharmaceutical composition used in the methods disclosed herein comprises from about 1×10⁸ cells to about 12×10⁸ (e.g., 3×10⁸ to 8.5×10⁸) cells. In some embodiments, the pharmaceutical composition comprises at least about 1×10⁸ cells, such as at least about 1.5×10⁸, at least about 2×10⁸, at least about 2.5×10⁸, at least about 3×10⁸, at least about 3.5×10⁸, at least about 4×10⁸, at least about 4.5×10⁸, at least about 5×10⁸, at least about 5.5×10⁸, at least about, at least about 6×10⁸, at least about 6.5×10⁸, or at least about 7×10⁸ cells the population of cells. In some cases, the pharmaceutical composition disclosed herein comprises at most about 8×10⁸, at most about 7×10⁸, at most about 6.5×10⁸, at most about 6×10⁸, at most about 5.5×10⁸, at most about 5×10⁸, at most about 4.5×10⁸, at most about 4×10⁸, at most about 3.5×10⁸, at most about 3×10⁸, at most about 2.5×10⁸, or at most about 2×10⁸ cells in the population of cells. In some embodiments, the population of cells in the pharmaceutical composition comprises about 1×10⁸ to about 8×10⁸, about 1×10⁸ to about 7×10⁸, about 1×10⁸ to about 6×10⁸, about 1×10⁸ to about 5×10⁸, about 1×10⁸ to about 4×10⁸, about 1×10⁸ to about 3×10⁸, about 1×10⁸ to about 2×10⁸, about 1.5×10⁸ to about 6.5×10⁸, about 2×10⁸ to about 7×10⁸, about 2×10⁸ to about 6×10⁸, about 2×10⁸ to about 5×10⁸, about 2×10⁸ to about 4×10⁸, about 2×10⁸ to about 3×10⁸, about 2.5×10⁸ to about 5.5×10⁸, about 3×10⁸ to about 7×10⁸, about 3×10⁸ to about 6×10⁸, about 3×10⁸ to about 5×10⁸, about 3×10⁸ to about 4×10⁸, about 3.5×10⁸ to about 4.5×10⁸, about 3.8×10⁸ to about 4.2×10⁸ cells, about 7.5×10⁸ to about 10×10⁸ cells, or about 8×10⁸ to about 12×10⁸ cells. In some cases, the population of cells in the pharmaceutical composition comprises about 3.5×10⁸ to about 4.5×10⁸ cells. In some embodiments, the population of cells in the pharmaceutical composition comprises about 3.5×10⁸ to about 8.5×10⁸ cells. In some embodiments, the population of cells in the pharmaceutical composition comprises about 3.9×10⁸ to about 4.1×10⁸ cells. In some embodiments, the population of cells in the pharmaceutical composition comprises about 7.9×10⁸ to about 8.1×10⁸ cells.

In some embodiments, to formulate the dose, a cell cluster suspension is first counted as dissociated single cells using an optical method that detects individual cells by phase contrast. After cells are counted in the suspension, the pelleted stock of cells may be adjusted to the appropriate volume and formulated in a solution (e.g., hyopthermasol) to a desired concentration (e.g., 2 million cells per milliliter for a total volume). For example, if the target dose is 430e6 cells, 215 mL of hypothermasol may be added.

In one embodiment, to achieve a targeted dose, two 0.5 mL samples are taken from the pre-formulated harvested drug substance for cell count and viability. Gross weight of the pooled cell suspension may be taken of pre-formulated drug substance once in the infusion medium and Total volume of Cell suspension is calculated with the Tared Tube starting mass. In order for material release, cells may be subject to cell count and viability via hemocytometer. Trypan exclusion method, in tandem with a disposable Neubauer hemocytometer, may be utilized to assess cell density of the pooled drug substance suspension. This method may be qualified to accurately count cells in a single cell suspension within concentration ranges of 0.4e6 to 2.5e6. Dose may be assessed with a manual cell counting process that relies on the exclusion method (Trypan Blue) post TrypLE enzymatic dissociation. Upon staining, live cells are impermeable to Trypan Blue and retain a white appearance, where dead cells are permeable to the dye and turn blue. This method provides reportable values for cell number (total cells/mL or viable cells/mL) and % viability of the pooled cell suspension. In some embodiments, cell count is assessed with two biological replicates, with three technical replicates per biological replicate and two operators executing the count. Final cell concentration may be determined with the following: cells/mL=average manual count per quadrant×final dilution factor×10e4. In some embodiments, for each count to be valid, each technical replicate ranges from 80-500 cells, and is also within 35% of the mean values of the technical replicates counted for each biological replicate. In some embodiments, the % difference between biological replicates must be less than or equal to 25% to calculate a reportable result for cell concentration and viability. In some embodiments, cell concentration (Viable Cells/mL) may be reported, and Viable Cells may be calculated by multiplying the Volume measured in the Gross Weight assessment of the pooled cell suspension by the cell concentration disclosed herein.

Total viable cells =Volume of pooled suspension of Drug Substance×Viable Cell Concentration

### Cell Compositions

In some embodiments, the pharmaceutical composition used in the methods disclosed herein comprises a plurality of cells, e.g., any of the cell populations disclosed herein. In some embodiments, the cells are SC-β cells. The SC-β cells of the disclosure share many characteristic features of B cells which are important for normal β cell function. In some embodiments, the SC-β cell exhibits a glucose stimulated insulin secretion (GSIS) response in vitro. In some embodiments, the SC-β cell exhibits a GSIS response in vivo. In some embodiments, the SC-β cell exhibits in vitro and in vivo GSIS responses. In some embodiments, the GSIS responses resemble the GSIS responses of an endogenous mature pancreatic β cell. In some embodiments, the SC-cell exhibits a GSIS response to at least one glucose challenge. In some embodiments, the SC-β cell exhibits a GSIS response to at least two sequential glucose challenges. In some embodiments, the SC-β cell exhibits a GSIS response to at least three sequential glucose challenges. In some embodiments, the GSIS responses resemble the GSIS response of endogenous human islets to multiple glucose challenges. In some embodiments, the GSIS response is observed immediately upon transplanting the cell into a human or animal. In some embodiments, the GSIS response is observed within approximately 24 hours of transplanting the cell into a human or animal. In some embodiments, the GSIS response is observed within approximately one week of transplanting the cell into a human or animal. In some embodiments, the GSIS response is observed within approximately two weeks of transplanting the cell into a human or animal. In some embodiments, the stimulation index of the cell as characterized by the ratio of insulin secreted in response to high glucose concentrations compared to low glucose concentrations is similar to the stimulation index of an endogenous mature pancreatic β cell. In some embodiments, the SC-β cell exhibits a stimulation index of greater than 1. In some embodiments, the SC-β cell exhibits a stimulation index of greater than or equal to 1. In some embodiments, the SC-β cell exhibits a stimulation index of greater than 1.1. In some embodiments, the SC-cell exhibits a stimulation index of greater than or equal to 1.1. In some embodiments, the SC-cell exhibits a stimulation index of greater than 2. In some embodiments, the SC-β cell exhibits a stimulation index of greater than or equal to 1. In some embodiments, the SC-β cell exhibits a stimulation index of at least 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or greater.

In particular embodiments, the SC-β cells and SC-islet cells disclosed herein are generated in vitro. Examples of protocols for generating any of the SC-β cells/islets disclosed herein are described in U.S. Pat. Nos. 10,030,229, 10,443,042, and 11,466,256; published application US 20200332262, US20230218676, WO2023077140, US20210198632, and US20220090020, and published application WO2022147056, each of which is incorporated by reference in its entirety. In some embodiments, the SC-β cells and/or SC-islet cells are those purportedly described in any of Sakuma et al. Stem Cell Research & Therapy (2023) 14:1; Wang et al., 2024, Cell, 187:1-13; Wu et al., 2024, Cell Discovery, 10 (45); WO2022043518; WO2024008810; Hogrebe et al., Nat Protoc. 2021 Sep. 16 (9): 4109-4143; WO2023203208; and/or Ratiu et al., 2023, bioRxiv, doi.org/10.1101/2023.10.20.563345. In some embodiments, the disclosure provides for a composition comprising any of the cell populations described in any of WO2012175633; WO2015028614; WO2017144695; WO2019048690; WO2020207998; WO2022043518; WO2023203208; WO2024008810; WO2025029826; WO2025093467; Wang et al., 2024, Cell, 187:1-13; Wu et al., 2024, Cell Discovery, 10 (45); WO2025117331; U.S. Pat. No. 11,274,280; Hogrebe et al., Nat Protoc., 2021, 16 (9): 4109-4143; Vegas et al., 2016, Nat Med. 2016 March; 22 (3): 306-311; Wu et al., 2024, Cell Discovery, 10 (45); and Ratiu et al., 2023, bioRxiv, https://doi.org/10.1101/2023.10.20.563345, each of which is herein incorporated by reference in its entirety.

In some embodiments the SC-β cells and/or SC-islet cells disclosed herein are generated by culturing in a medium comprising a water-soluble synthetic polymer. In some embodiments, the water-soluble synthetic polymer is polyvinyl alcohol, poloxamer, polyvinylpyrrolidone, polyethylene glycol (PEG), PEG copolymers, poly(N-isopropylacrylamide), or polyacrylamide. In some embodiments, the water-soluble synthetic polymer is polyvinyl alcohol (PVA). In some embodiments, the water-soluble synthetic polymer has a concentration of 0.005% to 0.5% (w/v), 0.01% to 0.2% (w/v), 0.02% to 0.1% (w/v), or 0.03% to 0.08% (w/v) of the medium. In some embodiments, the PVA is at most 90% hydrolyzed, optionally wherein the PVA is about 87%-89% hydrolyzed. In some embodiments the PVA is about 80% hydrolyzed.

In preferred embodiments, the SC-β cells express both NKX6.1 and ISL1. In some cases, the cell compositions in the pharmaceutical composition used in the methods disclosed herein have at least about 30% cells expressing C-peptide and not expressing VMAT1, as measure by flow cytometry. In some cases, the cell composition in the pharmaceutical composition used in the methods disclosed herein has at least about 35% cells expressing C-peptide and not expressing VMAT1, as measure by flow cytometry. In some cases, the expression of C-peptide and absence of VMAT1 in a cell of the cell compositions suggest that the cell is a SC-β cell. In some cases, the cell composition has at least about 30%, 32%, 33%, 34%, 35%, 36%, 37%, 38%, 39%, 40%, 41%, 42%, 43%, 44%, 45%, 46%, 47%, 48%, 49%, or 50% cells expressing C-peptide and not expressing VMAT1, as measure by flow cytometry. In some cases, the cell composition has about 30%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% cells expressing C-peptide and not expressing VMAT1, as measure by flow cytometry. In some cases, the cell composition has about 30% to about 60%, about 35% to about 55%, about 40% to about 50% cells expressing C-peptide and not expressing VMAT1, as measure by flow cytometry.

In some cases, the cell composition in the pharmaceutical composition disclosed herein has at most about 35% cells expressing VMAT1, as measure by flow cytometry. In some cases, the cell composition in the pharmaceutical composition used in the methods disclosed herein has at most about 35% cells expressing VMAT1 and not expressing C-peptide, as measure by flow cytometry. In some cases, the expression of VMAT1 and absence of C-peptide in a cell of the cell compositions suggest that the cell is a SC-EC cell. In some cases, the cell composition has at most about 45%, 40%, 35%, 32%, 31%, 30%, 28%, 25%, 24%, 23%, 22%, 21%, or 20% cells expressing VMAT1 and not expressing C-peptide, as measure by flow cytometry. In some cases, the cell composition has at most about 35%, 32%, 31%, 30%, 28%, 25%, 24%, 23%, 22%, 21%, or 20% cells expressing VMAT1 and not expressing C-peptide, as measure by flow cytometry. In some cases, the cell composition has about 35%, 32%, 31%, 30%, 28%, 25%, 24%, 23%, 22%, 21%, 20%, 19%, 18%, 17%, 16%, or 15% cells expressing VMAT1 and not expressing C-peptide, as measure by flow cytometry. In some cases, the cell composition has about 15% to about 30%, about 16% to 25%, about 17% to about 22%, about 18% to about 20% cells expressing VMAT1 and not expressing C-peptide, as measure by flow cytometry. In some cases, the cell composition has 5-25%, 5-15%, 10-25%, 10-15%, or 15-25% cells expressing VMAT1 and not expressing C-peptide, as measure by flow cytometry.

In some cases, the cell composition includes at least about 3% cells expressing glucagon, as measured by flow cytometry. In some cases, the cell composition includes at least about 5% cells expressing glucagon, as measured by flow cytometry. In some cases, the cell composition includes at least about 10% cells expressing glucagon, as measured by flow cytometry. In some cases, the cell composition includes at least about 20% cells expressing glucagon, as measured by flow cytometry. In some cases, the cell composition includes at least about 15% cells expressing glucagon and not expressing somatostatin, as measured by flow cytometry. In some cases, the expression of glucagon and not expressing somatostatin in a cell of the cell composition suggest that the cell is a SC-α cell. In some cases, the cell composition includes at least about 3%, 5%, 6%, 7%, 8%, 9%, 10%, 11%, 12%, 13%, 14%, 15%, 16%, 17%, 18%, 19%, 20%, 21%, or 22% cells expressing glucagon and not expressing somatostatin, as measured by flow cytometry. In some cases, the cell composition includes at least about 12%, 13%, 14%, 15%, 16%, 17%, 18%, 19%, 20%, 21%, or 22% cells expressing glucagon and not expressing somatostatin, as measured by flow cytometry. In some cases, the cell composition includes about 10% to about 30%, about 12% to about 25%, about 13% to about 22%, about 15% to about 20%, or about 16% to about 18% cells expressing glucagon and not expressing somatostatin, as measured by flow cytometry. In some cases, the cell composition includes about 3%, 5%, 6%, 7%, 8%, 9%, 10%, 12%, 13%, 14%, 15%, 16%, 17%, 18%, 19%, 20%, 21%, or 22% cells expressing glucagon and not expressing somatostatin, as measured by flow cytometry.

In some cases, the cell composition includes at least about 1% cells expressing somatostatin and not expressing glucagon, as measured by flow cytometry. In some cases, the cell composition includes at least about 2% cells expressing somatostatin and not expressing glucagon, as measured by flow cytometry. In some cases, the cell composition includes at least about 3% cells expressing somatostatin and not expressing glucagon, as measured by flow cytometry. In some cases, the cell composition includes at least about 4% cells expressing somatostatin and not expressing glucagon, as measured by flow cytometry. In some cases, the expression of glucagon and not expressing somatostatin in a cell of the cell composition suggest that the cell is a SC-δ cell. In some cases, the cell composition includes at least about 2%, 3%, 4%, 5%, 6%, 7%, or 8% cells expressing somatostatin and not expressing glucagon, as measured by flow cytometry. In some cases, the cell composition includes about 1% to about 9%, about 2% to about 8%, about 3% to about 7%, or about 4% to about 6% cells expressing somatostatin and not expressing glucagon, as measured by flow cytometry. In some cases, the cell composition includes about 1%, 2%, 3%, 4%, 5%, 6%, 7%, or 8% cells expressing somatostatin and not expressing glucagon, as measured by flow cytometry.

In some cases, the cell composition has at least about 35% cells expressing C-peptide and not expressing VMAT1, at most about 50% cells expressing VMAT1, at least about 4% cells expressing glucagon and not expressing somatostatin, and at least about 1% cells expressing somatostatin and not expressing glucagon, as measured by flow cytometry. In some cases, the cell composition has at least about 35% cells expressing C-peptide and not expressing VMAT1, at most about 30% cells expressing VMAT1, and at least about 20% cells expressing glucagon, as measured by flow cytometry. In some cases, the cell composition has at least about 35% cells expressing C-peptide and not expressing VMAT1, at most about 30% cells expressing VMAT1, at least about 20% cells expressing glucagon, and at least 4% cells expressing somatostatin and not expressing glucagon, as measured by flow cytometry.

In some cases, the cell composition used herein includes (a) at least about 35% cells expressing C-peptide and not expressing VMAT1; and (b) at least about 10% cells expressing somatostatin, as measured by flow cytometry. In some cases, there are at least about 15% cells expressing somatostatin in the cell composition, as measured by flow cytometry.

In some cases, provided herein is a method using a composition comprising a population of cells, wherein: (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and/or (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

In some cases, provided herein is a method using a composition comprising a population of cells, wherein: (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and/or (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

In some cases, in the population of cells provided herein, 35-60% of the cells express C-peptide and ISL1 but not VMAT1; 4-25%, of the cells express glucagon but not somatostatin; and 1-10% of the cells express somatostatin but not glucagon. In some cases, less than 40%, less than 35%, less than 30%, less than 25%, less than 20%, less than 18%, less than 15%, less than 12%, or less than 10% of the cells in the population of cells provided herein express VMAT1 but not C-peptide.

In some cases, in the population of cells provided herein, 40-60% of the cells express C-peptide and ISL1 but not VMAT1; 10-25% of the cells express glucagon but not somatostatin; and 4-10% of the cells express somatostatin but not glucagon. In some cases, less than 25%, less than 20%, less than 18%, less than 15%, less than 12%, or less than 10% of the cells in the population of cells provided herein express VMAT1 but not C-peptide.

In some embodiments, any of the pharmaceutical compositions used in the methods disclosed herein comprises no less than 50%, 40%, 30%, or 20% NKX6.1⁺/ISL1⁺ cells (e.g., as determined by flow cytometry). In some embodiments, no less than 30% of the cells in the composition are NKX6.1-positive, ISL1-positive cells, no less than 25% of the cells in the composition are NKX6.1-negative, ISL1-positive cells, less than 12% of the cells in the composition are NKX6.1-negative, ISL1-negative cells or between 9-25% of the cells in the composition are NKX6.1-positive, ISL1-negative cells (e.g., as determined by flow cytometry). In some embodiments, no less than 40%, 35%, 30%, 26%, 25%, or 20% of the cells in the pharmaceutical composition are NKX6.1⁻/ISL1⁺ cells (e.g., as determined by flow cytometry). In some embodiments, no less than 26% of the cells in the pharmaceutical composition are NKX6.1⁻/ISL1⁺ cells (e.g., as determined by flow cytometry). In some embodiments, between 5-25%, 5-40%, 5-35%, or 8-20% of the cells in the pharmaceutical composition are NKX6.1⁻/ISL1⁺ cells (e.g., as determined by flow cytometry). In some embodiments, no more than 50%, 45%, 40%, 35%, 30%, or 25% of the cells in the pharmaceutical composition are NKX6.1⁺/ISL1⁻ cells (e.g., as determined by flow cytometry). In some embodiments, no more than 50% of the cells in the pharmaceutical composition are NKX6.1⁺/ISL1⁻ cells (e.g., as determined by flow cytometry).

In some embodiments, less than 12% of the cells (e.g., about 11%, about 10%, about 9%, about 8%, about 7%, about 6%, about 5%, about 4%, about 3%, about 2%, about 1%, or less) in the population are NKX6.1-negative, ISL1-negative cells. In some embodiments, less than 10%, less than 8%, less than 6%, less than 4%, or 1%-11%, 2%-10%, 2%-12%, 4%-12%, 6%-12%, 8%-12%, 2%-8%, 4%-8%, 3%-6% or 3%-5% of the cells in the population are NKX6.1-negative, ISL1-negative cells. In some embodiments, 2%-12%, 4%-12%, 6%-12%, 8%-12%, 2%-8%, 4%-8%, 3%-6% or 3%-5% of the cells in the population are NKX6.1-negative, ISL1-negative cells. In some embodiments, less than 6% or between 0-6%, 0-4%, 0-2%, 0-1%, 1-6%, 1-5%, 1-4%, 1-3%, 1-2%, 2-3%, or 3-6% of the cells in the population are NKX6.1-negative, ISL1-negative cells.

In some embodiments, at least 15% of the cells (e.g., about 15%, about 20%, about 25%, about 30%, about 35%, about 40%, about 45%, about 50%, about 55%, about 60% or more) in the population are NKX6.1-negative, ISL1-positive cells. In some embodiments, at least 15%, at least 20%, at least 25%, at least 30%, at least 35%, at least 40%, at least 45%, at least 50%, at least 55%, at least 60%, or 15%-60%, 15%-45%, 15%-30%, 30%-60%, 30%-45%, 45%-60% of the cells in the population are NKX6.1-negative, ISL1-positive cells. In some embodiments, 20%-60%, 20%-50%, 20%-40%, 20%-30%, 30%-60%, 30%-50%, 30%-40%, 40%-60%, 40%-50%, or 50%-60% of the cells in the population are NKX6.1-negative, ISL1-positive cells.

In some embodiments, at least 15% (e.g., 20%-60%, 20%-50%, 20%-40%, 20%-30%, 30%-60%, 30%-50%, 30%-40%, 40%-60%, 40%-50%, or 50%-60%) of the cells in the population are NKX6.1-negative, ISL1-positive cells and less than 12% (e.g., 0-6%, 0-4%, 0-2%, 0-1%, 1-6%, 1-5%, 1-4%, 1-3%, 1-2%, 2-3%, 3-6%, 2%-12%, 4%-12%, 6%-12%, 8%-12%, 2%-8%, 4%-8%, 3%-6% or 3%-5%) of the cells in the population are NKX6.1-negative, ISL1-negative cells.

In some embodiments, at least 60%, at least 65%, at least 70%, at least 73%, at least 74%, at least 75%, at least 80%, at least 85%, at least 90%, about 85-95%, or about 90-95% of the cells in the population are ISL1-positive cells. In some embodiments, 50-90%, 50-85%, 50-80%, 50-75%, 50-70%, 50-60%, 60-90%, 60-85%, 60-80%, 60-75%, 60-70%, 65-90%, 65-85%, 65-80%, 65-75%, 65-70%, 70-90%, 70-85%, 70-80%, 70-75%, 75-90%, 75-85%, 75-80%, 80-90%, 80-85%, or 85-90% of the cells in the population are ISL1-positive cells. In some embodiments, at least 74%, at least 75%, at least 80%, at least 85%, at least 90%, about 85-95%, about 90-95%, about 95-98%, or about 96-99% of the cells in the population are ISL1-positive cells. In some embodiments, about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% of the cells in the population are ISL1-positive cells.

In some embodiments, a population of in vitro differentiated cells described herein comprises more NKX6.1-negative, ISL1-positive cells than NKX6.1-positive, ISL1-positive cells. In some embodiments, at least 40% of the cells in the population are NKX6.1-negative, ISL1-positive cells. In some embodiments, at least 45%, at least 50%, about 40-50%, about 45-55%, or about 50-55% of the cells in the population are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the population are NKX6.1-negative, ISL1-positive cells.

In some embodiments, at least 20% (e.g., at least 20%, at least 30%, at least 40%, at least 50%, at least 50%, at least 60% or more) of the ISL1-positive cells are NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the ISL1-positive cells are NKX6.1-negative. In some embodiments, about 20%, 25%, 30%, 35%, 40%, 45%, 50%, 55%, 60% or more of the ISL1-positive cells are NKX6.1-negative.

In some embodiments, at least 20% (e.g., at least 20%, at least 30%, at least 40%, at least 50%, at least 50%, at least 60% or more) of the cells in the composition are ISL1-positive and NKX6.1-positive. In some embodiments, about 20%-60%, 20%-50%, 20%-40%, 20%-30%, 30%-60%, 30%-50%, 30%-40%, 40%-60%, 40%-50%, or 50%-60% of the cells in the composition are ISL1-positive and NKX6.1-positive. In particular embodiments, 30%-50% of the cells in the composition are ISL1-positive and NKX6.1-positive cells. In some embodiments, about 20%, 25%, 30%, 35%, 40%, 45%, 50%, 55%, 60% or more of the cells in the composition are ISL1-positive and NKX6.1-positive.

In some embodiments, at least 20% (e.g., at least 20%, at least 30%, at least 40%, at least 50%, at least 50%, at least 60% or more) of the cells in the composition are ISL1-positive and NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the cells in the composition are ISL1-positive and NKX6.1-negative. In some embodiments, about 20%, 25%, 30%, 35%, 40%, 45%, 50%, 55%, 60% or more of the cells in the composition are ISL1-positive and NKX6.1-negative.

In some embodiments, a population of in vitro differentiated cells described herein comprises up to 20% (e.g., up to 20%, up to 30%, up to 40% or up to 50%) of NXK6.1-positive, ISL1-positive cells. In some embodiments, a population of in vitro differentiated cells described herein comprises about 20%-50%, 20%-40%, 20%-30%, 30%-50%, 30%-40%, or 40%-50% of NXK6.1-positive, ISL1-positive cells. In some embodiments, a population of in vitro differentiated cells described herein comprises about 20%-50%, 20%-40%, 20%-30%, 30%-50%, 30%-40%, or 40%-50% of NXK6.1-positive, ISL1-positive cells.

In some embodiments, the NKX6.1-positive, ISL1-positive cells also express PDX1.

In some embodiments, the disclosure provides for a method using a composition comprising a plurality of cells (e.g., a composition comprising a cluster of cells or multiple clusters of cells); wherein 30-60%, 30-55%, 30-50%, 30-45%, 30-40%, 30-35%, 35-60%, 35-55%, 35-50%, 35-45%, 35-40%, 40-60%, 40-55%, 40-50%, 40-45%, 45-60%, 45-55%, 45-50%, 50-60%, or 50-55% of the cells in the composition are NKX6.1-positive, ISL1-positive cells; wherein 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, 35-50%, 35-35%, 35-40%, 40-50%, 40-45%, or 45-50% of the cells in the composition are NKX6.1-negative, ISL1-positive cells; and wherein 1-12%, 1-10%, 1-8%, 1-6%, 1-4%, 3-5%, 1-2%, 2-12%, 2-10%, 2-8%, 2-6%, 2-4%, 4-12%, 4-10%, 4-8%, 4-6%, 6-12%, 6-10%, 6-8%, 8-12%, 8-10%, or 10-12% of the cells in the composition are NKX6.1-negative, ISL1-negative cells. In some embodiments, the disclosure provides for a method using a composition comprising a plurality of cells (e.g., a composition comprising a cluster of cells or multiple clusters of cells); wherein 35-50% of the cells in the composition are NKX6.1-positive, ISL1-positive cells; wherein 30-45% of the cells in the composition are NKX6.1-negative, ISL1-positive cells; and wherein 2-12% of the cells in the composition are NKX6.1-negative, ISL1-negative cells. In some embodiments, between 0-6%, 0-4%, 0-2%, 0-1%, 1-6%, 1-5%, 1-4%, 1-3%, 1-2%, 2-3%, 3-6%, 3-25%, 3-20%, 3-15%, 3-10%, 3-5%, 5-25%, 5-20%, 5-15%, 5-10%, 10-25%, 10-20%, 10-15%, 15-25%, 15-20% or 20-25% of the cells in the composition are NKX6.1-positive, ISL1-negative cells.

In some embodiments, the disclosure provides for a method using a composition comprising a plurality of cells (e.g., a composition comprising a cluster of cells or multiple clusters of cells); wherein at least 30% of the cells in the composition are NKX6.1-positive, ISL1-positive cells; wherein at least 25% of the cells in the composition are NKX6.1-negative, ISL1-positive cells; and wherein between 9-25% of the cells in the composition are NKX6.1-positive, ISL1-negative cells. In some embodiments, the disclosure provides for a method using a composition comprising a plurality of cells (e.g., a composition comprising a cluster of cells or multiple clusters of cells); wherein 30-60%, 30-55%, 30-50%, 30-45%, 30-40%, 30-35%, 35-60%, 35-55%, 35-50%, 35-45%, 35-40%, 40-60%, 40-55%, 40-50%, 40-45%, 45-60%, 45-55%, 45-50%, 50-60%, or 50-55% of the cells in the composition are NKX6.1-positive, ISL1-positive cells; wherein 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, 35-50%, 35-35%, 35-40%, 40-50%, 40-45%, or 45-50% of the cells in the composition are NKX6.1-negative, ISL1-positive cells; and wherein 9-30%, 9-25%, 9-20%, 9-15%, 9-12%, 12-30%, 12-25%, 12-20%, 12-15%, 15-30%, 15-25%, 15-20%, 20-30%, 20-25% or 25-30% of the cells in the composition are NKX6.1-positive ISL1-negative cells. In some embodiments, 1-12%, 1-10%, 1-8%, 1-6%, 1-4%, 3-5%, 1-2%, 2-12%, 2-10%, 2-8%, 2-6%, 2-4%, 4-12%, 4-10%, 4-8%, 4-6%, 6-12%, 6-10%, 6-8%, 8-12%, 8-10%, or 10-12% of the cells in the composition are NKX6.1-negative, ISL1-negative cells. In some embodiments, the disclosure provides for a method using a composition comprising a plurality of cells (e.g., a composition comprising a cluster of cells or multiple clusters of cells); wherein 35-50% of the cells in the composition are NKX6.1-positive, ISL1-positive cells; wherein 30-45% of the cells in the composition are NKX6.1-negative, ISL1-positive cells; and wherein 9-25% of the cells in the composition are NKX6.1-positive, ISL1-negative cells.

In some embodiments, less than 12% of the cells (e.g., about 11%, about 10%, about 9%, about 8%, about 7%, about 6%, about 5%, about 4%, about 3%, about 2%, about 1%, or less) in the composition are NKX6.1-negative, ISL1-negative cells. In some embodiments, less than 10%, less than 8%, less than 6%, less than 4%, 1%-11%, 2%-10%, 2%-12%, 4%-12%, 6%-12%, 8%-12%, 2%-8%, 4%-8%, 3%-6% or 3%-5% of the cells in the composition are NKX6.1-negative, ISL1-negative cells. In some embodiments, 2%-12%, 4%-12%, 6%-12%, 8%-12%, 2%-8%, 4%-8%, 3%-6% or 3%-5% of the cells in the population are NKX6.1-negative, ISL1-negative cells.

In some embodiments, at least 15% of the cells (e.g., about 15%, about 20%, about 25%, about 30%, about 35%, about 40%, about 45%, about 50%, about 55%, about 60% or more) in the composition are NKX6.1-negative, ISL1-positive cells. In some embodiments, at least 15%, at least 20%, at least 25%, at least 30%, at least 35%, at least 40%, at least 45%, at least 50%, at least 55%, at least 60%, 15%-60%, 15%-45%, 15%-30%, 30%-60%, 30%-45%, 45%-60% of the cells in the composition are NKX6.1-negative, ISL1-positive cells. In some embodiments, 20%-60%, 20%-50%, 20%-40%, 20%-30%, 30%-60%, 30%-50%, 30%-40%, 40%-60%, 40%-50%, or 50%-60% of the cells in the composition are NKX6.1-negative, ISL1-positive cells.

In some embodiments, at least 15% (e.g., 20%-60%, 20%-50%, 20%-40%, 20%-30%, 30%-60%, 30%-50%, 30%-40%, 40%-60%, 40%-50%, or 50%-60%) of the cells in the composition are NKX6.1-negative, ISL1-positive cells and less than 12% (e.g., 2%-12%, 4%-12%, 6%-12%, 8%-12%, 2%-8%, 4%-8%, 3%-6% or 3%-5%) of the cells in the composition are NKX6.1-negative, ISL1-negative cells.

In some embodiments, at least 60%, at least 65%, at least 70%, at least 73%, at least 74%, at least 75%, at least 80%, at least 85%, at least 90%, about 85-95%, or about 90-95% of the cells in the composition are ISL1-positive cells. In some embodiments, 50-90%, 50-85%, 50-80%, 50-75%, 50-70%, 50-60%, 60-90%, 60-85%, 60-80%, 60-75%, 60-70%, 65-90%, 65-85%, 65-80%, 65-75%, 65-70%, 70-90%, 70-85%, 70-80%, 70-75%, 75-90%, 75-85%, 75-80%, 80-90%, 80-85%, or 85-90% of the cells in the composition are ISL1-positive cells. In some embodiments, at least 74%, at least 75%, at least 80%, at least 85%, at least 90%, about 85-95%, or about 90-95% of the cells in the composition are ISL1-positive cells. In some embodiments, about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% of the cells in the composition are ISL1-positive cells.

In some embodiments, the composition comprises more NKX6.1-positive, ISL1-positive cells that NKX6.1-negative, ISL1-positive cells. In some embodiments, at least 40% of the cells in the composition are NKX6.1-negative, ISL1-positive cells. In some embodiments, at least 45%, at least 50%, about 40-50%, about 45-55%, or about 50-55% of the cells in the composition are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the composition are NKX6.1-negative, ISL1-positive cells.

In some embodiments, at least 20% (e.g., at least 20%, at least 30%, at least 40%, at least 50%, at least 50%, at least 60% or more) of the ISL1-positive cells are NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the ISL1-positive cells are NKX6.1-negative. In some embodiments, about 20%, 25%, 30%, 35%, 40%, 45%, 50%, 55%, 60% or more of the ISL1-positive cells are NKX6.1-negative.

In some embodiments, the composition used in the disclosed methods comprises at least 20% (e.g., at least 20%, 30%, 40%, 50% or 60%) of NXK6.1-positive, ISL1-positive cells. In some embodiments, the composition used in the disclosed methods comprises about 20%-50%, 20%-40%, 20%-30%, 30%-50%, 30%-40%, 40%-50%, 40%-60%, or 50-60% of NXK6.1-positive, ISL1-positive cells. In some embodiments, the composition used in the disclosed methods comprises about 20%-50%, 20%-40%, 20%-30%, 30%-50%, 30%-40%, or 40%-50% of NXK6.1-positive, ISL1-positive cells.

In some embodiments, the composition used in the disclosed methods comprises less than 25% (e.g., less than 25%, less than 20%, less than 15%, less than 10%, less than 5%, or less) of NKX6.1-positive, ISL1-negative cells. In some embodiments, the composition used in the disclosed methods comprises about 2%-25%, 2%-20%, 2%-15%, 2%-10%, 2%-5%, 5%-25%, 5%-20%, 5%-15%, 5%-10%, 10%-25%, 10%-20%, 10%-15%, 15%-25%, 15%-20%, or 20%-25% of NKX6.1-positive, ISL1-negative cells. In some embodiments, the composition comprises about 2%-10%, 2%-8%, 2%-6%, 2%-4%, 4%-10%, 4%-8%, 4%-6%, 6%-10%, 6%-8%, or 8%-10% of NKX6.1-positive, ISL1-negative cells. In some embodiments, the composition used in the disclosed methods comprises about 2%, 4%, 6%, 8%, or 10% of NKX6.1-positive, ISL1-negative cells.

In some embodiments, the composition used in the methods comprises less than 10% SOX9-positive cells. In some embodiments, the composition used in the methods comprises less than 10%, 9%, 8%, 7%, 6%, 5%, 4%, 3%, 2% or 1% SOX9-positive cells. In some embodiments, the composition used in the disclosed methods comprises 0.1-10%, 0.1-7%, 0.1-3%, 0.1-1%, 0.5-10%, 0.5-7%, 0.5-3%, 0.5-1%, 1-10%, 1-5%, 1-3%, 3-10%, 3-5%, or 5-10% SOX9-positive cells.

In some embodiments, the composition used in the methods comprises less than 5% Ki67-positive cells. In some embodiments, the composition used in the disclosed methods comprises less than 5%, 4%, 3%, 2% or 1% Ki67-positive cells. In some embodiments, the composition used in the disclosed methods comprises 0.01-0.1%, 0.1-5%, 0.1-3%, 0.1-1%, 0.5-5%, 0.5-3%, 0.5-1%, 1-5%, 1-3%, or 1-2% Ki67-positive cells.

In some embodiments, at least 80%, 85%, 90%, 91%, 92%, 93%, 94%, 95%, 96%, 97%, 98%, or 99% of the cells in the composition are CHGA-positive cells. In some embodiments, 80-100%, 85-100%, 90-100%, 90-99%, 90-98%, 95-99%, or 95-99% of the cells in the composition are CHGA-positive cells.

In some embodiments, the percentage of cells expressing a marker is measured by flow cytometry. The skilled worker is aware of representative methods for testing whether a cell or collection of cells is positive or negative for expression of a specific gene marker (e.g., NKX6.1, ISL1, INS, GCG, somatostatin, chromogranin A, SOX9, C-peptide, Ki67) by flow cytometry. In some embodiments, a cell is considered positive for expression of a particular gene (e.g., NKX6.1, ISL1, INS, GCG, somatostatin, chromogranin A, SOX9, C-peptide, Ki67) based on median fluorescence intensity (rMFI). As used herein, the term “rMFI” or relative median fluorescence intensity is the ratio between the fluorescence intensity measured by use of an antibody to a specific target (e.g., NKX6.1, ISL1, INS, GCG, somatostatin, chromogranin A, SOX9, C-peptide, Ki67) versus the intensity obtained from a control antibody (isotype control). In some embodiments, an anti-(human) NKX6.1, ISL1, INS, GCG, somatostatin, chromogranin A, or SOX9 antibody is used. Examples of suitable antibodies for use in flow cytometry are any of the antibodies disclosed in Table 1. An example of a suitable flow cytometer is the Accuri 6 flow cytometer. In some embodiments, the target-expressing cells (e.g., cells expressing NKX6.1 and/or ISL1), if tested, exhibit a target relative medium fluorescence intensity (rMFI) of at least 6, 6.5, 7, 8, 9 or 10 as measured by flow cytometry. In another embodiment, said rMFI is between 6.5 and 15, between 6.5 and 14, between 6.5 and 13, between 6.5 and 13, between 6.5 and 12, or between 6.5 and 10.

TABLE 1

| Primary | | | Primary | Secondary | |
|---|---|---|---|---|---|
| Antibody | Company | Cat# | Antibody Species | Antibody Species | Cat# |
| NKX6.1 | DSHB | FSSA12 | Mouse | anti-Mouse 488 | A21202 |
| IsL1 | abcam | ab178400 | Rabbit | anti-Rabbit-647 | A31573 |
| SST | Santa Cruz | SC-55565 | Anti-Somatostatin | ALEXAFLUOR ® 647 | — |
| | Biotechnology | AF647 | Antibody (G-10) | | |
| | | | ALEXAFLUOR ® 647 | | |
| Glu | R&D | IC 1249G | Human/Mouse | ALEXAFLUOR ®488 | — |
| | | | Glucagon | | |
| | | | ALEXAFLUOR ® | | |
| | | | 488-conjugated | | |
| Sox9 | Epitomics | AC-0284RUOC | Rabbit | anti-Rabbit-647 | A31573 |
| Ki67 | Thermo | | Ki-67 Monoclonal | PE | — |
| | Fisher | | Antibody (SolA15), | | |
| | | | PE | | |
| C-pep | DSHB | GN-1D4-S | Rat | anti-Rat-488 | A21208 |
| CHGA | abcam | ab15160 | Rabbit | anti-Rabbit-647 | A31573 |

In some embodiments, the percentage of cells expressing a marker can be measured by qRT-PCR or single cell RNA sequencing analysis. The skilled worker is aware of methods for testing whether a cell or collection of cells is positive for expression of a specific gene marker (e.g., NKX6.1, ISL1, INS, GCG, ARX, or ghrelin) by single cell RNA sequencing analysis.

In some embodiments, a population of in vitro differentiated cells used for the methods described herein comprises less than 25% (e.g., less than 25%, less than 20%, less than 15%, less than 10%, less than 5%, or less) of NKX6.1-positive, ISL1-negative cells. In some embodiments, a population of in vitro differentiated cells described herein comprises about 2%-25%, 2%-20%, 2%-15%, 2%-10%, 2%-5%, 5%-25%, 5%-20%, 5%-15%, 5%-10%, 10%-25%, 10%-20%, 10%-15%, 15%-25%, 15%-20%, or 20%-25% of NKX6.1-positive, ISL1-negative cells. In some embodiments, a population of in vitro differentiated cells described herein comprises about 1-2%, 2%-10%, 2%-8%, 2%-6%, 2%-4%, 4%-10%, 4%-8%, 4%-6%, 6%-10%, 6%-8%, or 8%-10% of NKX6.1-positive, ISL1-negative cells. In some embodiments, a population of in vitro differentiated cells described herein comprises about 2%, 4%, 6%, 8%, or 10% of NKX6.1-positive, ISL1-negative cells.

In some embodiments, the disclosure provides methods for using a pharmaceutical composition comprising NKX6.1-positive, ISL1-positive cells that express lower levels of MAFA than NKX6.1-positive, ISL1-positive cells from the pancreas of a healthy control adult subject or from a cadaveric pancreas. In some embodiments, the pharmaceutical composition comprises NKX6.1-positive, ISL1-positive cells that express higher levels of MAFB than NKX6.1-positive, ISL1-positive cells from the pancreas of a healthy control adult subject or from a cadaveric pancreas. In some embodiments, the pharmaceutical composition comprises NKX6.1-positive, ISL1-positive cells that express higher levels of SIX2, HOPX, IAPP and/or UCN3 than NKX6.1-positive, ISL1-positive cells from the pancreas of a healthy control adult subject or from a cadaveric pancreas. In some embodiments, the pharmaceutical composition comprises NKX6.1-positive, ISL1-positive cells that do not express MAFA. In some embodiments, the pharmaceutical composition comprises NKX6.1-positive, ISL1-positive cells that express MAFB. In some embodiments, the pharmaceutical composition comprises cells that are genetically modified (e.g., using a gene editing technology such as CRISPR). In some embodiments, the pharmaceutical composition comprises NKX6.1-positive, ISL1-positive cells that express lower levels of beta-2 microglobulin, CIITA, HLA-A, HLA-B, HLA-C, HLA-DP, HLA-DQ, and HLADR than NKX6.1-positive, ISL1-positive cells from the pancreas of a healthy control adult subject or from a cadaveric pancreas. In some embodiments, the pharmaceutical composition comprises NKX6.1-positive, ISL1-positive cells that express increased levels of CD47, PDL1, HLA-G, CD46, CD55, CD59 and CTLA than NKX6.1-positive, ISL1-positive cells from the pancreas of a healthy control adult subject or from a cadaveric pancreas. In some embodiments, any of the cell markers disclosed herein (e.g., NKX6.1, PDX1, MAFA, MAFB, SIX2, HOPX, IAPP and/or UCN3) are detected by flow cytometry.

In some embodiments, the disclosure provides for a method of treating a subject having diabetes, comprising administering any of the cell compositions disclosed herein (e.g., a composition comprising a population of stem cell-derived islet cells) to a subject that had previously received an organ transplant. In some embodiments, the method also comprises administering to the subject one or more of any of the immunosuppressive agents disclosed herein. In some embodiments, the cell composition comprises about 1×10⁸ to about 8×10⁸, about 1×10⁸ to about 7×10⁸, about 1×10⁸ to about 6×10⁸, about 1×10⁸ to about 5×10⁸, about 1×10⁸ to about 4×10⁸, about 1×10⁸ to about 3×10⁸, about 1×10⁸ to about 2×10⁸, about 1.5×10⁸ to about 6.5×10⁸, about 2×10⁸ to about 7×10⁸, about 2×10⁸ to about 6×10⁸, about 2×10⁸ to about 5×10⁸, about 2×10⁸ to about 4×10⁸, about 2×10⁸ to about 3×10⁸, about 2.5×10⁸ to about 5.5×10⁸, about 3×10⁸ to about 7×10⁸, about 3×10⁸ to about 6×10⁸, about 3×10⁸ to about 5×10⁸, about 3×10⁸ to about 4×10⁸, about 3.5×10⁸ to about 4.5×10⁸, about 3.8×10⁸ to about 4.2×10⁸ cells, about 7.5×10⁸ to about 10×10⁸ cells, or about 8×10⁸ to about 12×10⁸ cells. In particular embodiments, the cell composition comprises between 7×10⁸ and 9×10⁸ cells. In some embodiments, the subject is administered a first dose comprising any of the cell compositions disclosed herein (e.g., a cell composition comprising 7×10⁸ and 9×10⁸ SC-islet cells) and is administered one or more subsequent doses of any of the cell compositions disclosed herein (e.g., a cell composition comprising about 3.8×10⁸ to about 4.2×10⁸ SC-islet cells or about 7×10⁸ to about 9×10⁸ SC-islet cells). In some embodiments, the first and subsequent doses are the same or about the same (e.g., about 8×10⁸ SC-islet cells). In some embodiments, subjects may receive a second dose if the subject continues to require exogenous insulin to achieve glycemic targets. In some embodiments, a subject may receive a subsequent dose if the subject restarts use of exogenous insulin after being free from exogenous insulin. In some embodiments, subjects will not receive a subsequent dose if the subject restarts use of exogenous insulin due to a temporary, reversible condition (e.g., intercurrent illness for a period of ≤14 days).

In some embodiments, the disclosure provides for a composition comprising any of the cell populations described in any of WO2012175633; WO2015028614; WO2017144695; WO2019048690; WO2020207998; WO2022043518; WO2023203208; WO2024008810; WO2025029826; WO2025093467; Wang et al., 2024, Cell, 187:1-13; Wu et al., 2024, Cell Discovery, 10 (45); WO2025117331; U.S. Pat. No. 11,274,280; Hogrebe et al., Nat Protoc., 2021, 16 (9): 4109-4143; Vegas et al., 2016, Nat Med. 2016 March; 22 (3): 306-311; Wu et al., 2024, Cell Discovery, 10 (45); and Ratiu et al., 2023, bioRxiv, doi.org/10.1101/2023.10.20.563345, each of which is herein incorporated by reference in its entirety.

In some embodiments, the pharmaceutical composition is derived from stem cells in vitro. In some embodiments, the stem cells are genetically modified (e.g., using a gene editing technology such as CRISPR). In some embodiments, the stem cells have reduced expression of one or more of beta-2 microglobulin, CIITA, HLA-A, HLA-B, HLA-C, HLA-DP, HLA-DQ, and HLADR, relative to stem cells that are not genetically modified. In some embodiments, the stem cells have increased expression of CD47, PDL1, HLA-G, CD46, CD55, CD59 and CTLA, relative to stem cells that are not genetically modified. In some embodiments, any of the cell markers disclosed herein (e.g., NKX6.1, PDX1, MAFA, MAFB, SIX2, HOPX, IAPP and/or UCN3) are detected by flow cytometry.

In some cases, cell populations or cell clusters are unsorted, e.g., isolated cell populations or cell clusters that have not been through cell sorting process. In some embodiments, the cell clusters can refer to a cell cluster formed by self-aggregation of cells cultured in a given environment, for instance, in a 3D suspension culture. Cell sorting as described herein can refer to a process of isolating a group of cells from a plurality of cells by relying on differences in cell size, shape (morphology), surface protein expression, endogenous signal protein expression, or any combination thereof. In some cases, cell sorting comprises subjecting the cells to flow cytometry. Flow cytometry can be a laser- or impedance-based, biophysical technology. During flow cytometry, one can suspend cells in a stream of fluid and pass them through an electronic detection apparatus. In one type of flow cytometry, fluorescent-activated cell sorting (FACS), based on one or more parameters of the cells' optical properties (e.g., emission wave-length upon laser excitation), one can physically separate and thereby purify cells of interest using flow cytometry. As described herein, an unsorted cell cluster can be cell cluster that formed by a plurality of cells that have not been subject to an active cell sorting process, e.g., flow cytometry. In some cases, flow cytometry as discussed herein can be based on one or more signal peptides expressed in the cells. For example, a cell cluster can comprise cells that express a signal peptide (e.g., a fluorescent protein, e.g., green fluorescent protein (GFP) or tdTomato). In some cases, the signal peptide is expressed as an indicator of insulin expression in the cells. For instance, a cell cluster can comprise cell harboring an exogenous nucleic acid sequence coding for GFP under the control of an insulin promoter. The insulin promoter can be an endogenous or exogenous promoter. In some cases, the expression of GFP in these cells can be indicative of insulin expression in the cells. The GFP signal can thus be a marker of a pancreatic β cell. In some cases, cell sorting as described herein can comprise subjecting cells to magnetic-activated sorting process, where magnetic antibody or other ligand is used to label cells of different types, and the differences in magnetic properties can be used for cell sorting.

The percentage of cells expressing one or more particular markers, like PDX1, NKX6.1, insulin, NGN3, or CHGA, described herein can be the percentage value detected using techniques like flow cytometry assay. In some cases, during a flow cytometry assay, cell population or cell cluster discussed herein are dispersed into single-cell suspension by incubation in digesting enzyme like trypsin or TRYPLE™ Express. Dispersed cell can be washed in suitable buffer like PBS, centrifuged and then re-suspended in fixation buffer like 4% PFA. Incubation with primary antibodies against the cell markers of interest can then be conducted, which can be followed by incubation with the secondary antibodies. After antibody incubation, the cells can be washed and the subject to segregation by flow cytometry. Techniques other than flow cytometry can also be used to characterize the cells described herein, e.g., determine the cell percentages. Non-limiting examples of cell characterization methods include gene sequencing, microscopic techniques (fluorescence microscopy, atomic force microscopy), karyotyping, isoenzyme analysis, DNA properties, and viral susceptibility.

In some embodiments, any of the cells used in the methods disclosed herein comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, any of the cells used with the methods disclosed herein (e.g., any of the SC-β cells or cells in any of the clusters disclosed herein) comprise a genomic disruption in at least one gene sequence, wherein said disruption reduces or eliminates expression of a protein encoded by said gene sequence. In some embodiments, said at least one gene sequence encodes an MHC-Class I gene. In some embodiments, said MHC-Class I gene encodes beta-2 microglobulin (B2M), HLA-A, HLA-B, or HLA-C. In some embodiments, said at least one gene sequence encodes CIITA. In some embodiments, the cells comprise a genomic disruption in the genes encoding HLA-A and HLAB, but do not comprise a genomic disruption in the gene encoding HLA-C. In some embodiments, said cells comprise a genomic disruption in a natural killer cell activating ligand gene. In some embodiments, said natural killer cell activating ligand gene encodes intercellular adhesion molecule 1 (ICAM1), CD58, CD155, carcinoembryonic antigen-related cell adhesion molecule 1 (CEACAM1), cell adhesion molecule 1 (CADM1), MHC-Class I polypeptide-related sequence A (MICA), or MHC-Class I polypeptide-related sequence B (MICB). In some embodiments, the cells have reduced expression of one or more of beta-2 microglobulin, CIITA, HLA-A, HLA-B, HLA-C, HLA-DP, HLA-DQ, and HLADR, relative to cells that are not genetically modified. In some embodiments, the cells have increased expression of CD47, PDL1, HLA-G, CD46, CD55, CD59 and CTLA, relative to cells that are not genetically modified. In particular embodiments, the pancreatic islet cells herein (e.g., the SC-beta cells) have increased expression of PDL1 as compared to endogenous pancreatic islet cells from a healthy control subject. In particular embodiments, the pancreatic islet cells (e.g., the SC-beta cells) have increased expression of CD47 as compared to endogenous pancreatic islet cells from a healthy control subject. In some embodiments, the genomic disruption is induced by use of a gene editing system, e.g., CRISPR Cas technology.

In some embodiments, any of the cells disclosed herein comprise a genomic disruption in at least one gene sequence, wherein said disruption reduces or eliminates expression of a protein encoded by said gene sequence (e.g., by knocking out one or both functional copies of a gene in a subject). In some embodiments, said at least one gene sequence is the ABO sequence, such that the disruption results in the cell being blood type O. In some embodiments, said at least one gene sequence encodes an MHC-Class I gene. In some embodiments, said MHC-Class I gene encodes beta-2 microglobulin (B2M), HLA-A, HLA-B, or HLA-C. In some embodiments, said at least one gene sequence encodes CIITA. In some embodiments, the cells comprise a genomic disruption in the genes encoding HLA-A and HLA-B, but do not comprise a genomic disruption in the gene encoding HLA-C. In some embodiments, the cells comprise a genomic disruption in the gene encoding CXCL10. In some embodiments, the cells comprise a genomic disruption in the gene encoding renalase. In some embodiments, a cell comprises disrupted expression of B2M, CXCL10, renalase, tissue factor, and/or ABO, and/or comprises increased expression or activity of CD47. In some embodiments, said cells comprise a genomic disruption in a natural killer cell activating ligand gene. In some embodiments, said natural killer cell activating ligand gene encodes intercellular adhesion molecule 1 (ICAM1), CD58, CD155, carcinoembryonic antigen-related cell adhesion molecule 1 (CEACAM1), cell adhesion molecule 1 (CADM1), MHC-Class I polypeptide-related sequence A (MICA), or MHC-Class I polypeptide-related sequence B (MICB). In some embodiments, the cells have reduced expression of one or more of beta-2 microglobulin, CIITA, HLA-A, HLA-B, HLA-C, HLA-DP, HLA-DQ, and HLADR, relative to stem cells that are not genetically modified. In some embodiments, the cells have reduced expression of one or more of beta-2 microglobulin, CIITA, HLA-A, HLA-B, HLA-C, HLA-DP, HLA-DQ, and HLADR, relative to cells that are not genetically modified. In some embodiments, the cells have increased expression of CD47, PDL1, HLA-G, CD46, CD55, CD59, CTLA, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9 relative to cells that are not genetically modified. In particular embodiments, the pancreatic islet cells disclosed herein (e.g., the SC-beta cells) have increased expression of PDL1 as compared to endogenous pancreatic islet cells from a healthy control subject. In particular embodiments, the pancreatic islet cells herein (e.g., the SC-beta cells) have increased expression of CD47 as compared to endogenous pancreatic islet cells from a healthy control subject. In some embodiments, the genomic disruption is induced by use of a gene editing system, e.g., CRISPR Cas technology. In some embodiments, any of the isolated cells (e.g., a stem cell or a NKX6.1-positive, ISL1-positive cell) described herein comprises a disruption (e.g., deletion, insertion, translocation, inversion, or substitution of one or more nucleotides) in any one or more of the genes encoding: B2M, CIITA, CXCL10, renalase, HLA-A, HLA-B, HLA-C, RFX-ANK, NFY-A, NLRC5, RFX5, RFX-AP, HLA-G, HLA-E, NFY-B, PD-L1, NFY-C, IRF1, TAPI, GITR, 4-1BB, CD28, B7-1, CD47, B7-2, 0X40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, IDO1, TRAC, TRB, NFY-A, CCR5, F3, CD142, MICA, MICB, LRP1, HMGB1, ABO, RHD, FUT1, KDM5D, PDGFRa, OLIG2, and/or GFAP. In some embodiments, disruption of a gene results in an at least 10%, 20%, 30%, 40%, 50%, 60%, 70%, 80%, 90% or 100% decrease in expression of the gene as compared to the expression of the gene in the same type of cell without the disruption. In some embodiments, the gene is disrupted using CRISPR/Cas, piggybac transposon, TALEN, and/or zinc finger technology. In some embodiments, the cell is any of the gene edited cells disclosed in WO2023070019 or WO2024097697, each of which is incorporated by reference herein in its entirety.

In some embodiments, a cell (e.g., an isolated stem cell or a NKX6.1-positive, ISL1-positive cell) is negative for A antigen and negative for B antigen. In some embodiments, the cell is negative for A antigen. In some embodiments, the cell is negative for B antigen. In some embodiments, a cell (e.g., an isolated stem cell or a NKX6.1-positive, ISL1-positive cell) is negative for Rh antigen. In some embodiments, a cell (e.g., an isolated stem cell or a NKX6.1-positive, ISL1-positive cell) is negative for A antigen, negative for B antigen, and negative for Rh antigen. An “A antigen,” as used herein, refers to a histo-blood group antigen produced by 3α-N-acetylgalactosaminyltransferase and expressed as a cell-surface antigen. A “B antigen,” as used herein, refers to a histo-blood group antigen produced by 3α-galactosaminyltransferase and expressed as a cell-surface antigen. In some embodiments, the cell comprises a disruption in the ABO gene. In some embodiments, the cell comprises a disruption in the ABO gene such that the cell has reduced or absent levels of A and B antigens. In some embodiments, the cell comprises a disruption in the FUT1 gene. In some embodiments, the cell comprises a disruption in the FUT1 gene such that Galactoside 2-alpha-L-fucosyltransferase 1 expression is reduced or absent. An “Rh antigen,” as used herein, refers to a highly immunogenic antigen encoded by two highly polymorphic genes, RHD and RHCE. Rh antigen proteins are transmembrane proteins. In some embodiments, the cell comprises a disruption in the RHAG gene. In some embodiments, the cell comprises a disruption in the RHAG gene such that the cell has reduced or absent levels of Rh-associated glycoprotein. In some embodiments, the cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

In some embodiments, any of the cells herein (e.g., any of the SC-islet cells disclosed herein) comprises a “safety switch.” In some embodiments, the safety switches are nucleic acid constructs encoding a switch protein that inducibly causes cell death or stops cell proliferation. In some embodiments, the safety switch is inserted at a defined, specific target locus (e.g., a safe harbor locus) in the genome of an engineered cell, usually at both alleles of the target locus. In some embodiments, the target locus is a safe harbor locus, such as ActB or CLYBL. In some embodiments, the target locus is a gene targeted for disruption (e.g., B2M or CIITA). In some embodiments, the switch protein is activated by contacting with an effective dose of a clinically acceptable orthologous small molecule. In some embodiments, when activated, the safety switch causes the cell to stop proliferation, in some embodiments by activating apoptosis of the cell. In some embodiments, the switch protein comprises herpes-simplex-thymidine-kinase. In some embodiments the switch protein comprises a human caspase protein, e.g., caspase 1, caspase 2, caspase 3, caspase 4, caspase 5, caspase 6, caspase 7, caspase 8, caspase 9, caspase 10, caspase 14, etc. In certain embodiments the protein is human caspase 9. In some embodiments, the caspase protein is fused to a sequence that provides for chemically induced dimerization (CID), in which dimerization occurs only in the presence of the orthologous activating agent. One or more CID domains may be fused to the caspase protein, e.g., two different CID domains may be fused to the caspase protein. In some embodiments the CID domain is a dimerization domain of FKBP or FRB (FKBP-rapamycin-binding) domain of mTOR, which are activated with rapamycin analogs. In some embodiments, the safety switch is any of the safety switches described in WO2021173449 and Jones et al., 2014, Frontiers in Pharmacology, 5(254): 1-8, each of which is incorporated herein in its entirety.

In some embodiments of the methods disclosed herein, at least a portion of the cells in the population of cells are present in plurality of cell clusters. In some cases, the cell clusters are about 50 μm to about 500 μm, about 50 μm to about 400 μm, about 50 μm to about 300 μm, about 60 μm to about 400 μm, about 60 μm to about 300 μm, about 60 μm to about 250 μm, about 75 μm to about 400 μm, about 75 μm to about 300 μm, about 75 μm to about 250 μm, about 125 μm to about 225 μm, about 130 μm to about 160 μm, about 170 μm to about 225 μm, about 140 μm to about 200 μm, about 140 μm to about 170 μm, about 160 μm to about 220 μm, about 170 μm to about 215 μm, or about 170 μm to about 200 μm in diameter. In some cases, the population of cells are present as a single cell suspension. In some embodiments, at least 50%, at least 60%, at least 70%, at least 75%, at least 80%, at least 85%, at least 90%, at least 92%, at least 94%, at least 95%, at least 96%, at least 98%, or at least 99% of the cells are present in cell clusters. In some embodiments, substantially all of the cells are present in cell clusters, e.g., at least 99.5%, at least 99.6%, at least 99.7%, at least 99.8%, at least 99.9%, at least 99.9%, at least 99.99%, at least 99.999%, or at least 99.9999% of the cells. In some embodiments, the cells are dissociated. In some embodiments, at least 50%, at least 60%, at least 70%, at least 75%, at least 80%, at least 85%, at least 90%, at least 92%, at least 94%, at least 95%, at least 96%, at least 98%, or at least 99% of the cells are dissociated. In some embodiments, substantially all of the cells are dissociated, e.g., at least 99.5%, at least 99.6%, at least 99.7%, at least 99.8%, at least 99.9%, at least 99.9%, at least 99.99%, at least 99.999%, or at least 99.9999% of the cells.

In some embodiments, disclosure provides for a method using a composition comprising a population of insulin-positive cells and a lipid. In some embodiments, the disclosure provides for a method of contacting a population of insulin-positive cells with a lipid. In some embodiments, the lipid is a saturated fatty acid. In some embodiments, the saturated fatty acid is palmitate. In some embodiments, the lipid is an unsaturated fatty acid. In some embodiments, the non-saturated fatty acid is oleic acid, linoleic acid, or palmitoleic acid.

In some embodiments, the disclosure provides for a method using a composition comprising a population of insulin-positive cells and MCDB 131. In some embodiments, the disclosure provides for a method of contacting a population of insulin-positive cells with MCDB 131. In some embodiments, the disclosure provides for a method using a composition comprising a population of insulin-positive cells and DMEM/F12. In some embodiments, the disclosure provides for a method of contacting a population of insulin-positive cells with DMEM/F12. In some embodiments, the disclosure provides for a method using a composition comprising a population of insulin-positive cells and zinc. In some embodiments, the disclosure provides for a method of contacting a population of insulin-positive cells with zinc. In some embodiments, the disclosure provides for a method using a composition comprising a population of insulin-positive cells and ZnSO₄. In some embodiments, the disclosure provides for a method of contacting a population of insulin-positive cells with ZnSO₄.

In some embodiments, the disclosure provides for a method using a composition comprising a population of insulin-positive cells and at least one metabolite. In some embodiments, the disclosure provides for a method of contacting a population of insulin-positive cells with at least one metabolite. In some embodiments, the at least one metabolite is glutamate, acetate, b-hydroxybutyrate, L-carnitine, taurine, formate, or biotin. In some embodiments, the disclosure provides for a method using a composition comprising a population of insulin-positive cells and one, two, three, four, five, six, or seven of glutamate, acetate, b-hydroxybutyrate, L-carnitine, taurine, formate, or biotin. In some embodiments, the disclosure provides for a method of contacting a population of insulin-positive cells with one, two, three, four, five, six, or seven of glutamate, acetate, b-hydroxybutyrate, L-carnitine, taurine, formate, or biotin.

In some embodiments, the disclosure provides for a method using a composition comprising a population of insulin-positive cells and at least one amino acid. In some embodiments, the disclosure provides for a method of contacting a population of insulin-positive cells with at least one amino acid. In some embodiments, the at least one amino acid is alanine, glutamate, glycine, proline, threonine, or tryptophan. In some embodiments, the at least one amino acid is arginine, histidine, lysine, aspartic acid, glutamic acid, serine, asparagine, glutamine, cysteine, selenocysteine, alanine, valine, isoleucine, leucine, methionine, phenylalanine, tyrosine, glutamate, glycine, proline, threonine, or tryptophan. In some embodiments, the disclosure provides for a method using a composition comprising a population of insulin-positive cells and at least one vitamin. In some embodiments, the disclosure provides for a method of contacting a population of insulin-positive cells with at least one vitamin. In some embodiments, the at least one vitamin is biotin or riboflavin.

In some embodiments, the disclosure provides for a method using a composition comprising a population of insulin-positive cells and a monoglyceride lipase (MGLL) inhibitor. In some embodiments, the disclosure provides for a method of contacting a population of insulin-positive cells with at least one vitamin. In some embodiments, the MGLL inhibitor is any of JJKK048, KML29, NF1819, JW642, JZL184, JZL195, JZP361, pristimerin, or URB602, or derivatives thereof.

### Systems and Kits

Also provided are systems and kits that can be used with the disclosed methods. In some examples, the system or kit includes a population of stem cell-derived islet cells and/or one or more immunosuppressive agents, for example with a pharmaceutically acceptable carrier. In some examples, the system or kit further includes a premedication, an immune response modulator, a blood thinner, or an antimicrobial agent. In some examples, the system or kit includes an analgesic (e.g., acetaminophen), an antipyretic (e.g., acetaminophen), an antihistamine (e.g., diphenhydramine), a corticosteroid (e.g., methylprednisolone), or a hemorrheologic agent (e.g., pentoxyfilline). In some examples, the system or kit includes an anti-thymocyte globulin binding moiety. In some examples, the system or kit includes a TNFα inhibitor, such as etanercept.

In some examples, the population of stem cell-derived islet cells and one or more immunosuppressive agents, premedication, immune response modulator, blood thinner, antimicrobial agent, analgesic, antipyretic, antihistamine, corticosteroid hemorrheologic agent, anti-thymocyte globulin binding moiety, or TNFα inhibitor are provided in a single container. In some examples, the population of stem cell-derived islet cells and one or more immunosuppressive agents, premedication, immune response modulator, blood thinner, antimicrobial agent, analgesic, antipyretic, antihistamine, corticosteroid hemorrheologic agent, anti-thymocyte globulin binding moiety, or TNFα inhibitor are provided in separate containers, suitable to be combined prior to administration to a subject. In some examples, the population of stem cell-derived islet cells and one or more immunosuppressive agents, premedication, immune response modulator, blood thinner, antimicrobial agent, analgesic, antipyretic, antihistamine, corticosteroid hemorrheologic agent, anti-thymocyte globulin binding moiety, or TNFα inhibitor are provided in separate containers suitable to be administered concurrently, or subsequently to a subject. The container(s) can be any suitable container, such as a syringe, IV bag, ampule, dropper, a vessel, a vial, a bottle, a canister, a blister pack, a carton, a spray bottle, a tube, a metered dose container, an autoinjector, or a combination of the forgoing.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1. Infusion of Non-Native Pancreatic Cells to Treat Diabetes in Human Organ Transplant Recipients

This example illustrates methods of treating of human subjects with Type I diabetes, who previously received an organ transplant, by infusing non-native pancreatic cells (also termed as “stem cell-derived islet cells” or “SC-islet cells” hereafter) according to some embodiments of the present disclosure. In the following example, the subjects are kidney transplant recipients, however it is understood that the following methods illustrate methods of treating human subjects with Type I diabetes who previously received other types of organ transplants (e.g., liver transplant recipients). In this example, prior to being infused with the SC-islet cells, the patient will have received one or more immunosuppressant (e.g., tacrolimus and/or mycophenolate mofetil).

SC-islet cells, including mature, non-native β cells capable of releasing insulin in response to glucose challenge in vitro, are generated by an in vitro differentiation process. The differentiation process follows a 6-stage stepwise protocol, starting from human embryonic stem cells to generation of mature SC-islet cells, as outlined below in Table 2.

TABLE 2
Differentiation Media Stages 1-6 (S1-S6)-

| Stage#Day# | Reagents Included |
|---|---|
| S0D1-2 | bFGF (100 ng/ml) |
| S1D1 | Activin-A (100 ng/ml) |
| | CHIR99021 (3 μM) |
| | Ascorbic Acid (0.088 mg/ml) |
| | ITS-X (20 μg/L) |
| | PVA80 |
| S1D2-3 | Activin-A (100 ng/ml) |
| | Ascorbic Acid (0.088 mg/ml) |
| | ITS-X (20 μg/L) |
| | PVA80 |
| S2D1-3 | KGF (50 ng/ml) |
| | PVA80 |
| | Ascorbic Acid (0.088 mg/ml) |
| | ITS-X (20 μg/L) |
| S3D1 | KGF (50 ng/ml) |
| | PDBU (500 nM) |
| | Sant-1 (250 nM) |
| | Retinoic Acid (2 μM) |
| | Activin A (20 ng/ml) |
| | Thiazovinin (2.5 μM) |
| | DMH-1 (250 nM) |
| | PVA80 |
| | Ascorbic Acid (0.088 mg/ml) |
| | ITS-X (5 mg/L) |
| S3D2 | KGF (50 ng/ml) |
| | PDBU (500 nM) |
| | Sant-1 (250 nM) |
| | Retinoic Acid (2 μM) |
| | Activin A (20 ng/ml) |
| | Thiazovinin (2.5 μM) |
| | PVA80 |
| | Ascorbic Acid (0.088 mg/ml) |
| | ITS-X (5 mg/L) |
| S4D1-4 | KGF (50 ng/ml) |
| | Sant-1 (250 nM) |
| | Thiazovinin (2.5 μM) |
| | Activin A (5 ng/ml) |
| | Retinoic Acid (100 nM) |
| | PVA80 |
| | Ascorbic Acid (0.088 mg/ml) |
| | ITS-X (5 mg/L) |
| S4D5-6 | KGF (50 ng/ml) |
| | Sant-1 (250 nM) |
| | Thiazovinin (2.5 μM) |
| | Activin A (5 ng/ml) |
| | Retinoic Acid (100 nM) |
| | PDBU (500 nM) |
| | AS1842856 (1 μM) |
| | XXI (2 μM) |
| | PVA80 |
| | Ascorbic Acid (0.088 mg/ml) |
| | ITS-X (5 mg/L) |
| S5D1-3 | Sant-1 (250 nM) |
| | Betacellulin (20 ng/ml) |
| | XXI (2 μM) |
| | Alk5i (10 μM) |
| | GC-1 (1 μM) |
| | LDN-193189 (100 nM) |
| | Thiazovinin (2.5 μM) |
| | Staurosporine (3 nM) |
| | DZNEP (100 nM) |
| | Retinoic Acid (50 nM) |
| | PDBU (500 nM) |
| | NVP (2 μM) |
| | L-glutamine (4 mM) |
| | Formate (50 μM) |
| | Taurine (90 μM) |
| | Acetate (1 mM) |
| | β-hydroxybutyrate (200 nM) |
| | Biotin (800 nM) |
| | PVA 87-89 |
| | Ascorbic Acid (0.088 mg/ml) |
| | ITS-X (5 mg/L) |
| | ZnSO4 (0.002857 mg/ml) |
| S5D4-7 | XXI (2 μM) |
| | Alk5i (10 μM) |
| | GC-1 (1 μM) |
| | LDN-193189 (100 nM) |
| | Thiazovinin (2.5 μM) |
| | Staurosporine (3 nM) |
| | DZNEP (100 nM) |
| | L-glutamine (4 mM) |
| | Formate (50 μM) |
| | Taurine (90 μM) |
| | Acetate (1 mM) |
| | β-hydroxybutyrate (200 nM) |
| | Biotin (800 nM) |
| | PVA 87-89 |
| | Ascorbic Acid (0.088 mg/ml) |
| | ITS-X (5 mg/L) |
| | ZnSO4 (0.002857 mg/ml) |
| S6D1-3 | HSA 0.05% (v/v) |
| | ALK5 Inhibitor II (10 μM) |
| | DZNeP (100 nM) |
| | GC-1 (1 μM) |
| | LDN-193189 (100 nM) |
| | Staurosporine (3 nM) |
| | Thiazovivin (2.5 μM) |
| | L-Glutamic Acid (500 μM) |
| | L-Carnitine (40 μM) |
| | Taurine (90 μM) |
| | Sodium Acetate (160 nM) |
| | β-hydroxybutyrate (200 nM) |
| | Biotin (800 nM) |
| | Sodium Formate (50 μM) |
| | Zinc Sulfate (10 μM) |
| S6D4-5 | HSA 1% (v/v) |

Cells are dissociated and cryopreserved following Stage 5 (S5) and then thawed and reaggregated for Stage 6. Stage 6 is performed to generate SC-islets. Type 1 diabetic subjects who previously received a kidney transplant and who are already receiving one or more immunosuppressant agents (e.g., tacrolimus or mycophenolate mofetil) are selected for SC-islet treatment. Subjects are also selected who are taking a stable immunosuppression regimen of tacrolimus and mycophenolate mofetil, mycophenolate sodium, or sirolimus for at least 4 weeks. The regimen may include prednisone ≤5 mg per day. Subjects are premedicated before infusion of SC-islets, unless a medical contraindication exists. Subjects may be premedicated with both H1 and H2 antihistaminergic medications before infusion of SC-islets (e.g., diphenhydramine [H1 antihistamine; 50 mg] and famotidine [H2 antihistamine; 20 mg], up to 1 hour before infusion of SC-islets). These medications may be repeated as needed. SC-islets are delivered slowly via gravity drainage through a catheter in the portal vein or over a targeted time for infusion of approximately 60 minutes for 0.8×10⁹ total SC-islet cells.

Subjects can be treated according to an immunosuppressive/additional treatment regimen as described in the protocol below.

### Peri-Infusion of SC-Islets:

Subjects will continue the immunosuppression regimen used for their kidney transplant. If immunosuppression needs to be changed because it is not tolerated, subjects may be transitioned to a combination of tacrolimus, mycophenolate mofetil, mycophenolate sodium, sirolimus, and prednisone ≤5 mg daily, with the goal of protecting the renal allograft and the SC-islets. An alternative regimen may be used to protect the renal allograft if necessary. This regimen is temporary, if possible, and may include immunosuppressants such as azathioprine, belatacept, cyclosporine, or everolimus.

Tacrolimus will be titrated beginning on Day −2 and no later than Day 1, targeting a whole blood trough of 8 to 10 ng/ml through Day 90 and 6 to 8 ng/mL after Day 90. If the subject is taking sirolimus, the target tacrolimus trough is 3 to 6 ng/mL.

If a subject is taking sirolimus, sirolimus will be titrated beginning on Day −2 and no later than Day 1, targeting a whole blood 24-hour trough of 10 to 15 ng/mL through Day 90 and then 8 to 12 ng/ml after Day 90.

If a subject is taking mycophenolate mofetil or mycophenolate sodium, the following doses may be continued: mycophenolate mofetil: 500 to 1000 mg oral (PO) twice per day (bid); mycophenolate sodium: 360 to 720 mg PO bid.

### Induction:

Rabbit anti-thymocyte globulin (THYMOGLOBULIN®; ATG) will be used for induction for subjects who were not previously treated with T-cell depleting therapy (TCDT; e.g., ATG or alemtuzumab). The total dose of ATG is 3 mg/kg given as an IV infusion starting on Day −2 through Day −1. The 1^{st} dose is 0.5 mg/kg infused over 6 to 12 hours on Day −2, followed by a rest period of ≥6 hours, followed by a 2^{nd} dose on Day −1 of 1.0 mg/kg infused over ≥6 hours, followed by a rest period of ≥6 hours, followed by a 3^{rd} dose on Day −1 of 1.5 mg/kg infused over ≥6 hours. The doses will be administered prior to the SC-islet infusion procedure, which will occur approximately 12 hours after the completion of the 3^{rd} dose. The following pre-medications are administered before/during infusion of ATG:

1. Acetaminophen/paracetamol: 500 mg to 650 mg PO/rectal (PR), approximately 30 minutes before and 500 mg to 650 mg mid-way through ATG infusion.

2. Diphenhydramine: 50 mg PO, approximately 30 minutes before and 50 mg mid-way through ATG infusion. In regions where diphenhydramine is not available, it may be substituted with another H1 antihistamine per institutional standards (e.g., clemastine, chlorphenamine).

3. Methylprednisolone: 1 mg/kg IV, approximately 1 hour before and as needed during first ATG infusion only on Day −2.

4. Pentoxifylline: 400 mg PO 3 times daily (tid) approximately 1 hour before first ATG infusion on Day −2 and continued through Day 8. However, if the subject previously received T-cell depleting therapy (TCDT; e.g., ATG or alemtuzumab), then subjects will receive Basiliximab for induction. Basiliximab will be administered 20 mg IV on Day 1 (≤2 hours before infusion) and on Day 5, diluted to 50 mL with normal saline and infused over approximately 20 to 30 minutes, per package insert.

If the subject is receiving more than one dose of SC-islet cells, basiliximab is used during the second SC-islet infusion and is administered 20 mg IV on Day 1 (≤2 hours before infusion) and on Day 5, diluted to 50 mL with normal saline and infused over approximately 20 to 30 minutes, per package insert.

Etanercept is administered peri-transplant 50 mg IV on Day 1 (approximately 1 hour before SC-islet infusion), then 25 mg subcutaneously on Days 4, 8, and 11. Alternatively, etanercept may be administered subcutaneously. Maintenance immunosuppression and monitoring is continued through the end of the study or until discontinuation of immunosuppression therapy.

Antimicrobial prophylaxis is administered as described below or may be superseded by local guidelines at the discretion of the clinician:

Trimethoprim/Sulfamethoxazole: Trimethoprim/sulfamethoxazole will be administered at a dose of 80 mg/400 mg PO once daily (qd) beginning on Day 2 for 6 months after SC-islet infusion. If a subject is unable to take trimethoprim/sulfamethoxazole, they will be treated on a case-by-case basis as medically indicated.

Clotrimazole: Clotrimazole will be administered as 1 troche PO 4 times per day (qid) beginning on Day −1 before the SC-islet infusion when basiliximab is used for induction (Day −2 when induction is with ATG) and continuing for 3 months after the last SC-islet dose. Alternatively, antifungal prophylaxis per standard practice at each site may be administered instead of clotrimazole.

Valganciclovir: Valganciclovir will be administered at a dose of 450 mg PO qd beginning on Day −1 before the SC-islet dose when basiliximab is used for induction (Day −2 when induction is with ATG) increasing to 900 mg qd by Day 13 and continuing for at least 6 months after SC-islet infusion. If the subject is cytomegalovirus (CMV) negative and the transplanted kidney was from a CMV negative donor, viral prophylaxis may be substituted by acyclovir at a dose of 400 mg PO bid.

Heparin: Heparin will be administered at a dose of 70 U/kg body weight of recipient, divided equally among the islet bags, given with SC-islet infusion, followed by 3 U/kg/h IV for the next 4 hours. From the 5th through the 48th hour post-transplant, heparin will be titrated to achieve and maintain activated partial thromboplastin time (aPTT) between 50 to 60 seconds. At the investigator's discretion, a comparable site-specific method and value may be used instead.

Enoxaparin Enoxaparin will be administered at a dose of 30 mg subcutaneously bid, with the first dose given 48 hours after the transplant procedure (when heparin is discontinued). Enoxaparin will be continued through Day 7 after SC-islet infusion. The dose can be modified or extended at the discretion of the investigator. In regions where enoxaparin is not available at the prefilled dose stated above, it may be substituted with another low molecular weight heparin (e.g., dalteparin [5000 IU qd], nadroparin [2850 IU qd]) or modified enoxaparin dose, per institutional standard.

Aspirin: Low-dose enteric-coated aspirin (75 mg to 100 mg PO qd) will be started 24 hours after SC-islet infusion and continued as medically indicated.

### Assessments of Islet Cell Function

Islet cell function can be evaluated at Screening and specified timepoints after infusion using a mixed meal tolerance test (MMTT). Subjects can undergo HbA1c assessments after treatment with SC-islets. Subjects can wear a continuous glucose monitoring (CGM) device from approximately 4 weeks before SC-islet infusion (baseline) and for at least 365 days after infusion with SC-islets. Exogenous insulin dose can be assessed regularly for all subjects, starting with a baseline

### Example 2: Modified Immunosuppression Regimen

SC-islets are prepared in a manner substantially the same as that described in Example 1. The peri-perfusion process is also substantially the same as that described in Example 1.

Rabbit anti-thymocyte globulin (THYMOGLOBULIN®; ATG) will be used for induction for subjects who were not previously treated with T-cell depleting therapy (TCDT; e.g., ATG or alemtuzumab). The total dose of ATG is 3 mg/kg given as an IV infusion in three separate doses over the course of a) Day −2 through Day −1 as described in Example 1, b) Day −14 through Day −13, or c) Day −14 through Day −1. The 1^{st} dose is 0.5 mg/kg infused over 6 to 12 hours, the 2nd dose is 1.0 mg/kg infused over ≥6 hours, and the 3rd dose is 1.5 mg/kg infused over ≥6 hours. The doses will be administered prior to the SC-islet infusion procedure, which will occur on Day 1. The following pre-medications are administered before/during infusion of ATG:

1. Acetaminophen/paracetamol: 500 mg to 650 mg PO/rectal (PR), approximately 30 minutes before and 500 mg to 650 mg mid-way through ATG infusion.

2. Diphenhydramine: 50 mg PO, approximately 30 minutes before and 50 mg mid-way through ATG infusion. In regions where diphenhydramine is not available, it may be substituted with another H1 antihistamine per institutional standards (e.g., clemastine, chlorphenamine).

3. Methylprednisolone: 1 mg/kg IV, approximately 1 hour before and as needed during first ATG infusion only on Day −2.

4. Pentoxifylline: 400 mg PO 3 times daily (tid) approximately 1 hour before first ATG infusion on Day −2 and continued through Day 8. However, if the subject previously received T-cell depleting therapy (TCDT; e.g., ATG or alemtuzumab), then subjects will receive Basiliximab for induction. Basiliximab will be administered 20 mg IV on Day 1 (≤2 hours before infusion) and on Day 5, diluted to 50 mL with normal saline and infused over approximately 20 to 30 minutes, per package insert.

Etanercept is administered peri-transplant 50 mg IV on Day 1 (approximately 1 hour before SC-islet infusion), then 25 mg subcutaneously on Days 4, 8, and 11. Alternatively, etanercept may be administered subcutaneously. Maintenance immunosuppression and monitoring is continued through the end of the study or until discontinuation of immunosuppression therapy.

Antimicrobial prophylaxis is administered as described below or may be superseded by local guidelines at the discretion of the clinician:

Trimethoprim/Sulfamethoxazole: Trimethoprim/sulfamethoxazole will be administered at a dose of 80 mg/400 mg PO once daily (qd) beginning on Day 2 for 6 months after SC-islet infusion. If a subject is unable to take trimethoprim/sulfamethoxazole, they will be treated on a case-by-case basis as medically indicated.

Clotrimazole: Clotrimazole will be administered as 1 troche PO 4 times per day (qid) beginning on Day −1 before the SC-islet infusion when basiliximab is used for induction (Day −2 when induction is with ATG) and continuing for 3 months after the last SC-islet dose. Alternatively, antifungal prophylaxis per standard practice at each site may be administered instead of clotrimazole.

Valganciclovir: Valganciclovir will be administered at a dose of 450 mg PO qd beginning on Day −1 before the SC-islet dose when basiliximab is used for induction (Day −2 when induction is with ATG) increasing to 900 mg qd by Day 13 and continuing for at least 6 months after SC-islet infusion. If the subject is cytomegalovirus (CMV) negative and the transplanted kidney was from a CMV negative donor, viral prophylaxis may be substituted by acyclovir at a dose of 400 mg PO bid.

Heparin: Heparin will be administered at a dose of 70 U/kg body weight of recipient, divided equally among the islet bags, given with SC-islet infusion, followed by 3 U/kg/h IV for the next 4 hours. From the 5th through the 48th hour post-transplant, heparin will be titrated to achieve and maintain activated partial thromboplastin time (aPTT) between 50 to 60 seconds. At the investigator's discretion, a comparable site-specific method and value may be used instead.

Enoxaparin Enoxaparin will be administered at a dose of 30 mg subcutaneously bid, with the first dose given 48 hours after the transplant procedure (when heparin is discontinued). Enoxaparin will be continued through Day 7 after SC-islet infusion. The dose can be modified or extended at the discretion of the investigator. In regions where enoxaparin is not available at the prefilled dose stated above, it may be substituted with another low molecular weight heparin (e.g., dalteparin [5000 IU qd], nadroparin [2850 IU qd]) or modified enoxaparin dose, per institutional standard.

Aspirin: Low-dose enteric-coated aspirin (75 mg to 100 mg PO qd) will be started 24 hours after SC-islet infusion and continued as medically indicated.

### Assessments of Islet Cell Function

Islet cell function can be evaluated at Screening and specified timepoints after infusion using a mixed meal tolerance test (MMTT). Subjects can undergo HbA1c assessments after treatment with SC-islets. Subjects can wear a continuous glucose monitoring (CGM) device from approximately 4 weeks before SC-islet infusion (baseline) and for at least 365 days after infusion with SC-islets. Exogenous insulin dose can be assessed regularly for all subjects, starting with a baseline

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure can be employed in practicing the present disclosure. It is intended that the following claims define the scope of the present disclosure and that methods and structures within the scope of these claims and their equivalents be covered thereby.

1. A method of treating a subject having diabetes, comprising administering to the subject: a) a composition comprising a population of stem cell-derived islet cells, and b) one or more immunosuppressive agents; wherein the subject previously received an organ transplant.

2. The method of claim 1, wherein the subject previously received a kidney transplant.

3. The method of claim 1, wherein the subject previously received a liver transplant.

4. The method of claim 1, wherein the composition comprises about 1×10⁸ to about 10×10⁸ cells.

5-6. (canceled)

7. The method of claim 1, wherein:
(a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, or 80-90% of the cells in the composition express C-peptide and ISL1;
(b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the composition express glucagon but not somatostatin; and/or
(c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the composition express somatostatin but not glucagon.

8-14. (canceled)

15. The method of claim 1, wherein between 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, 40-50% of the cells in the composition are NKX6.1⁺/ISL1⁺ cells.

16. The method of claim 1, wherein between 8-20%, 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, or 40-50% of the cells in the composition are NKX6.1⁻/ISL1⁺ cells.

17. (canceled)

18. The method of claim 1, wherein less than 6% or between 0-6%, 0-4%, 0-2%, 0-1%, 1-6%, 1-5%, 1-4%, 1-3%, 1-2%, 2-3%, or 3-6% of the cells in the composition are NKX6.1⁻/ISL1⁻ cells.

19. The method of claim 1, wherein:
at least 30% of the cells in the composition are NKX6.1-positive, ISL1-positive cells;
at least 15% of the cells in the composition are NKX6.1-negative, ISL1-positive cells;
less than 12% of the cells in the composition are NKX6.1-negative, ISL1-negative cells; and/or
less than 20% of the cells in the composition are NKX6.1-positive, ISL1-negative cells.

20-21. (canceled)

22. The method of claim 1, wherein a plurality of cells in the composition are in one or more cell clusters.

23. The method of claim 22, wherein the one or more cell clusters are about 50 μm to about 500 μm, about 50 μm to about 400 μm, about 50 μm to about 300 μm, about 60 μm to about 400 μm, about 60 μm to about 300 μm, about 60 μm to about 250 μm, about 75 μm to about 400 μm, about 75 μm to about 300 μm, about 75 μm to about 250 μm, about 125 μm to about 225 μm, about 130 μm to about 160 μm, about 170 μm to about 225 μm, about 140 μm to about 200 μm, about 140 μm to about 170 μm, about 160 μm to about 220 μm, about 170 μm to about 215 μm, or about 170 μm to about 200 μm in diameter.

24. (canceled)

25. The method of claim 1, wherein the one or more immunosuppressive agents are an anti-thymocyte globulin binding moiety, an anti-CD25 binding moiety, an anti-CD52 binding moiety, or a high dose of an immunosuppression maintenance agent.

26. The method of claim 1, wherein the one or more immunosuppressive agents is an anti-thymocyte globulin binding moiety.

27. The method of claim 26, wherein the anti-thymocyte globulin binding moiety is administered at a dose of less than 6.0 mg/kg.

28. The method of claim 1, wherein if the subject was previously administered an anti-thymocyte globulin binding moiety, then the one or more immunosuppressive agents is not an anti-thymocyte globulin binding moiety.

29. (canceled)

30. The method of claim 1, wherein the one or more immunosuppressive agents is basiliximab.

31. (canceled)

32. The method of claim 1, wherein the one or more immunosuppressive agents is alemtuzumab.

33. The method of claim 30, wherein the basiliximab is administered at a dose of 10-30 mg, 15-25 mg, 18-22 mg, or 19-21 mg.

34. The method of claim 1, wherein the one or more immunosuppressive agents is administered on the same day as the composition comprising a population of stem cell-derived islet cells.

35-43. (canceled)

44. The method of claim 1, wherein the method comprises administering to the subject tacrolimus (e.g., 0.001-0.05 mg/kg of tacrolimus).

45-91. (canceled)

92. The method of claim 44, wherein the administration of tacrolimus is started after the day of the administration of the composition comprising the population of cells (Day 1).

93-95. (canceled)

96. The method of claim 92, wherein the tacrolimus dose is adjusted to maintain a whole blood 12-hour trough of 3 to 6 ng/mL.

97-100. (canceled)

101. The method of claim 92, wherein 1-10 months, 1-8 months, 1-6 months, 3-10 months, 3-8 months, 3-6 months, or 5.5-6.5 months after the subject was administered the composition comprising a population of stem cell-derived islet cells and if the subject is not also being administered sirolimus, the tacrolimus dose is adjusted to maintain a whole blood 12-hour trough of 4-10 ng/ml, 5-9 ng/ml, or 6-8 ng/ml.

102. The method of claim 92, wherein 5.5-6.5 months after the subject was administered the composition comprising a population of stem cell-derived islet cells and if the subject is not also being administered sirolimus, the tacrolimus dose is adjusted to maintain a whole blood 12-hour trough of 6-8 ng/mL.

103-116. (canceled)

117. The method of claim 1, wherein the subject is administered a second composition comprising a population of cells at a later point in time than the administration of the first composition; wherein the second composition comprises a population of stem cell-derived islet cells.

118-142. (canceled)

143. The method of claim 1, wherein the composition comprises a plurality of NKX6.1-positive; ISL1-positive cells, wherein the NKX6.1-positive; ISL1-positive cells express higher levels of MAFB, CHGA, ESRRG, SIX2, HOPX, IAPP and/or UCN3 than NKX6.1-positive; ISL1-positive cells from the pancreas of a healthy control adult subject.

144. The method of claim 1, wherein the composition comprises a plurality of NKX6.1-positive; ISL1-positive cells, wherein the NKX6.1-positive; ISL1-positive cells express SIX3, MAFA, CHGB, RBP4 and/or FXYD2 at a lower level than a NKX6.1-positive, ISL1-positive cell from the pancreas of a healthy control adult subject.

145-158. (canceled)

159. The method of claim 22, wherein at least 90%, 93%, 95%, 96%, 97%, 98%, 99% or 100% of the clusters in the composition are less than 200 μm in diameter; or wherein 90-100%, 95-100%, 90-95%, 95-97%, or 97-99% of the clusters in the composition are less than 200 μm in diameter.

160-161. (canceled)

162. The method of claim 1, wherein the subject did not receive a cadaveric islet transplant prior to receiving the composition.

163. The method of claim 1, wherein the transplanted organ is not from a non-human animal.

164. (canceled)

165. The method of claim 1, wherein the subject has Type 1 Diabetes.

166-169. (canceled)

170. The method of claim 1, wherein the composition comprising the population of stem cell-derived islet cells is administered via the hepatic portal vein.

171. The method of claim 1, wherein the composition comprising the population of stem cell-derived islet cells is administered between the rectus sheath and the musculus rectus abdominis.

172. The method of claim 1, wherein the composition comprising the population of stem cell-derived islet cells is administered intra-muscularly.

173. The method of claim 1, wherein the composition comprising the population of stem cell-derived islet cells is administered into the peritoneal cavity.

174-176. (canceled)
